SEQUENCE LISTING (27 sequences)

SEQ ID NO: 1 - protein, 1081 aa, Homo sapiens
MPRRKQEQPKRLPSHVSRQEEAEGELSEGEHWYGNSSETPSEASYGEVQENYKLSLEDRIQEQSTSPDTSLGSTTPSSHTLELVALDSEVLRDSLQCQDHLSPGVSSLCDDDPGSNKPLSSNLRRLLEAGSLKLDAAATANGRVESPVNVGSNLSFSPPSHHAQQLSVLARKLAEKQEQNDQYTPSNRFIWNQGKWLPNSTTTCSLSPDSAILKLKAAANAVLQDKSLTRTEETMRFESFSSPFSSQSASSTLAALSKKVSERSLTPGQEHPPPASSFLSLASMTSSAALLKEVAARAAGSLLAEKSSLLPEDPLPPPPSEKKPEKVTPPPPPPPPPPPPPPPQSLELLLLPVPKGRVSKPSNSASEEESGKPFQCPICGLVIKRKSYWKRHMVIHTGLKSHQCPLCPFRCARKDNLKSHMKVHQHQDRGETFQCQLCPFTSSRHFSLKLHMRCHQHFLRTEAKVKEEIPDPDVKGSPHLSDSACLGQQREGGGTELVGTMMTSNTPERTSQGGAGVSPLLVKEEPKEDNGLPTSFTLNAADRPANHTKLKDPSEYVANSASALFSQDISVKMASDFLMKLSAANQKEPMNLNFKVKEEPKEGESLSTTLPRSSYVFSPESEVSAPGVSEDALKPQEGKGSVLRRDVSVKAASELLMKLSAESYKETQMVKIKEEPMEVDIQDSHVSISPSRNVGYSTLIGREKTEPLQKMPEGRVPPERNLFSQDISVKMASELLFQLSEKVSKEHNHTKENTIRTTTSPFFSEDTFRQSPFTSNSKELLPSDSVLHGRISAPETEKIVLEAGNGLPSWKFNDQLFPCDVCGKVFGRQQTLSRHLSLHTEERKYKCHLCPYAAKCRANLNQHLTVHSVKLVSTDTEDIVSAVTSEGSDGKKHPYYYSCHVCGFETELNVQFVSHMSLHVDKEQWMFSICCTACDFVTMEEAEIKTHIGTKHTGEDRKTPSESNSPSSSSLSALSDSANSKDDSDGSQKNKGGNNLLVISVMPGSQPSLNSEEKPEKGFECVFCNFVCKTKNMFERHLQIHLITRMFECDVCHKFMKTPEQLLEHKKCHTVPTGGLNSGQW

SEQ ID NO: 2 - protein, 616 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 3 - protein, 1522 aa, Homo sapiens
MSRNDKEPFFVKFLKSSDNSKCFFKALESIKEFQSEEYLQIITEEEALKIKENDRSLYICDPFSGVVFDHLKKLGCRIVGPQVVIFCMHHQRCVPRAEHPVYNMVMSDVTISCTSLEKEKREEVHKYVQMMGGRVYRDLNVSVTHLIAGEVGSKKYLVAANLKKPILLPSWIKTLWEKSQEKKITRYTDINMEDFKCPIFLGCIICVTGLCGLDRKEVQQLTVKHGGQYMGQLKMNECTHLIVQEPKGQKYECAKRWNVHCVTTQWFFDSIEKGFCQDESIYKTEPRPEAKTMPNSSTPTSQINTIDSRTLSDVSNISNINASCVSESICNSLNSKLEPTLENLENLDVSAFQAPEDLLDGCRIYLCGFSGRKLDKLRRLINSGGGVRFNQLNEDVTHVIVGDYDDELKQFWNKSAHRPHVVGAKWLLECFSKGYMLSEEPYIHANYQPVEIPVSHKPESKAALLKKKNSSFSKKDFAPSEKHEQADEDLLSQYENGSSTVVEAKTSEARPFNDSTHAEPLNDSTHISLQEENQSSVSHCVPDVSTITEEGLFSQKSFLVLGFSNENESNIANIIKENAGKIMSLLSRTVADYAVVPLLGCEVEATVGEVVTNTWLVTCIDYQTLFDPKSNPLFTPVPVMTGMTPLEDCVISFSQCAGAEKESLTFLANLLGASVQEYFVRKSNAKKGMFASTHLILKERGGSKYEAAKKWNLPAVTIAWLLETARTGKRADESHFLIENSTKEERSLETEITNGINLNSDTAEHPGTRLQTHRKTVVTPLDMNRFQSKAFRAVVSQHARQVAASPAVGQPLQKEPSLHLDTPSKFLSKDKLFKPSFDVKDALAALETPGRPSQQKRKPSTPLSEVIVKNLQLALANSSRNAVALSASPQLKEAQSEKEEAPKPLHKVVVCVSKKLSKKQSELNGIAASLGADYRWSFDETVTHFIYQGRPNDTNREYKSVKERGVHIVSEHWLLDCAQECKHLPESLYPHTYNPKMSLDISAVQDGRLCNSRLLSAVSSTKDDEPDPLILEENDVDNMATNNKESAPSNGSGKNDSKGVLTQTLEMRENFQKQLQEIMSATSIVKPQGQRTSLSRSGCNSASSTPDSTRSARSGRSRVLEALRQSRQTVPDVNTEPSQNEQIIWDDPTAREERARLASNLQWPSCPTQYSELQVDIQNLEDSPFQKPLHDSEIAKQAVCDPGNIRVTEAPKHPISEELETPIKDSHLIPTPQAPSIAFPLANPPVAPHPREKIITIEETHEELKKQYIFQLSSLNPQERIDYCHLIEKLGGLVIEKQCFDPTCTHIVVGHPLRNEKYLASVAAGKWVLHRSYLEACRTAGHFVQEEDYEWGSSSILDVLTGINVQQRRLALAAMRWRKKIQQRQESGIVEGAFSGWKVILHVDQSREAGFKRLLQSGGAKVLPGHSVPLFKEATHLFSDLNKLKPDDSGVNIAEAAAQNVYCLRTEYIADYLMQESPPHVENYCLPEAISFIQNNKELGTGLSQKRKAPTEKNKIKRPRVH

SEQ ID NO: 4 - protein, 82 aa, Artificial Sequence
  ZnF1-3 binding domain
FQCPICGLVIKRKSYWKRHMVIHTGLKSHQCPLCPFRCARKDNLKSHMKVHQHQDRGETFQCQLCPFTSSRHFSLKLHMRCH

SEQ ID NO: 5 - protein, 253 aa, Artificial Sequence
  ZnF4-9 binding domain
FPCDVCGKVFGRQQTLSRHLSLHTEERKYKCHLCPYAAKCRANLNQHLTVHSVKLVSTDTEDIVSAVTSEGSDGKKHPYYYSCHVCGFETELNVQFVSHMSLHVDKEQWMFSICCTACDFVTMEEAEIKTHIGTKHTGEDRKTPSESNSPSSSSLSALSDSANSKDDSDGSQKNKGGNNLLVISVMPGSQPSLNSEEKPEKGFECVFCNFVCKTKNMFERHLQIHLITRMFECDVCHKFMKTPEQLLEHKKCH

SEQ ID NO: 6 - protein, 271 aa, Artificial Sequence
  TOPBP1 binding domain
NHTKLKDPSEYVANSASALFSQDISVKMASDFLMKLSAANQKEPMNLNFKVKEEPKEGESLSTTLPRSSYVFSPESEVSAPGVSEDALKPQEGKGSVLRRDVSVKAASELLMKLSAESYKETQMVKIKEEPMEVDIQDSHVSISPSRNVGYSTLIGREKTEPLQKMPEGRVPPERNLFSQDISVKMASELLFQLSEKVSKEHNHTKENTIRTTTSPFFSEDTFRQSPFTSNSKELLPSDSVLHGRISAPETEKIVLEAGNGLPSWKFNDQL

SEQ ID NO: 7 - DNA, 7660 bp, Homo sapiens
gacattagtgttaggggctcggagacacagagcgcggccatagacaccgccgcaccggcactcatttatttatacctcccatcacacacgcgagcataggacacacacacactcacacctattagatccgtttccattgaagaatattacgctcggggacaagccaggtgcacgaccaaaaatttaaaaaaaaaaaaaaaaggaaaaaagaaaggaaagaaaagaagaaaacccctcttctggctccaggaaacaagcttcaacccattgcacacaccggagaaggggtttccccctgcccgcccgcccgccccccaactacctccccgctcctgccagtgtctgcctctctgcccccacgggggtttatttgatctcacattaaccaaggaggagaatgtgagaaaagggggaaaatgcccaggaggaagcaggagcagcccaagcgccttccctcacatgttagtaggcaggaagaggcggagggagagctcagtgaaggagaacactggtatggaaactcttcagagactccgtcagaagcatcctatggggaagtccaggagaactataagttgtctctggaggaccggatccaggagcagtccacgtccccggacacctccttgggaagcacgactcccagcagccacacgttggagctggtggcactggacagtgaggtcctgcgagactcactgcagtgtcaagatcacctttccccgggagtgtcttctttgtgtgacgatgacccaggctccaacaagcccctgagcagcaatttgaggcggctgctggaggctggttccctcaaactcgatgctgcagccacggctaatggcagagtggagtcccccgtaaacgttggctcgaacctctccttttccccgccttcccaccacgcccagcagctcagtgttctggccaggaagttggccgagaagcaggaacagaatgaccaatacactccaagtaaccgttttatatggaatcaaggtaagtggttgccaaactcaaccaccacctgcagcttgtctccggattcagccatcctaaaactgaaagctgcagccaatgccgttctgcaggacaaatctctcaccaggactgaggagaccatgcgatttgagtccttttcctccccttttagctcccagtctgccagttctaccttggccgctttgtccaagaaggtcagtgaaagaagtttgactcctggtcaggagcaccctccaccggccagctccttcctttccctggcttctatgacctcctcagcggcccttctgaaggaggtggccgcaagggctgcgggcagtcttctggctgagaaatcatcgctgctgcctgaggaccctctaccgcccccgccttcagagaagaaaccagaaaaagtcactccgccacctccaccgccacctccaccacctccaccaccaccaccacaatccctggaattattattactcccagttcctaagggaagagtttctaaaccctccaattcagcctcagaagaggaaagtggaaagcctttccagtgtccaatatgtggtttggttattaaaaggaagagttactggaagcggcacatggtgattcacacaggtttaaaaagtcatcagtgtccgctctgtccattccggtgtgctcgcaaggacaatctcaaatcccacatgaaggttcatcagcaccaggatcggggagagacctttcagtgccagctgtgcccttttacttcctcacgccacttcagcctgaaactccacatgcgttgccatcagcacttcctgaggacagaagccaaggtgaaggaggagatcccagacccagatgtcaagggatctccccacctcagtgacagtgcctgcctggggcagcaaagggaaggaggagggacagagctagtggggaccatgatgacgtctaacactccagagaggactagccagggaggggctggcgtctcgcctttgctggtgaaggaggaacccaaggaagataacggcctgcccacctcctttactttgaatgctgccgacaggcccgccaaccacacaaagctgaaagacccctccgagtatgtggccaacagtgcgtcagcattgttcagccaggacatctctgttaagatggcgtctgattttctcatgaagctgtcagctgcaaatcagaaggagcccatgaatcttaattttaaagtgaaagaggagcctaaggaaggggagtccctaagcacgactttgcctcggtccagctatgtgttcagcccggaatctgaagtgtcagccccaggcgtctctgaggacgcactaaagccccaggaagggaagggaagtgtgctaaggcgggatgtgtcagtcaaagcagcctctgaacttctcatgaaactctcagcggaaagctacaaggaaacacagatggtgaagattaaagaggaacccatggaggttgacatccaggactcccatgtctcgatatcacccagccggaatgttggctacagcactttaatcgggcgagagaaaaccgaacccttacagaagatgccagagggcagagtacccccagagagaaacctcttcagtcaggatatctctgtgaaaatggcttccgagctcctctttcaactgtcagaaaaagtgagcaaagagcacaatcatacaaaagaaaacaccatccggaccacgaccagccctttcttttcagaagacacatttagacaatcaccattcacctccaattcaaaagaactgctgcccagtgactccgtgctgcacggaagaatatcagctccagaaacagaaaagatagtcctagaggcaggaaatggattaccatcctggaaattcaatgaccagctttttccctgtgacgtgtgtgggaaagtgtttggccgacagcagacattgtcccgacacctctcgctgcacacagaggaaagaaaatacaaatgccacttgtgcccctatgctgctaagtgccgtgcaaatctgaaccagcacttgaccgtccattccgtgaagctggtgagtacagacaccgaggacattgtcagcgccgtcacctctgaaggcagtgatgggaagaaacatccttattattacagttgtcacgtgtgtggatttgagaccgagctcaatgtccagtttgtcagccacatgtcactccacgtggacaaggagcagtggatgttttcgatctgctgcactgcctgcgacttcgtcaccatggaggaagcagagataaagactcacattggcaccaagcacacaggggaagacaggaagacccccagcgaatcaaatagcccctcttcatcctccctctcagctctgagtgattcagccaacagcaaagatgattcagatggctcccagaaaaacaagggcgggaacaatctgctggtcatctctgtcatgcctgggagccagccctcactgaacagtgaggaaaagccagagaaagggttcgaatgtgttttttgcaactttgtctgcaagacgaagaacatgtttgagcgtcatctgcagatacacctcatcacccggatgtttgagtgtgatgtgtgccacaagttcatgaagacccccgaacagctgctggagcataagaaatgccacactgtccccaccggtgggctcaagtaaggaaactcaggacagtggtgagtttcagactcctctaggtgcccattctgcatttattccaccaaccgccccgctgccatggagtgccacctcaagacccactacaagatggagtacaagtgccggatctgccagacggtgaaggccaaccagctggagctggagacgcacacccgggagcaccgcctgggcaaccactacaagtgcgaccagtgcggctacctgtccaagaccgccaacaagctcatcgagcacgtgcgcgtccacaccggggagcggcccttccactgtgaccagtgcagctacagctgcaagcgcaaggacaatctcaacctgcacaagaagctgaagcacgccccacgccagaccttcagctgcgaagagtgcctgttcaagaccacacaccctttcgtcttcagccgccacgtcaagaagcaccagagtggggactgccccgaggaggacaagaagggcctgtgtccagcccccaaggaaccggccggcccgggggccccgctcctggtggtcgggagctcccggaatctcctgtctcccctgtcagttatgtctgcctcccaggctctgcagaccgtggccctgtcggcagcccacggcagcagctcagagcccaacctggcactcaaggctttggccttcaacggctcccctttgcgctttgacaagtaccggaactcagattttgcccatctcattcccttgacaatgttataccccaagaaccacttggatctcacattccaccctccccgacctcagactgcgcctcccagcatcccctcacccaaacactccttcctggcctatctcggactgagagaaagagcagagactgtctgagggcagccatgttctgtaccaaaaacagagagacaaaagacaaaaaaaaaaaaaaaaaccacaaaacttaaacacaaccccagcaggtgtatgttgctgcaaaacctacagaccccgatgggtctggaacatgtgtactgtatatctttagtaaggaatagaaaattggctctgtgtgtatacctattgcattgacctgaaagctgctttatccaatcttcagagaggtgacctactgcatacttctaccttcagaggcatgcctccccagccacccactcccactctcagcccttctccgtacttttctctgaaaggaatcttgtcttgttaaaccctaaagagagtgtccttaatagcaatcagcacttgtaagcttatatactggtgcatttggttttctgttgggtgaatgcggtgtgtgggcgtttgtggattctgaaagagaaagccgtgtgtcgtgtgccatgacatttctattgcacattctttgtactggcttctttaacagcgatgaacgttcttttccctcctgggttgttcatccacgacagtctctccctgtgctccttcatcacctttccctctctctttgatggctacagagtggtagggcctggtgcttagtcgatgaaggaatggtagccatctacagtgctgctggagatttccgccagagcgctggagaccttgcgctcagatcttctctggtgatgaagaggcgaggaatcaagtgactgatctggaagaaatatctcgcagcactgcagctaacatcacagaacttaagtgtgttttgtgtgtgtgcccacacgtagacaaatgtgtacggtgcacacacacagtgcatcattttttaagggcagattatatatatatatgagatgtattaattcagtggttaccatttgtttgcaggaaaaagaaatgtatgggtgaaaaaatttatggttgataaatccagccaaggagattaaaaggggtttggataaattctgggtataaatgctcagactaaaaaaaagaaatggcagttttgcacagtgctatggtcttgcactagtttttgtttctcatctgaaaaaaaaaaagtaaaataaaaggaagaaaatgtaccttttttatggaatgagtagactgtatgtttgaagatttagccacaacctctttgacatataatgacgcaacaaaaaggtgctgtttagtcctatggttcagtttatgcccctgacaagtttccattgtgttttgccgatcttctggctaatcgtggtatcctccatgttattagtaattctgtattccattttgttaacgcctggtagatgtaacctgctaggaggctaactttatacttatttaaaagctcttattttgtggtcattaaaatggcaatttatgtgcagcactttattgcagcaggaagcaggtgtgggttggttgtaaagctctttgctaatcttaaaaagtaatgggtgatttaaaaagaaaaaaggaaaaaaatctttggctgaatatgttcattgcttgtatttttaaaacaacagaatttccagtatgaaacaggctgaaagagcaggaagaaatgttctttgtataataatgggaagtttggaatataaaagtttatatattatttatctattggagaactggtgtacaggaggaacattttcttactgtgttgctgttttccatcatgtgttatcctaagagttggggttttttaaaatctgtttcaccaggggaaaataaaagcatccctaatgttcttcctctagtcagtttgctttgctgaaagctccctggaaattagtacagttctacaggaagccagaagactgtactgtctgtccacaccatggtctcctcagtcccctccacgccagcccctcttccccacacctagtaggtgtgctacacctccctttgccagtcatcccgccatggatggggagcctacttggtgccctggcaaacctgtttacactaaggagatgcccacagggagcgggaagctcccaaaccctcagtcattctccagcagagacctttgctcttggccaggtttgaaacgatgttcaacctcgccacgtcaatgtctcagtaaaagacactatagatagttatggacaatacactccaagaatactgggcttatgggtgggttggttttttgttttgttttgttttgttttcaagtcatgtctttatccccatataatggagaatgtgccacttggctaataatgtgttttcaaaactacaagttcagtgatgacaggaaagagggctaaagtagtcccacagaggctggtagaaaaggcgaatcacacttaagcaccagcaggttaaaaacctgaaagggtggccctgacaattcagactataggttttacagcatgccacttgtgcaataggaggtctagggttgaaactttcaagaacgagaggaggtgaaggaaggagacgtttactgctcaaatgccatgcatttcactgcttgccactgaacttgtatttgaatgacttagtaatgcttaatataattgggcaacttttttagcactttggaaagagtcatcaatctttctggtaaattataaaagttttctgagtgagaaaaggtgtgtttggagtttgtttgactttttatattattattgttattaataaagaaaaaaatctacagcatttttcagactccaccctagaaaatatgtacccctgaatttggtttgcagtttgatactcagggaaggatgtattccatagatatcctttccgtataaaacactaacatctttgagaaattcacctaccaactaacttcaattgttgtcactatgcattcttcaatgaaaactagcttatttttccatatagtaatgcagttagggttctcagcactttctttcttctatcctttttttaactcttcatattatgttcagatgatcatactgtcaaggtttgtgtacattactgaaaatttgtattgtataaagctttttgcattacgaggctgtacagggtcattttgacagtaactggtatattcctttgcatcttatgttgcattgccaatttctagtgtatccagtttgaaagtataatatactctaattaaaaaaaaaaaaaggttggctata

SEQ ID NO: 8 - DNA, 4847 bp, Homo sapiens
ctcgggccaataactgcgcagcgcgcgggacccgggtggggaagctggagctgttgcggggtccgcggggaagtcttggcggtggagccatggtcggccaactgagcgagggggccattgcggccatcatgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaagccggactactttagttctgtggccacagtggtgtatcttcgcaaagagaactgcatgtaccaagcctgcccgactcaggactgcaataagaaagtgattgatcaacagaatggattgtaccgctgtgagaagtgcgacaccgaatttcccaatttcaagtaccgcatgatcctgtcagtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatgtgagaggagcagtgccaatcgggcagaagtttgcaaataggcagaatggaatcgatttcctcccacctccgtgtgacgatcccatgttagctacacagtgcagaggctcttgatggtggactaagcaatttcccccctcgtgcgcatctcagaacccatcggtaggcaaaggaaaatacgctcaggtggttgtggtgtagactgtgtcaggcctacggagtcagccagtggctagcgcaagaccagtcactccctctgccttcaggcttctgtcaatttcattatcatcaagcaggaattatgtcgtaagtcactgaccctaactgcagaccatgaagtaaattatgtaactaggtttttgcttctccagtggtgaccacccccccccatccccgctcacaacttgggttcttctcagcggggcgagctgagaagcggtcatgagcacctggggattttagtaagtgtgtcttcctagaattcgaaggctctctctttctagaggtgctacatagttggtaatgcttggaatggcaatagggtagaatgattaatcaaaggcatatcttctatatctgaagagtatccttccttcagggtttaatagactgagtcagatgggtctgatattaatcaaaattgtctcttctgaggaccgctgataagcattgacttgccgtcccctaaggaaatccgagcggctacaaagcgtttctttacttctcacttcaattaatgctgcgcttcgcttggtgagtgcgtactttttctacctgtacacattcctgcattcatgtattttgttttttttgactaaagctatgttacatggaaaggattttgaagccttttgtttcccttgctttgttttaataaacagtatattctttggttgtgaatcctactttctttgaatgcaaagagttcctataactggaaagcaattaattagccttcataataaatgttcacttgggggggatgttaactcattataaacccgaagattagtccaaggcatggagatccttcttcctagtgtttgcagccctgaaatgcatctttcaaagcatgaaaacactaaaaacaaaaagccattttgcctgaggatgctgatgatctggcacttgggattttattgatgtttacgcagcagtctaacaccaaccacgctttgaaatgtgtacagacagtgagctggtaagaaaacagtaattatgctagtgggcctttcagtcagcaaaagcatgctcgctctgtgtgttcctaatcatattaattatctatccggtggctgcaacacaccgcctgccattggccgcacatctcgccgtcgtaccccggcagtgcggcggtcactctgcagccagaggacctgctgttcatcactgcacatgccgcctgcggaggcttttggatatggggagtgatggtgtcctgtctgtctcccccctcggtgtctgccgttgacataggggccagccagccctagacgggatgacttccgttcctgaggacagacacagagggactcctgctcagcctcactaattgtttagacacattccttcctacccttctctagtctcaggagatggtaactgggtcgcatttcagtctctgactgaggcctcagccatttttacggaagttcttttctgtctgaccttgcttataaagcatcgacgagaaaattacagtcttcaaccctcttctggattgacaaattgtggctgggaagtggcgatctagctttcagcccagtaaccagtctttcatgcctactactcccagcattccctcctctccccacgtgtctgtccacacagtgaagaggcctgaccagccgtggtaccaggacaggacgtgtccagggaacgctgacacctgtcctcgcgccttctcagtggccagcgtgatgaaaccagcacgtctccgtggatgtgattgggaacccaggggcagtgccagggggagggctccctcgaggaggctgtttctaacagatttccccactcaaagatcagatcaccagcagaggagcatcagaaactggctccactgttgggcttttcagagattttggtccctgcgggttgcctaaatagattctggcccacagtttacctcgaaaggctgttgatgttgttctgtttctcctctttcacttagagatcaatgttgattttgcgtacaccatgacatcagcgttaggcaattaggagaaaaaaatctaatcatttcgcctttatttcaagtggttcttgaattccctccagtctcattgtgaaaggggcagggaaaaaaataaaagggtaataatctgatttctgtccatatttccagtgttttatgctttccattaaaaccttgctaatctctcgcgtgctgttccttttttgaactgcagggtcaaaaatgttcccctcccccacttctgtttcctgagtgggctgtcttcaaaaagtataaatactattgatcgtggtatttaatacactaatgaaacccatggttattgactacataattaatatactgtccaaaagtgaagtttggccgcccagtatgtaaaggtttatattgactcatgtttctttggcaatatgccgcccaaggttttatttatagaggatgaaacaggttgtttggtctccttttcttcttttcacctgttagaaggccactattcccctagtaaactgattgtttccttgtctcaaagggaagattccccccactccccggccaccttttatgtggtttgttaactcttagtcttatatccactccgctggtagagttctggcttttataaacacctttggaagtcatcggttcctcaaaaagagagcatagatgtttaattttcacttattcaaaaaaactggtatttttgttattaagaca

SEQ ID NO: 9 - DNA, 1584 bp, Homo sapiens
attcgcgggaaggcgtttgtggtgccagagaaaagtagccagagcggcgcagtggcggccgcgttctgtggttttccgctattcccccagacccgcaccttctcggcctctttgcggagaatcgtgaccaagatgtggaacagtggattcgaaagctatggcagctcctcatacgggggagccggcggctacacgcagtccccggggggctttggatcgcccgcaccttctcaagccgaaaagaaatcaagagcccgagcccagcacattgtgccctgtactatatctcagctgctttctgccactttggttgatgaagtgttcagaattgggaatgttgagatttcacaggtcactattgtggggatcatcagacatgcagagaaggctccaaccaacattgtttacaaaatagatgacatgacagctgcacccatggacgttcgccagtgggttgacacagatgacaccagcagtgaaaacactgtggttcctccagaaacatatgtgaaagtggcaggccacctgagatcttttcagaacaaaaagagcctggtagcctttaagatcatgcccctggaggatatgaatgagttcaccacacatattctggaagtgatcaatgcacacatggtactaagcaaagccaacagccagccctcagcagggagagcacctatcagcaatccaggaatgagtgaagcagggaactttggtgggaatagcttcatgccagcaaatggcctcactgtggcccaaaaccaggtgttgaatttgattaaggcttgtccaagacctgaagggttgaactttcaggatctcaagaaccagctgaaacacatgtctgtatcctcaatcaagcaagctgtggattttctgagcaatgaggggcacatctattctactgtggatgatgaccattttaaatccacagatgcagaataactggatctaactgggtacctgagatattttacagctggacctagtttcacaatctgttgtctccagctctgcatatgtctggccagggggcttctaggaagtaggtttcatctatcaaatgtctcctctgacttccttttgaaacttactgctcttctgttttattttgttttgtttgaagctcagagggagatgggcaattgacagggatgcaatccagggtgggatttcttgaggaagttacaaataagcttgttacaacatcaagatagatggaattggaaggatgctaccaggagagtacttacatagtgctcaggagtttctcttcttaaaatgtttactgctgaaagatgagcaggaccagggcgttataggcagagccctagccgagaaacctgctggcctctgcctgttttcatttcccactttggttgtgtggcattactttcagaattgcactttcctgcttgtcatgactttttgacacacttgccatgacgtgtgtttctgtgaacatgaagttctgcggtagtgcctccaggggcagaggaaaagaagaagtgttactgcattttgtacaaaataaatacagtcatatgtttaataaaacagttctattgtagtaacttgtaaaaa

SEQ ID NO: 10 - DNA, 2020 bp, Homo sapiens
aagctactcagataagaggctccaagaggacatttttggatgtgaaaaacaatgagaaggaggacaacacacatttacaatcgtcttaattttgtactcagaaaaaggatgtgaagacaatgcacagggaatacaatagtttcagatctgtgtacagtttccttttgcttcatctcctgcaacaatgtaatgaagacaccatgatatcattaacatttcacacaaaaggaaaatgaggctgaaatggtgtgggcaaggcccaggaatctggagcatccctaaccaagcaggagagcacctgggatagagaaagtgctcaagaatgttcacttactgattactacaatcaaaaaaagatacgacactaatttaccacattcttcttacttattttatgagatactattcttccaaggtggagaaagtggagaaagtagagtgacgcagctaagggagtaaatcgaccctcagccaacaagtggcaaaagcctgaagaaagtgatcaagatcactgatgaccccgctgcccatctccaagggggcgggtatcacaaccccgacgccacaccacgtatcattccgcaaaactcccgcgcctcccacgcagaactggcaagagggaaggcgagacagcagtgaacagctggtacgcagcacccacagcaccgcggcagcagctagtgccgactcccgcctagctcttttgactctgttcgcgggaagaatggggaaacagtaaggttgcggcgcctcccgcgagacgaggtacctgaggctggccccgcagtcccccgccgcaccagcaccggagcttcacaccccacttccggggtcaagtcaccgccgggaatcctgtgatcgcagaaaggtagtctcaggttccgcccctatccaagtcccgcctccactgcctctcgccctgtatctgtcaacttccgggacgccgcgcgtcactaagcagccaatctccacttccggactcatccagccccttctccacccctttcagagacagcgcgattgcgatttaggtttccgcgcatttaattggcgaagctggagcgctagtcttcgctgattggtgccgagaaatctgccccatagacacccgcggggcgcacagtttcagtcgtccgtgggtttcccgccagccgcagtcttggaccataatcatggtggacatgatggacttgcccaggtcgcgcatcaacgccggcatgctagctcaattcatcgacaagcctgtctgcttcgtagggaggctggaaaagattcatcccaccggaaaaatgtttattctttcagatggagaaggaaaaaatggaaccatcgagttgatggaaccccttgatgaagaaatctctggaattgtggaagtggttggaagagtaaccgccaaggccaccatcttgtgtacatcttatgtccagtttaaagaagatagccatccttttgatcttggactttacaatgaagctgtgaaaattatccatgacttccctcagttttatcctttagggattgtgcaacatgattgatcttgatggattttcatacgattgtaaatgagctatattaaagtctattaaaggaagcccttcttgtttgagggagagatttctgtgctttctcatatttaatttgctgtttttaagatattccaacctagagtttttgatggaactgatatattgacagttctcaccgaagtccttttataaagaattgctactccaatatatggtcagattagatgcaagaataaagcagttgtccgagtctaagtttctattttattaataaaaactaaaatggtacgtactatcggtcatttcattttcattcttttaatcatgtattcaagcacaaacttgaaatttcatagccataaggtcaagatttagacctaccaaataaaaccttgggccagctgtgttaaggatttgctcaccttttcccaaactataccttgataattatttccttgataccctacttacaaaatgaagtagatgacatt

SEQ ID NO: 11 - DNA, 5761 bp, Homo sapiens
agtcggttcccgttctgggcaatttccgggttctccagcgtttgctttggggtaggggcggcgccgagtcgggggagggggctgtgcgccgggctggcgcccgaccccagccaccgccctgcggccagcgcgtcccccgactcgccgcccggagaccccgaggctccaacgagttcagaaatgtccagaaatgacaaagaaccgttttttgtgaagtttttaaagtcttcagacaattccaaatgtttttttaaagctctcgagtccataaaagaattccaatcagaagaatatcttcagattattacagaagaagaggcattgaagataaaggagaatgatagatcactttatatctgtgacccttttagtggcgttgtctttgatcacctcaaaaagcttggctgcagaattgttggtcctcaagtagtcatattttgtatgcaccaccagcgatgtgtcccaagagccgaacatccagtttataatatggttatgtctgatgtaaccatatcttgtacaagtctggaaaaagaaaaaagggaagaagttcataaatatgtacaaatgatgggcggacgagtatacagagaccttaatgtatcagtaactcaccttattgcaggagaagttggtagcaaaaaatatttagttgctgcaaacctgaagaaacctattttgcttccctcttggataaaaacactttgggagaagtcacaagagaaaaaaataactagatatactgatataaacatggaagatttcaagtgtcctatttttcttggttgcataatctgtgtgactggcttatgtggcttagacaggaaagaagttcagcaactcacagttaagcatggaggtcaatacatgggacaattgaaaatgaatgaatgtacacacctcattgtgcaagaaccaaaaggtcagaagtatgagtgtgccaagagatggaatgtacactgtgtgaccacacagtggttttttgacagtattgagaaaggtttttgtcaggatgaatccatatacaagacagaacctagaccagaagcaaagactatgcccaattcttcaactcctaccagccagatcaacacaattgatagtcgtactctttcagatgtcagcaatatttccaacataaatgcaagttgcgtaagtgaatcaatatgtaattcacttaacagcaaactggagcctacacttgaaaatctagaaaatctggatgtcagtgcatttcaagcacctgaagatttattagatggttgtcggatatatctttgcggttttagtggcagaaagctagataaactgagaagacttattaacagtggaggtggagttcgttttaaccagctaaatgaagatgtaactcatgttattgtgggagattatgatgatgaattgaagcagttttggaataaatcagcccacaggcctcatgtagtgggagcaaagtggttgctagagtgtttcagtaaaggttatatgctttctgaagaaccatatatccatgctaattaccagccagtggaaattccagtttcacataagcctgaaagtaaagcagctcttttaaaaaagaagaacagcagcttctctaagaaagactttgctcctagtgaaaagcatgagcaagctgatgaagatctgctctctcaatatgaaaatggtagctccacagtagttgaggctaagacgtctgaagccaggccctttaatgattctactcatgctgagcccttgaatgattctactcacatttctttgcaagaagaaaaccagtcttctgtcagtcattgtgtccctgatgtttctacaattactgaagaaggcttatttagccaaaagagtttccttgttttgggttttagtaatgaaaatgaatctaacatcgcaaacatcataaaagaaaatgctgggaaaatcatgtcccttctgagcagaactgttgcggattatgctgtggttcctctgctggggtgtgaagtggaagccactgtgggagaagttgttacaaatacatggctggttacttgcatagactatcagactttgtttgatccaaagtcgaatcctctcttcacaccagttccagtaatgacaggaatgactcctttagaggattgtgttatttcatttagccagtgtgctggagcagaaaaagagtctttaacattcctagcaaacctccttggagcaagtgttcaagaatactttgttcgcaaatccaatgcaaagaaaggcatgtttgccagtactcatcttatactgaaagaacgtggtggctctaaatatgaagctgcaaagaagtggaatttacctgccgttactatagcttggctgttggagactgctagaacgggaaagagagcagacgaaagccattttctgattgaaaattcaactaaagaagaacgaagtttggaaacagaaataacaaatggaatcaatctaaattcagatactgcagagcatcctggcacacgcctgcaaactcacagaaaaaccgtcgttacacctttagatatgaaccgctttcagagtaaagctttccgtgctgtggtctcacaacatgccagacaggtcgcagcctccccagcagtaggacaaccacttcagaaggagccctcgttacacctggatacaccatcaaaattcctgtccaaggacaaactcttcaagccttcctttgatgtgaaggatgcacttgcagccttggaaactccaggacgtcccagccaacagaaaaggaaaccgagtacgccactctcagaagttattgtcaaaaacttgcaacttgctttggcaaatagctctcgaaatgctgtcgctctttctgccagccctcaactgaaagaggcccagtcagagaaggaagaagccccaaagccacttcacaaagtagtggtatgtgttagtaaaaaactcagtaagaagcagagtgaactaaatgggatcgcagcctctctaggagcagattacaggtggagttttgatgaaacagtgactcatttcatctatcaagggcggccaaatgacactaatcgggagtataaatctgtaaaagaaagaggagtacacattgtttccgagcactggcttttagattgtgcccaagagtgtaaacatcttcctgaatctctttatccacatacttataatcccaaaatgagcttggatatcagcgcagtgcaagatggccggctctgtaatagtcgactactctcagctgtgtcttcaacaaaggatgatgagccagatcctttgattttagaagaaaatgatgtagacaatatggccaccaataataaagagtcagcaccatcaaatggaagtggaaagaatgactctaaaggagttctgacacagaccttagagatgagagagaactttcagaagcagttacaggagataatgtctgcaacatcaatagtgaaaccccaagggcagaggacttccctttcaagaagtggttgtaacagcgcatcttcaacccctgacagcactcgctctgctcgcagtggacgaagtagagtcctagaggcactgaggcagtctcgtcagacagtacctgatgtcaacacagagccttcccaaaatgaacagatcatttgggatgaccctacagcaagggaggagagagcaaggcttgccagcaatttgcagtggcctagttgtcccacacaatactctgagcttcaggttgacattcaaaacttggaggattctccttttcaaaagcctttacatgattcagaaattgctaaacaggctgtctgtgatcctggaaacatacgtgtgactgaagctcccaaacacccaatctctgaagaactggaaactcccataaaagacagccacctgatccctacgcctcaagcccccagtattgcctttccactcgccaacccccctgtggctccgcaccctagagaaaagattataacgatagaggagactcatgaagaattaaaaaaacagtacatatttcagttatcatctctgaatcctcaagaacgtattgactattgtcatctgattgagaaactaggtggattggtgatagaaaagcagtgctttgatcccacctgtacacacattgttgtgggacatccacttcgaaacgagaagtatttagcctcagtggcagctgggaagtgggtgcttcatcgctcctaccttgaagcctgcaggactgctggacacttcgtgcaggaagaagactatgaatggggaagtagttccatacttgatgttctgactggaatcaatgtacagcaacgaagactagcacttgcagcaatgagatggagaaaaaaaatccagcaaagacaagaatctggcattgttgagggagcatttagtgggtggaaggttattttacatgtggatcagtctcgagaagcaggcttcaaacgccttcttcagtcaggaggagcaaaggtgctacctggtcattctgtacctttatttaaagaggccacacatcttttttctgacttgaataaactgaaaccagatgactcaggagttaatatagcagaagctgctgcccagaacgtgtactgcttgagaacagaatacattgctgattatctcatgcaggaatcacctcctcatgtagaaaattactgtctaccagaagctatttcatttattcagaataataaggaacttgggactggattatcacaaaagaggaaagctcctacagaaaaaaataaaatcaaacgacctagagtacactaatcgcatctaccctttagttaccaaacattaaatgtttttaaaaattgaaagcctgaatgtgactgtgatagatttgggtagtaatttaaagatgagtacctgaagaattctgcttcagagtataatgatgacccttcttgagttttgaacacctgaaattgtaatcactgaaatattaactgtttcttaataaaaagttacctgaaataacaacaaaatacaactcctcagctagcttgctgttaaaccacattgaagtctgttaaaagatatttatttttcttgtaaatatctgaagctgtagcttagtggaaattttagcaaggtaatggattttgctttaaaatgtctgccttacaaattcataacaacaagatttgtcagtcagcatttattcatgttttccctgatttttatcttctcaccattttacctcttttaacaggagcctgagcacaaggtttaatgaggaagctggggctataaatatgtgtgtatatatgtatatgtatgtttgtacaaatctccatgatgtttgccaagtttgaatgcgcaaaacttggaaaatgtgacaataaagaataaaagtagtaactcaaattagtattaagatgtgtttacatagataaattttttaaaagagcaccctggcatctgttcttctgttgaagcaacaaaacatttatgtaacgtaaaaaatgaattgcaggggcctgggtgcggtggctcgtgcctgtaatcccagcactttgggaggctgaggcaggcggatcgtctgaggtcgggagtttgagaccagcctgaccaacatggagaaacctcatctgtactaaaaatacaaaaaattagcccagtgtggtggcacatgcctgtaatcccagctactcaggaggctgaggcaggagaatcgcttgaacccgggaggcggaggttgcggtgagctgagatcgcgccattgcactccagcctgggcaacaagagcgaaactccatctcaaaaaaaaaaaaaaaaa

SEQ ID NO: 12 - RNA, 25 nt, Artificial Sequence
  siZNF827 siRNA 1
gggcagucuucuggcugagaaauca

SEQ ID NO: 13 - RNA, 25 nt, Artificial Sequence
  ZNF827 siRNA 2
ugauuucucagccagaagacugccc

SEQ ID NO: 14 - protein, 10 aa, Artificial Sequence
  ZNF827 RRK domain
MPRRKQPQEK

SEQ ID NO: 15 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 16 - DNA, 3090 bp, Artificial Sequence
  ZnF1-3 domain mutant DNA
gcgatcgccatgcccaggaggaagcaggagcagcccaagcgccttccctcacatgttagtaggcaggaagaggcggagggagagctcagtgaaggagaacactggtatggaaactcttcagagactccgtcagaagcatcctatggggaagtccaggagaactataagttgtctctggaggaccggatccaggagcagtccacgtccccggacacctccttgggaagcacgactcccagcagccacacgttggagctggtggcactggacagtgaggtcctgcgagactcactgcagtgtcaagatcacctttccccgggagtgtcttctttgtgtgacgatgacccaggctccaacaagcccctgagcagcaatttgaggcggctgctggaggctggttccctcaaactcgatgctgcagccacggctaatggcagagtggagtcccccgtaaacgttggctcgaacctctccttttccccgccttcccaccacgcccagcagctcagtgttctggccaggaagttggccgagaagcaggaacagaatgaccaatacactccaagtaaccgttttatatggaatcaaggtaagtggttgccaaactcaaccaccacctgcagcttgtctccggattcagccatcctaaaactgaaagctgcagccaatgccgttctgcaggacaaatctctcaccaggactgaggagaccatgcgatttgagtccttttcctccccttttagctcccagtctgccagttctaccttggccgctttgtccaagaaggtcagtgaaagaagtttgactcctggtcaggagcaccctccaccggccagctccttcctttccctggcttctatgacctcctcagcggcccttctgaaggaggtggccgcaagggctgcgggcagtcttctggctgagaaatcatcgctgctgcctgaggaccctctaccgcccccgccttcagagaagaaaccagaaaaagtcactccgccacctccaccgccacctccaccacctccaccaccaccaccacaatccctggaattattattactcccagttcctaagggaagagtttctaaaccctccaattcagcctcagaagaggaaagtggaaagcctcagcacttcctgaggacagaagccaaggtgaaggaggagatcccagacccagatgtcaagggatctccccacctcagtgacagtgcctgcctggggcagcaaagggaaggaggagggacagagctagtggggaccatgatgacgtctaacactccagagaggactagccagggaggggctggcgtctcgcctttgctggtgaaggaggaacccaaggaagataacggcctgcccacctcctttactttgaatgctgccgacaggcccgccaaccacacaaagctgaaagacccctccgagtatgtggccaacagtgcgtcagcattgttcagccaggacatctctgttaagatggcgtctgattttctcatgaagctgtcagctgcaaatcagaaggagcccatgaatcttaattttaaagtgaaagaggagcctaaggaaggggagtccctaagcacgactttgcctcggtccagctatgtgttcagcccggaatctgaagtgtcagccccaggcgtctctgaggacgcactaaagccccaggaagggaagggaagtgtgctaaggcgggatgtgtcagtcaaagcagcctctgaacttctcatgaaactctcagcggaaagctacaaggaaacacagatggtgaagattaaagaggaacccatggaggttgacatccaggactcccatgtctcgatatcacccagccggaatgttggctacagcactttaatcgggcgagagaaaaccgaacccttacagaagatgccagagggcagagtacccccagagagaaacctcttcagtcaggatatctctgtgaaaatggcttccgagctcctctttcaactgtcagaaaaagtgagcaaagagcacaatcatacaaaagaaaacaccatccggaccacgaccagccctttcttttcagaagacacatttagacaatcaccattcacctccaattcaaaagaactgctgcccagtgactccgtgctgcacggaagaatatcagctccagaaacagaaaagatagtcctagaggcaggaaatggattaccatcctggaaattcaatgaccagctttttccctgtgacgtgtgtgggaaagtgtttggccgacagcagacattgtcccgacacctctcgctgcacacagaggaaagaaaatacaaatgccacttgtgcccctatgctgctaagtgccgtgcaaatctgaaccagcacttgaccgtccattccgtgaagctggtgagtacagacaccgaggacattgtcagcgccgtcacctctgaaggcagtgatgggaagaaacatccttattattacagttgtcacgtgtgtggatttgagaccgagctcaatgtccagtttgtcagccacatgtcactccacgtggacaaggagcagtggatgttttcgatctgctgcactgcctgcgacttcgtcaccatggaggaagcagagataaagactcacattggcaccaagcacacaggggaagacaggaagacccccagcgaatcaaatagcccctcttcatcctccctctcagctctgagtgattcagccaacagcaaagatgattcagatggctcccagaaaaacaagggcgggaacaatctgctggtcatctctgtcatgcctgggagccagccctcactgaacagtgaggaaaagccagagaaagggttcgaatgtgttttttgcaactttgtctgcaagacgaagaacatgtttgagcgtcatctgcagatacacctcatcacccggatgtttgagtgtgatgtgtgccacaagttcatgaagacccccgaacagctgctggagcataagaaatgccacactgtccccaccggtgggctcaagacgcgtacgcggccgctcgagcagaaactcatctcagaagaggatctggcagcaaatgatatcctggattacaaggatgacgacgataaggtttaa

SEQ ID NO: 17 - DNA, 2709 bp, Artificial Sequence
  ZnF4-9 domain mutant DNA
gcgatcgccatgcccaggaggaagcaggagcagcccaagcgccttccctcacatgttagtaggcaggaagaggcggagggagagctcagtgaaggagaacactggtatggaaactcttcagagactccgtcagaagcatcctatggggaagtccaggagaactataagttgtctctggaggaccggatccaggagcagtccacgtccccggacacctccttgggaagcacgactcccagcagccacacgttggagctggtggcactggacagtgaggtcctgcgagactcactgcagtgtcaagatcacctttccccgggagtgtcttctttgtgtgacgatgacccaggctccaacaagcccctgagcagcaatttgaggcggctgctggaggctggttccctcaaactcgatgctgcagccacggctaatggcagagtggagtcccccgtaaacgttggctcgaacctctccttttccccgccttcccaccacgcccagcagctcagtgttctggccaggaagttggccgagaagcaggaacagaatgaccaatacactccaagtaaccgttttatatggaatcaaggtaagtggttgccaaactcaaccaccacctgcagcttgtctccggattcagccatcctaaaactgaaagctgcagccaatgccgttctgcaggacaaatctctcaccaggactgaggagaccatgcgatttgagtccttttcctccccttttagctcccagtctgccagttctaccttggccgctttgtccaagaaggtcagtgaaagaagtttgactcctggtcaggagcaccctccaccggccagctccttcctttccctggcttctatgacctcctcagcggcccttctgaaggaggtggccgcaagggctgcgggcagtcttctggctgagaaatcatcgctgctgcctgaggaccctctaccgcccccgccttcagagaagaaaccagaaaaagtcactccgccacctccaccgccacctccaccacctccaccaccaccaccacaatccctggaattattattactcccagttcctaagggaagagtttctaaaccctccaattcagcctcagaagaggaaagtggaaagcctttccagtgtccaatatgtggtttggttattaaaaggaagagttactggaagcggcacatggtgattcacacaggtttaaaaagtcatcagtgtccgctctgtccattccggtgtgctcgcaaggacaatctcaaatcccacatgaaggttcatcagcaccaggatcggggagagacctttcagtgccagctgtgcccttttacttcctcacgccacttcagcctgaaactccacatgcgttgccatcagcacttcctgaggacagaagccaaggtgaaggaggagatcccagacccagatgtcaagggatctccccacctcagtgacagtgcctgcctggggcagcaaagggaaggaggagggacagagctagtggggaccatgatgacgtctaacactccagagaggactagccagggaggggctggcgtctcgcctttgctggtgaaggaggaacccaaggaagataacggcctgcccacctcctttactttgaatgctgccgacaggcccgccaaccacacaaagctgaaagacccctccgagtatgtggccaacagtgcgtcagcattgttcagccaggacatctctgttaagatggcgtctgattttctcatgaagctgtcagctgcaaatcagaaggagcccatgaatcttaattttaaagtgaaagaggagcctaaggaaggggagtccctaagcacgactttgcctcggtccagctatgtgttcagcccggaatctgaagtgtcagccccaggcgtctctgaggacgcactaaagccccaggaagggaagggaagtgtgctaaggcgggatgtgtcagtcaaagcagcctctgaacttctcatgaaactctcagcggaaagctacaaggaaacacagatggtgaagattaaagaggaacccatggaggttgacatccaggactcccatgtctcgatatcacccagccggaatgttggctacagcactttaatcgggcgagagaaaaccgaacccttacagaagatgccagagggcagagtacccccagagagaaacctcttcagtcaggatatctctgtgaaaatggcttccgagctcctctttcaactgtcagaaaaagtgagcaaagagcacaatcatacaaaagaaaacaccatccggaccacgaccagccctttcttttcagaagacacatttagacaatcaccattcacctccaattcaaaagaactgctgcccagtgactccgtgctgcacggaagaatatcagctccagaaacagaaaagatagtcctagaggcaggaaatggattaccatcctggaaattcaatgaccagctttcaaatagctcttcatcctccctctcagctctgagtgattcagccaacagcaaagatgattcagatggctcccagaaaaacaagggcgggaacaatctgctggtcatctctgtcatgcctgggagccagcccactgtccccaccggtgggctcaagacgcgtacgcggccgctcgagcagaaactcatctcagaagaggatctggcagcaaatgatatcctggattacaaggatgacgacgataaggtttaa

SEQ ID NO: 18 - DNA, 1494 bp, Artificial Sequence
  ZnF1-9 domain mutant DNA
gcgatcgccatgcccaggaggaagcaggagcagcccaagcgccttccctcacatgttagtaggcaggaagaggcggagggagagctcagtgaaggagaacactggtatggaaactcttcagagactccgtcagaagcatcctatggggaagtccaggagaactataagttgtctctggaggaccggatccaggagcagtccacgtccccggacacctccttgggaagcacgactcccagcagccacacgttggagctggtggcactggacagtgaggtcctgcgagactcactgcagtgtcaagatcacctttccccgggagtgtcttctttgtgtgacgatgacccaggctccaacaagcccctgagcagcaatttgaggcggctgctggaggctggttccctcaaactcgatgctgcagccacggctaatggcagagtggagtcccccgtaaacgttggctcgaacctctccttttccccgccttcccaccacgcccagcagctcagtgttctggccaggaagttggccgagaagcaggaacagaatgaccaatacactccaagtaaccgttttatatggaatcaaggtaagtggttgccaaactcaaccaccacctgcagcttgtctccggattcagccatcctaaaactgaaagctgcagccaatgccgttctgcaggacaaatctctcaccaggactgaggagaccatgcgatttgagtccttttcctccccttttagctcccagtctgccagttctaccttggccgctttgtccaagaaggtcagtgaaagaagtttgactcctggtcaggagcaccctccaccggccagctccttcctttccctggcttctatgacctcctcagcggcccttctgaaggaggtggccgcaagggctgcgggcagtcttctggctgagaaatcatcgctgctgcctgaggaccctctaccgcccccgccttcagagaagaaaccagaaaaagtcactccgccacctccaccgccacctccaccacctccaccaccaccaccacaatccctggaattattattactcccagttcctaagggaagagtttctaaaccctccaattcagcctcagaagaggaaagtggaaagcctcagcacttcctgaggacagaagccaaggtgaaggaggagatcccagacccagatgtcaagggatctccccacctcagtgacagtgcctgcctggggcagcaaagggaaggaggagggacagagctagtggggaccatgatgacgtctaacactccagagaggactagccagggaggggctggcgtctcgcctttgctggtgaaggaggaacccaaggaagataacggcctgcccacctcctttactttgaatgctgccgacaggcccgccacgcgtacgcggccgctcgagcagaaactcatctcagaagaggatctggcagcaaatgatatcctggattacaaggatgacgacgataaggtttaa

SEQ ID NO: 19 - DNA, 3336 bp, Artificial Sequence
  ZnF sumo domain mutant DNA
gcgatcgccatgcccaggaggaagcaggagcagcccaagcgccttccctcacatgttagtaggcaggaagaggcggagggagagctcagtgaaggagaacactggtatggaaactcttcagagactccgtcagaagcatcctatggggaagtccaggagaactataagttgtctctggaggaccggatccaggagcagtccacgtccccggacacctccttgggaagcacgactcccagcagccacacgttggagctggtggcactggacagtgaggtcctgcgagactcactgcagtgtcaagatcacctttccccgggagtgtcttctttgtgtgacgatgacccaggctccaacaagcccctgagcagcaatttgaggcggctgctggaggctggttccctcaaactcgatgctgcagccacggctaatggcagagtggagtcccccgtaaacgttggctcgaacctctccttttccccgccttcccaccacgcccagcagctcagtgttctggccaggaagttggccgagaagcaggaacagaatgaccaatacactccaagtaaccgttttatatggaatcaaggtaagtggttgccaaactcaaccaccacctgcagcttgtctccggattcagccatcctaaaactgaaagctgcagccaatgccgttctgcaggacaaatctctcaccaggactgaggagaccatgcgatttgagtccttttcctccccttttagctcccagtctgccagttctaccttggccgctttgtccaagaaggtcagtgaaagaagtttgactcctggtcaggagcaccctccaccggccagctccttcctttccctggcttctatgacctcctcagcggcccttctgaaggaggtggccgcaagggctgcgggcagtcttctggctgagaaatcatcgctgctgcctgaggaccctctaccgcccccgccttcagagaagaaaccagaaaaagtcactccgccacctccaccgccacctccaccacctccaccaccaccaccacaatccctggaattattattactcccagttcctaagggaagagtttctaaaccctccaattcagcctcagaagaggaaagtggaaagcctttccagtgtccaatatgtggtttggttattaaaaggaagagttactggaagcggcacatggtgattcacacaggtttaaaaagtcatcagtgtccgctctgtccattccggtgtgctcgcaaggacaatctcaaatcccacatgaaggttcatcagcaccaggatcggggagagacctttcagtgccagctgtgcccttttacttcctcacgccacttcagcctgaaactccacatgcgttgccatcagcacttcctgaggacagaagccaaggtgagagaggagatcccagacccagatgtcaagggatctccccacctcagtgacagtgcctgcctggggcagcaaagggaaggaggagggacagagctagtggggaccatgatgacgtctaacactccagagaggactagccagggaggggctggcgtctcgcctttgctggtgagagaggaacccaaggaagataacggcctgcccacctcctttactttgaatgctgccgacaggcccgccaaccacacaaagctgaaagacccctccgagtatgtggccaacagtgcgtcagcattgttcagccaggacatctctgttaagatggcgtctgattttctcatgaagctgtcagctgcaaatcagaaggagcccatgaatcttaattttaaagtgagagaggagcctaaggaaggggagtccctaagcacgactttgcctcggtccagctatgtgttcagcccggaatctgaagtgtcagccccaggcgtctctgaggacgcactaaagccccaggaagggaagggaagtgtgctaaggcgggatgtgtcagtcaaagcagcctctgaacttctcatgaaactctcagcggaaagctacaaggaaacacagatggtgaagattagagaggaacccatggaggttgacatccaggactcccatgtctcgatatcacccagccggaatgttggctacagcactttaatcgggcgagagagaaccgaacccttacagaagatgccagagggcagagtacccccagagagaaacctcttcagtcaggatatctctgtgaaaatggcttccgagctcctctttcaactgtcagaaaaagtgagcaaagagcacaatcatacaaaagaaaacaccatccggaccacgaccagccctttcttttcagaagacacatttagacaatcaccattcacctccaattcaaaagaactgctgcccagtgactccgtgctgcacggaagaatatcagctccagaaacagaaaagatagtcctagaggcaggaaatggattaccatcctggaaattcaatgaccagctttttccctgtgacgtgtgtgggaaagtgtttggccgacagcagacattgtcccgacacctctcgctgcacacagaggaaagaaaatacaaatgccacttgtgcccctatgctgctaagtgccgtgcaaatctgaaccagcacttgaccgtccattccgtgaagctggtgagtacagacaccgaggacattgtcagcgccgtcacctctgaaggcagtgatgggaagaaacatccttattattacagttgtcacgtgtgtggatttgagaccgagctcaatgtccagtttgtcagccacatgtcactccacgtggacaaggagcagtggatgttttcgatctgctgcactgcctgcgacttcgtcaccatggaggaagcagagataaagactcacattggcaccaagcacacaggggaagacaggaagacccccagcgaatcaaatagcccctcttcatcctccctctcagctctgagtgattcagccaacagcaaagatgattcagatggctcccagaaaaacaagggcgggaacaatctgctggtcatctctgtcatgcctgggagccagccctcactgaacagtgaggaaaagccagagaaagggttcgaatgtgttttttgcaactttgtctgcaagacgaagaacatgtttgagcgtcatctgcagatacacctcatcacccggatgtttgagtgtgatgtgtgccacaagttcatgaagacccccgaacagctgctggagcataagaaatgccacactgtccccaccggtgggctcaagacgcgtacgcggccgctcgagcagaaactcatctcagaagaggatctggcagcaaatgatatcctggattacaaggatgacgacgataaggtttaa

SEQ ID NO: 20 - DNA, 23 bp, Artificial Sequence
  Guide RNA sequence
gtctctggaggaccggatccagg

SEQ ID NO: 21 - DNA, 18 bp, Artificial Sequence
  ss telomeric G rich oligo
ttagggttagggttaggg

SEQ ID NO: 22 - DNA, 18 bp, Artificial Sequence
  ss telomeric C rich oligo
ccctaaccctaaccctaa

SEQ ID NO: 23 - DNA, 100 bp, Artificial Sequence
  ss pentaprobe 3
cgctctattctactgtcctgtgcattcaatcgttgagttcgatctagtctcgtctaaccctcccctgctccgctggtctggcctcgcctatcctacccat

SEQ ID NO: 24 - DNA, 100 bp, Artificial Sequence
  ss pentaprobe 9
atgggtaggataggcgaggccagaccagcggagcaggggagggttagacgagactagatcgaactcaacgattgaatgcacaggacagtagaatagagcg

SEQ ID NO: 25 - DNA, 17 bp, Artificial Sequence
  Fluorophore conjugated centromere-telomere PNA probe
aaactagacagaagcat

SEQ ID NO: 26 - protein, 270 aa, Homo sapiens
MWNSGFESYGSSSYGGAGGYTQSPGGFGSPAPSQAEKKSRARAQHIVPCTISQLLSATLVDEVFRIGNVEISQVTIVGIIRHAEKAPTNIVYKIDDMTAAPMDVRQWVDTDDTSSENTVVPPETYVKVAGHLRSFQNKKSLVAFKIMPLEDMNEFTTHILEVINAHMVLSKANSQPSAGRAPISNPGMSEAGNFGGNSFMPANGLTVAQNQVLNLIKACPRPEGLNFQDLKNQLKHMSVSSIKQAVDFLSNEGHIYSTVDDDHFKSTDAE

SEQ ID NO: 27 - protein, 121 aa, Homo sapiens
MVDMMDLPRSRINAGMLAQFIDKPVCFVGRLEKIHPTGKMFILSDGEGKNGTIELMEPLDEEISGIVEVVGRVTAKATILCTSYVQFKEDSHPFDLGLYNEAVKIIHDFPQFYPLGIVQHD

(19) United States
(12) Patent Application Publication
(10) Pub. No.: US 2022/0305003 A1
(43) Pub. Date: Sep. 29, 2022

(54) METHODS OF TREATING CANCER WITH AN INHIBITOR OF ZNF827

(71) Applicant: CHILDREN'S MEDICAL RESEARCH INSTITUTE, Westmead, New South Wales (AU)

(72) Inventor: Hilda PICKETT, Westmead, New South Wales (AU)

(21) Appl. No.: 17/616,861

(22) PCT Filed: Jun. 12, 2020
(86) PCT No.: PCT/AU2020/050594

§ 371 Date: Dec. 6, 2021

(30) Foreign Application Priority Data
Jun. 14, 2019 (AU) 2019902078

(51) Int. Cl. A61K 31/4745; A61P 35/00; A61K 31/047; A61K 31/519; A61K 31/138; A61K 31/4439; A61K 31/454; A61K 31/502; A61K 31/55; A61K 31/192; A61K 31/506; A61K 31/155; C12N 15/113

(52) U.S. Cl. CPC A61K 31/4745; A61P 35/00; A61K 31/047; A61K 31/519; A61K 31/138; A61K 31/4439; A61K 31/454; A61K 31/502; A61K 31/55; A61K 31/192; A61K 31/506; A61K 31/155; C12N 15/1136; C12N 2310/14

(57) ABSTRACT

The present disclosure relates to inhibitors of ZNF827 and their use alone or in combination with anti-cancer agents to treat cancer. Methods of selecting a subject for treatment with an inhibitor of ZNF827 are also detailed.

### CROSS-REFERENCE TO RELATED APPLICATIONS

The present application claims priority from Australian Provisional Patent Application No 2019902078 filed on 14 Jun. 2019, the content of which is incorporated herein by reference in its entirety.

### TECHNICAL FIELD

The present disclosure generally relates to cancer and methods of treating cancer.

### BACKGROUND

Cancer remains a leading cause of mortality in Australia (and worldwide), with an estimated 138,000 new cases and approximately 48,500 deaths in Australia in 2018. Genome instability, which collectively refers to genetic aberrations of the genome, is a hallmark of cancer cells and a mechanism of carcinogenesis. A major source of genome instability arises from defective DNA damage response (DDR) and DNA repair pathways. Germline mutations in DDR and DNA repair genes often underlie cancer predisposition and various cancers contain genetic defects in these pathways, thus limiting their DNA repair capacity. Consequently, DNA repair pathway components have attracted interest as targets for novel and specific cancer treatments, both independently and in the context of synthetic lethality, whereby simultaneous perturbations of two genes result in cell death. The success of synthetic lethality-based anticancer therapeutics is highlighted by the recent registration of poly(ADP-ribose) polymerase (PARP) inhibitors to treat homologous recombination (HR) deficient breast and ovarian cancers with BRCA1/2 mutations.

DNA double strand breaks (DSBs) represent the most catastrophic form of DNA lesions. The two major mechanisms of DSB repair are HR and non-homologous end joining (NHEJ). HR is the most error-free and reliable repair pathway, but relies upon DNA end resection to generate single-stranded DNA (ssDNA) intermediates that are necessary for homology-directed repair. ssDNA is unstable and subject to the formation of secondary structures, as well as being prone to chemical and nucleolytic attack. The protection of ssDNA and the correct triage of ssDNA into the appropriate pathway for DNA synthesis or repair is important to the maintenance of genome stability.

Telomeres are nucleoprotein structures that function to cap the ends of linear chromosomes, preventing them from being recognised as DNA DSBs. Telomere dysfunction results in the engagement of DSB repair pathways. Various proteins have been identified as playing one or more roles in such repair pathways. The zinc finger protein ZNF827 is a largely uncharacterized protein which has been implicated in DNA repair through its posited association with the Nucleosome Remodelling (NuRD) complex at ALT telomeres (Conomos et al., 2014). Conomos et al., 2014 suggests a role for ZNF827 in the engagement of HR at telomeres, potentially via the recruitment of NuRD to telomeres. However, the precise role of ZNF827 in the DNA damage and repair pathway has not been elucidated.

### SUMMARY

The present disclosure is based on the surprising finding that ZNF827 has a role beyond its previously suggested association with telomeres, in the global DDR. The inventors have shown that ZNF827 binds directly to ssDNA and interacts with two key effector molecules in the DDR, replication protein A (RPA) and topoisomerase 2-binding protein 1 (TOPBP1). The inventors have also shown that inhibition of ZNF827 can reduce cell proliferation and induce apoptosis of cancer cells. Furthermore, the inventors have shown that inhibition of ZNF827 in conjunction with the administration of an anti-cancer agent can produce a synergistic effect in reducing cell proliferation and inducing an apoptotic response. On the basis of these findings, the inventors have developed new methods of inhibiting cancer cell viability and/or growth, methods of sensitizing cancer cells to therapy with an anti-cancer agent, and methods of treating cancer, comprising inhibiting ZNF827.

Accordingly, in one aspect the present disclosure provides a method of treating cancer, comprising inhibiting ZNF827 in a subject in need thereof, wherein the subject is receiving simultaneous, separate or sequential treatment with an anti-cancer agent.

The methods disclosed herein may comprise inhibiting cancer cell viability and/or growth.

In another aspect, the present disclosure provides a method of sensitizing a cancer cell to an anti-cancer agent, comprising inhibiting ZNF827. Thus, the present disclosure provides a method of sensitizing a cancer cell to an anti-cancer agent, comprising administering an inhibitor of ZNF827 to the cancer cell. The inhibitor of ZNF827 may be any such inhibitor disclosed herein.

The anti-cancer agent may be a DNA-damaging agent.

Alternatively or in addition, the anti-cancer agent may be an alkylating agent, an antimetabolite, an anti-tumour antibiotic, a topoisomerase inhibitor, a mitotic inhibitor, a corticosteroid or a PARP inhibitor.

Alternatively or in addition, the anti-cancer agent is selected from the group consisting of: irinotecan, camptothecin, etoposide, teniposide, doxorubicin, olaparib, rucaparib, niraparib, AKT inhibitor VIII, Axitinib, AZ628, Bexarotene, CI-1040, FMK, FR-180204, GW441756, I-BET-762, Imatinib, KIN001-236, KIN001-244, KIN001-260, Nilotinib, NPK76-II-72-1, NVP-BHG712, OSI-930, PD0325901, Phenformin, SNX-2112, Sunitib, T0901317, TAK-715, Tamoxifen, THZ-2-102-1, THZ-2-49, TL-1-85, TL-2-105, Tretinoin, VNLG-124, Vorinostat and VX-702 and topotecan.

The anti-cancer agent may be topotecan.

In another aspect, the present disclosure provides a method of treating cancer, comprising inhibiting ZNF827 in a subject in need thereof, wherein the cancer is not an ALT cancer. Any of the methods of identifying or detecting an ALT cancer cell disclosed herein may be used in this regard.

Any of the methods disclosed herein my comprise inhibiting ZNF827 by administering an inhibitor of ZNF827. The inhibitor can be any one or more of a genetic inhibitor, a small molecule, a peptide and a protein.

In one embodiment, the inhibitor is a genetic inhibitor. For example, the genetic inhibitor may be siRNA.

Any of the methods disclosed herein may comprise inhibiting ZNF827 by disrupting its binding to one or more of its endogenous binding partner at a ZNF827 binding domain. For example, inhibiting ZNF827 may comprise disrupting the binding of ZNF827 to an endogenous binding partner at the N-terminal RRK motif of ZNF827. In another example, inhibiting ZNF827 comprises disrupting the binding of ZNF827 to an endogenous binding partner at any one or more zinc finger domain of ZNF827. In yet another example, inhibiting ZNF827 comprises disrupting the binding of ZNF827 to an endogenous binding partner at any one or more SUMOylation site of ZNF827.

In another aspect, the present disclosure provides a method of selecting a subject for treatment with an inhibitor of ZNF827, the method comprising determining the level of expression and/or activity of ZNF827 in the subject, wherein if the level of expression and/or activity of ZNF827 in the subject is normal, the subject is selected for treatment with the inhibitor of ZNF827.

In another aspect, the present disclosure provides a method of selecting a subject for treatment with an anti-cancer agent, the method comprising determining the level of expression and/or activity of ZNF827 in the subject, wherein if the level of expression and/or activity of ZNF827 in the subject is low, the subject is selected for treatment with the anti-cancer agent.

In another aspect, the present disclosure provides a method of selecting a subject for treatment with an anti-cancer agent and an inhibitor of ZNF827, the method comprising determining the level of expression and/or activity of ZNF827 in the subject, wherein if the level of expression and/or activity of ZNF827 in the subject is normal, the subject is selected for treatment with the anti-cancer agent and the inhibitor of ZNF827.

In another aspect, the present disclosure provides a method of predicting the response of a subject to an anti-cancer agent, the method comprising determining the level of expression and/or activity of ZNF827 in the subject, wherein a low level of expression and/or activity of ZNF827 in the subject is indicative that the subject's response to the anti-cancer agent is likely improved relative to if the subject had a normal level of expression of ZNF827.

In another aspect, the present disclosure provides a method of identifying whether a subject suffering from cancer is suitable for treatment with an inhibitor of ZNF827, the method comprising determining the level of expression and/or activity of ZNF827 in the subject, wherein if the level of expression and/or activity of ZNF827 in the subject is normal, the subject is identified as being suitable for treatment with the inhibitor of ZNF827.

In another aspect, the present disclosure provides a method of identifying whether a subject suffering from cancer is suitable for treatment with an anti-cancer agent, the method comprising determining the level of expression and/or activity of ZNF827 in the subject, wherein if the level of expression and/or activity of ZNF827 in the subject is low, the subject is identified as being suitable for treatment with the anti-cancer agent.

In another aspect, the present disclosure provides a pharmaceutical composition comprising an inhibitor of ZNF827 and an anti-cancer agent. The pharmaceutical composition may be for use in treating cancer.

In another aspect, the present disclosure provides a method of preparing the pharmaceutical composition disclosed herein, comprising combining an inhibitor of ZNF827 and an anti-cancer agent.

In another aspect, the present disclosure provides a pharmaceutical composition comprising an inhibitor of ZNF827 for use in treating cancer, wherein the cancer is not an ALT cancer.

The pharmaceutical composition may consist essentially of an inhibitor of ZNF827.

In another aspect, the present disclosure provides the use of an inhibitor of ZNF827 and an anti-cancer agent in the manufacture of a medicament for the treatment of cancer.

In another aspect, the present disclosure provides a use of an inhibitor of ZNF827 in the manufacture of a medicament for the treatment of cancer, wherein the cancer is not an ALT cancer.

The medicament may consist essentially of an inhibitor of ZNF827.

The anti-cancer agent may be a DNA-damaging agent.

Alternatively or in addition, the anti-cancer agent may be an alkylating agent, an antimetabolite, an anti-tumour antibiotic, a topoisomerase inhibitor, a mitotic inhibitor, a corticosteroid or a PARP inhibitor.

Alternatively or in addition, the anti-cancer agent is selected from the group consisting of: irinotecan, camptothecin, etoposide, teniposide, doxorubicin, olaparib, rucaparib, niraparib, AKT inhibitor VIII, Axitinib, AZ628, Bexarotene, CI-1040, FMK, FR-180204, GW441756, I-BET-762, Imatinib, KIN001-236, KIN001-244, KIN001-260, Nilotinib, NPK76-II-72-1, NVP-BHG712, OSI-930, PD0325901, Phenformin, SNX-2112, Sunitib, T0901317, TAK-715, Tamoxifen, THZ-2-102-1, THZ-2-49, TL-1-85, TL-2-105, Tretinoin, VNLG-124, Vorinostat and VX-702 and topotecan.

The anti-cancer agent may be topotecan.

The inhibitor of ZNF827 may be any one or more of a genetic inhibitor, a small molecule, a peptide and a protein.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1. ZNF827 and mutants Schematic of ZNF827 full length, ZNF827 mutant with ZnF1-3 deleted (ZnF1-3 deleted), ZNF827 mutant with ZnF4-9 deleted (ZnF4-9 deleted), ZNF827 mutant with no zinc fingers (ZnF1-9 deleted), and ZNF827 SUMO mutant. Empty bars indicate missing or mutated regions in the mutants. Regions of interest in ZNF827 are annotated based on experimental results.

FIG. 2. ZNF827 displays preferential binding to ssDNA in a non-sequence-specific manner (a) Electrophoretic mobility shift assays (EMSAs) showing binding of ZNF827 to ss telomeric repeats and no binding activity to ds telomeric repeats (left panel). The binding of ZNF827 to ss telomeric repeats requires ZnF1-3 (right panel). (b) EMSAs showing binding of ZNF827 to ss non-telomeric DNA and no binding activity to ds non-telomeric DNA (top panel). The binding of ZNF827 to ss non-telomeric repeats requires ZnF1-3 (middle panel). ZNF827 and mutants display no binding activity to ds DNA (bottom panel). FIG. 3. ZNF827 interacts directly with RPA and TOPBP1 (a) ZNF827-RPA interaction demonstrated by ZNF827 (red, leftmost column) and RPA32 (green, second column from the left, a component of the heterotrimeric RPA complex) colocalisations by immunofluorescence (IF) (top panel) and co-immunoprecipitation with ZNF827 antibody (bottom panel). (b) Co-immunoprecipitation with ZNF827 antibody showing that deletion of the ZnF1-3 or, to a lesser extent, ZnF4-9 zinc finger cluster disrupts the ZNF827-RPA interaction. (c) Co-immunoprecipitation with Myc antibody demonstrating a direct interaction between ZNF827 and TOPBP1, and that their interaction does not require either zinc finger cluster, but is dependent on the NuRD-binding RRK motif , and the region between the two ZnF clusters (which is also deleted in ZnF1-9 deleted). (d) ZNF827-RPA interaction shown by co-immunoprecipitation is abolished in benzonase (a nuclease that digests DNA)-treated samples, indicating that the presence of DNA is required for the interaction.

FIG. 4. ZNF827 depletion exacerbates RPA foci induction by topotecan. (a) Representative images of colocalisations of Myc-tagged ZNF827 and the mutants (red, leftmost column) with RPA2 (green, second column from the left) labelled by IF following 48 hr overexpression of the Myc-tagged ZNF827 and mutant constructs in ZNF827 KO U-2 OS cells. Nuclei stained by DAPI in blue. (b) Representative images of RPA2 foci (red, leftmost column) labelled by IF and telomeres (green, second column from the left) labelled by FISH in U-2 OS and HT1080 following 72 hr siRNA knockdown of ZNF827 and 24 hr incubation with 2 μg/mL topotecan or DMSO. Nuclei stained by DAPI in blue. (c) Data quantitation of the frequency of RPA2 colocalising with telomeres (top) and the total nuclear RPA foci (bottom) by automated counting using CellProfiler. Data presented as mean±SEM from 200 nuclei per experimental condition in one replicate; ***P<0. 0002, ****P<0.0001 by two tailed t test.

FIG. 5. ZNF827 is involved in the DNA damage and repair pathway. (a) Representative images of γH2AX (red, second column from the left) and ZNF827 (green, leftmost column) colocalisations labelled by IF in WI38-VA13/2RA cells overexpressing ZNF827, harvested 1 hr post IR exposure (top panel). Nuclei stained by DAPI in blue. Quantitation of γH2AX and ZNF827 colocalisations from 50 nuclei per experimental condition by manual counting (bottom panel). Data presented as mean±SEM from one replicate; **P<0.005 by two tailed t test. (b) Comet assay showing a representative image of comet tails from each experimental condition in U-2 OS cells following 72 hr siRNA knockdown of ZNF827 and 24 hr incubation with 2 μg/mL topotecan or DMSO (left panel). Quantitation of tail moment in 50 cells per experimental condition by a semi-automated approach using CellProfiler (right panel). Data presented as mean±SEM from one replicate; *P<0.05, **P<0.005 by two tailed t test. (c) Quantitative data of ZNF827 and telomere colocalisations in WI38-VA13/2RA overexpressing ZNF827, harvested at 0.5, 1, 1.5, 2 and 3 hr post IR exposure. Data presented as mean±SEM from 236 nuclei per experimental condition from one replicate; **P<0.005 by two tailed t test.

FIG. 6. ZNF827 interacts with ATR. (a) Representative images of ZNF827 (red, leftmost column) and ATR (green, second column from the left) colocalisations labelled by IF in U-2 OS cells following 48 hr ZNF827 overexpression and 24 hr incubation with 2 μg/mL topotecan or DMSO. Nuclei stained by DAPI in blue. (b) Representative images of PLA assay showing interaction between ZNF827 and ATR. (c) Western blot analysis of p-CHK1 (S345), total CHK1, p-RPA2 (S33), total RPA2 and pCHK2 (T68) in U-2 OS cells following 72 hr siRNA knockdown of ZNF827 and 24 hr incubation with 2 μg/mL topotecan or DMSO. Vinculin used as a loading control. (d) Western blot analysis of p-CHK1 (S345) and total CHK1 in U-2 OS cells following 72 hr siRNA knockdown of ZNF827 and 20 hr incubation with 1 μM aphidicolin or DMSO. Vinculin used as a loading control.

FIG. 7. ZNF827 localises to replication forks and plays a role in the cellular response to replication stress. (a) Representative images of two PCNA (red, leftmost column) nuclear localisation patterns, and colocalisations between ZNF827 (green, second column from the left) and PCNA (red, leftmost column) labelled by IF in U-2 OS and HT1080 following 48 hr ZNF827 and PCNA chromobody (Cell Cycle Chromobody®-RFP) overexpression and 6 hr incubation with 0.4 μM aphidicolin to induce stalled replication forks. Nuclei stained by DAPI in blue (third column from the left). (b) Representative images of γH2AX (red, first row) and endogenous ZNF827 (green, second row) foci labelled by IF in HT1080 and HT1080 6TG following 24 hr incubation with 2 μg/mL topotecan or DMSO (top panel). Nuclei stained by DAPI in blue (third row). (c) Data quantitation of γH2AX foci induction (bottom panel, left), ZNF827 foci induction (bottom panel, middle) and γH2AX and ZNF827 colocalisations (bottom panel, right) from 50 nuclei per experimental condition by manual counting. Data presented as mean±SEM from one replicate; ****P<0.0001 by two tailed t test.

FIG. 8. ZNF827 affects HR-directed DNA repair. (a) ZNF827 depletion increases telomere replication stress, as measured by fragile telomeres (left panel), telomere signal free ends (right panel), and chromosome breakage events (bottom panel). Quantitation of frequency of fragile telomeres per chromosome was performed on 950 chromosomes per experimental condition with data presented as mean±SEM. Quantitation of frequency of telomere signal free ends per chromosome was performed on 950 chromosomes per experimental condition with data presented as mean±SEM. Quantitation of frequency of chromosome breakage per chromosome was performed on 950 chromosomes per replicate from two biological replicates with data presented as mean±SEM. *P<0.05, ***P<0.0002 by two tailed t test. (b) ZNF827 depletion impedes DNA repair by HR, as demonstrated by decreased sister chromatid exchanges (SCEs). Quantitative data of SCEs in U-2 OS and U-2 OS ZNF827 CRISPR KO cells incubated with 2 μg/mL topotecan or DMSO for 1 hr (left), and in HT1080 cells incubated with 2 μg/mL topotecan or DMSO for 1 hr at 24 hr within the 72 hr siRNA knockdown of ZNF827. Data presented as mean±SEM from 800-1000 chromosomes per replicate from three biological replicates. *P<0.05, ***P<0.0002, ****P<0.0001 by two tailed t test.

FIG. 9. ZNF827 affects cell cycle progression and ZNF827 depletion causes G1 to early S arrest. (a) Histogram of cell cycle analysis by flow cytometry displaying the cell cycle profile of HT1080 cells following 72 hr siRNA knockdown of ZNF827 and 1 hr incubation with 2 μg/mL topotecan or DMSO. (b) Data quantitation of cell cycle profile of a. (c) Schematic of the fluorescent cellular changes associated with FUCCI cell cycle sensor. Cells in G1 express Cdt1-RFP (red). Cells in G2/M express Geminin-GFP (green). Cells in S phase appear yellow (both Geminin-GFP and Cdt1-RFP expressed). (d) Quantitative data of live cell imaging showing G1 duration in HT1080 FUCCI cells following 72 hr siRNA knockdown of ZNF827 and 1 hr incubation with 2 μg/mL topotecan or DMSO. Data presented as mean±SEM from 40 cells per replicate from three biological replicates; **P<0.005, ***P<0.0002, ****P<0.0001 by two tailed t test. (e) Quantitative data of live cell imaging showing the proportion of cells with G1 duration >6.8 hr in HT1080 FUCCI cells following 72 hr siRNA knockdown of ZNF827 and 1 hr incubation with 2 μg/mL topotecan or DMSO. Data presented as mean±SEM from 40 cells per replicate from three biological replicates; *P<0.05, **P<0.005, by two tailed t test. (f) Quantitative data of live cell imaging showing S/G2-M duration in HT1080 FUCCI cells following 72 hr siRNA knockdown of ZNF827 and 1 hr incubation with 2 μg/mL topotecan or DMSO. Data presented as mean±SEM from 40 cells per replicate from three biological replicates; ***P<0.0002, ****P<0.0001 by two tailed t test.

FIG. 10. ZNF827 depletion suppresses ATR-CHK1 activation, and upregulates p21. Western blot analysis of CHK1 (the main downstream effector of ATR), pCHK1 (activated CHK1), RPA32 (another downstream target of ATR), pRPA32 (activated RPA32), p21, p53 and PARP, following ZNF827 knockdown and topotecan and/or ATR inhibitor treatment as indicated. Western blot analysis of CHK1 (the main downstream effector of ATR) and pCHK1 (activated CHK1) following ZNF827 knockdown and topotecan treatment.

FIG. 11. ZNF827 depletion triggers G1/S arrest, retards cell growth and induces apoptosis synergistically with topotecan. (a) Cell cycle analysis by EdU flow cytometry showing ZNF827 depletion triggers G1/S cell cycle arrest. (b) Live cell growth assays showing that ZNF827 depletion leads to cell growth retardation comparable to topotecan treatment and displays an synergistic growth inhibitory effect with topotecan. (c) Live cell growth assays showing that ZNF827 depletion induces apoptosis synergistically with topotecan treatment.

FIG. 12. Analysis of drug sensitivity for 266 compounds across 660 cell lines in relation to ZNF827 gene expression levels. (a) Histogram of ZNF827 gene expression determined by RNA-seq for a panel of 660 cell lines. Gene expression data was sourced from DepMap Public 19Q2, with values represented as log₂(TPM). TPM=Transcripts per kilobase million. Cell lines with gene expression values: less than 1 were classified as “low” (orange, ‘*’), greater than 5 as “high” (brown, ‘***’), and the rest as “normal” (blue, ‘**’). (b) Box plots of drug sensitivity for the panel of cell lines across 32 drugs that were found to be significantly different (adjusted p-value<0.05) between “low” (orange, left box plot) and “normal” (blue, right box plot) ZNF827 expression groups. Data was sourced from Sanger GDSC v17.3, with drug sensitivity values represented as the natural log of the IC50. Adjusted p-values were calculated using the Nemenyi test followed by FDR correction.

FIG. 13. ZNF827 depletion increases sensitivity to topotecan treatment. Dose-response curves showing U-2 OS cell proliferation when treated with scrambled (open dots) or siZNF827 (solid dots) and topotecan at 0, 0.0039, 0.0078, 0.0156, 0.03125, 0.0625, 0.125, 0.25, 0.5 and 1 μg/mL (presented in log scale) in triplicates. Data presented as mean±SEM from triplicates. IC50 analysis was performed using GraphPad Prism 8.

### KEY TO THE SEQUENCE LISTING

SEQ ID NO: 1 Amino acid sequence for a reference human ZNF827 protein (Uniprot accession no. Q17R98).

SEQ ID NO: 2 Amino acid sequence for a reference human replication protein A subunit 1 (RPA1) (Uniprot accession no. P27694).

SEQ ID NO: 3 Amino acid sequence for a reference human topoisomerase 2-binding protein 1 (TOPBP1) (Uniprot accession no. Q92547).

SEQ ID NO: 4 Amino acid sequence for a reference human ZNF827 protein ZnF1-3 domain.

SEQ ID NO: 5 Amino acid sequence for a reference human ZNF827 protein ZnF4-9 domain.

SEQ ID NO: 6 Amino acid sequence for TOPBP1 binding site on ZNF827.

SEQ ID NO: 7 Nucleotide sequence for a reference human ZNF827 sequence (Genbank accession no. NM_178835.5).

SEQ ID NO: 8 Nucleotide sequence for a reference human RPA1 subunit sequence (Genbank accession no. NM_002945.5).

SEQ ID NO: 9 Nucleotide sequence for a reference human RPA2 subunit sequence (Genbank accession no. v 002946.5).

SEQ ID NO: 10 Nucleotide sequence for a reference human RPA3 subunit sequence (Genbank accession no. NM_002947.5).

SEQ ID NO: 11 Nucleotide sequence for a reference human TOPBP1 sequence (Genbank accession no. NM_007027.4).

SEQ ID NO: 12 Nucleotide sequence of ZNF827 siRNA.

SEQ ID NO: 13 Nucleotide sequence of ZNF827 siRNA.

SEQ ID NO: 14 Amino acid sequence for a reference human ZNF827 protein N-terminal RRK domain.

SEQ ID NO: 15 Amino acid sequence for a reference human telomerase reverse transcriptase protein (Uniprot accession no. 014746).

SEQ ID NO: 16 Nucleotide sequence for a reference human ZNF827 protein ZnF1-3 domain mutant.

SEQ ID NO: 17 Nucleotide sequence for a reference human ZNF827 protein ZnF4-9 domain mutant.

SEQ ID NO: 18 Nucleotide sequence for a reference human ZNF827 protein ZnF1-9 domain mutant.

SEQ ID NO: 19 Nucleotide sequence for a reference human ZNF827 protein Sumo domain mutant.

SEQ ID NO: 20 Nucleotide sequence for guide RNA used in CRISPR experiments

SEQ ID NO: 21 Nucleotide sequence of single-stranded telomeric G rich oligo.

SEQ ID NO: 22 Nucleotide sequence of single-stranded telomeric C rich oligo.

SEQ ID NO: 23 Nucleotide sequence of single-stranded pentaprobe 3.

SEQ ID NO: 24 Nucleotide sequence of single-stranded pentaprobe 9.

SEQ ID NO: 25 Nucleotide sequence of fluorophore conjugated centromere-telomere PNA probe.

SEQ ID NO: 26 Amino acid sequence for a reference human replication protein A subunit 2 (RPA2) (Uniprot accession no. P15927).

SEQ ID NO: 27 Amino acid sequence for a reference human replication protein A subunit 3 (RPA3) (Uniprot accession no. P35244).

### DETAILED DESCRIPTION

### General Techniques and Definitions

Unless specifically defined otherwise, all technical and scientific terms used herein shall be taken to have the same meaning as commonly understood by one of ordinary skill in the art (e.g. in genomics, immunology, molecular biology, immunohistochemistry, biochemistry, oncology, and pharmacology).

The present disclosure is performed using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition., 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment of the present disclosure may be applied mutatis mutandis to any other aspect or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

### ZNF827

ZNF827 was first identified as an ALT-specific telomere-binding protein by proteomics of isolated chromatin segments (PICh) (Dejardin and Kingston, 2009). The sequence of ZNF827 is publicly available. An exemplary sequence is set forth in SEQ ID NO: 1. Alternative isoforms of ZNF827 have been described. For example, one alternative isoform lacks four amino acids at the carboxy terminus of the isoform described in SEQ ID NO: 1. The ZNF827-telomere interaction has been revealed by proximity-dependent biotin identification (BioID) and dCas9-APEX2 biotinylation at genomic elements by restricted spatial tagging (C-BERST) (Garcia-Expositio et al., 2016 and Gao et al., 2018). ZNF827 has been suggested to play a role in DNA repair through its posited association with the Nucleosome Remodelling (NuRD) complex at ALT telomeres (Conomos et al., 2014).

Surprisingly, the inventors have shown that inhibiting ZNF827 can reduce cell proliferation and induce apoptosis of cancer cells. Furthermore, as disclosed herein, ZNF827 has been shown to interact with ssDNA without preference for telomeric sequences. The present disclosure also demonstrates that there is a direct interaction between ZNF827 and replication protein A (RPA) and topoisomerase 2-binding protein 1 (TOPBP1) and that these interactions are enhanced following treatment with an anti-cancer agent. Accordingly, disclosed herein are new methods of inhibiting cancer cell viability and/or growth, methods of sensitizing cancer cells to therapy with an anti-cancer agent, and methods of treating cancer, comprising inhibiting ZNF827.

### Methods of Inhibiting

Inhibition of ZNF827 may be achieved by any suitable means. The inhibition may be partial or complete. The inhibition of ZNF827 may comprise inhibiting the level of activity and/or expression of ZNF827. The inhibition may be apparent relative to a cell in which ZNF827 has not been inhibited. The extent of the inhibition may be any measurable extent. For example, the extent of the inhibition of ZNF827 expression and/or activity may be distinguishable from the level of expression and/or activity of ZNF827 in a cell in which ZNF827 has not been inhibited. For example, the inhibition may comprise a reduction in the level of activity and/or expression of ZNF827 by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% relative to the level of expression and/or activity in a cell in which ZNF827 has not been inhibited.

The inhibition of ZNF827 may achieve an inhibition of cancer cell viability and/or growth. As used herein, the term “inhibiting cancer cell viability and/or growth” shall be taken to mean hindering, reducing, restraining or preventing cancer cell viability and/or growth. The inhibition may be any reduction relative to a cancer cell in which ZNF827 has not been inhibited. Cell viability and/or growth may be inhibited in any measurable amount. Inhibition of cell viability may be complete or may be partial. Thus, the methods disclosed herein may comprise at least partial inhibition of cancer cell viability and/or growth. For example, cell viability and/or growth may be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% following inhibition of ZNF827.

ZNF827 activity and/or expression may be measured by any suitable means. For example, ZNF827 expression may be measured through qRT PCR, or any of the methods disclosed herein. It will be appreciated that ZNF827 expression levels can provide an indication of its activity in a cell.

It will be understood by a person skilled in the art that ZNF827 expression may be inhibited at the mRNA level or at the protein level. Inhibition may be through upstream or downstream effectors of ZNF827 expression. The inhibitor may be a direct inhibitor of ZNF827 expression or an indirect inhibitor of ZNF827 expression. For example, the inhibitor may bind to ZNF827 to inhibit its function by changing its conformation or by affecting its binding site such that it is no longer able to bind to its binding partners. In another example, the inhibitor may bind to binding partners of ZNF827 to inhibit their function by changing their conformation or by affecting their binding site such that they are no longer able to bind to ZNF827. Thus, the methods disclosed herein may or may not comprise also inhibiting one of RPA and/or TOPBP1. The sequences of RPA and TOPBP1 are publicly available. Exemplary sequences are set forth in SEQ ID NOs: 2 (RPA1), 3 (TOPBP1), 26 (RPA2) and 27 (RPA3). Any inhibitor such as those disclosed herein may be capable of inhibiting ZNF827 such that the endogenous function of ZNF827 is inhibited. Methods of determining binding partners to ZNF827 are known in the art. For example, proteins that bind to ZNF827 may be identified through co-immunoprecipitation, immunofluorescence or proximity ligation assays.

Without wishing to be bound by theory, it is believed that the ZNF827 zinc finger domains (ZnF1-3) are required for ZNF827 to bind ssDNA and that this interaction is required for the binding of RPA. Thus, in one example, the binding domain on ZNF827 may be ZnF1-3. In another example, the binding domain on ZNF827 may be ZnF4-9. Exemplary amino acid sequences of these ZNF827 binding domains are set forth in SEQ ID NOs: 4 (ZnF1-3) and 5 (ZnF4-9). In another example, the binding domain on ZNF827 may be where TOPBP1 binds to ZNF827. An exemplary amino acid sequence of this binding domain is set forth in SEQ ID NO: 6.

In one example, the methods disclosed herein comprise disrupting the binding of ZNF827 to an endogenous binding partner at the N-terminal RRK motif of ZNF827.

In another example, the methods disclosed herein comprise disrupting the binding of ZNF827 to an endogenous binding partner at any one or more zinc finger domain of ZNF827.

In another example, the methods disclosed herein comprise disrupting the binding of ZNF827 to an endogenous binding partner at any one or more SUMOylation site of ZNF827.

The inhibitor may be a genetic inhibitor of ZNF827 or its endogenous binding partner. Methods of designing suitable genetic inhibitors are known in the art. Suitable examples of genetic inhibitors include, but are not limited to, DNA (gDNA, cDNA), RNA (sense RNAs, antisense RNAs, mRNAs, tRNAs, rRNAs, small interfering RNAs (siRNAs), short hairpin RNAs (shRNAs), micro RNAs (miRNAs), small nucleolar RNAs (snoRNAs), small nuclear RNAs (snRNAs), ribozymes, aptamers, DNAzymes, antisense oliogonucleotides, vectors, plasmids, other ribonuclease-type complexes, and mixtures thereof. The gene sequences of ZNF827, RPA and TOPBP1 are publicly available and can be used to design suitable genetic inhibitors by methods known in the art. Reference nucleotide sequences of ZNF827, RPA and TOPBP1 are provided in SEQ ID NOs: 7-11.

Examples of suitable genetic inhibitors are described herein in the experimental examples. Thus, the genetic inhibitors may comprise siRNA inhibitors comprising or consisting of the nucleotide sequences disclosed in SEQ ID NO: 12 or SEQ ID NO: 13. It will be appreciated by the skilled person that inhibition of ZNF827 and/or its endogenous binding partner may also be achieved through knocking out ZNF827 and/or its endogenous binding partner through genome editing (e.g., CRISPR/Cas9, CRISPR/Cas12, etc.).

In one example, the inhibitor is an inhibitor of the ZNF827 protein. The inhibitor may be a small molecule, a peptide or a protein. In one example, the inhibitor is a small molecule. Screens for small molecule inhibitors are known in the art. For example, in Voter et al., 2016, a screen was developed to identify a small molecule inhibitor that disrupts the binding of FANCM to one of its endogenous binding partners. The small molecule may be one which interacts with the ZNF827 protein such that it disrupts the binding of ZNF827 to one or more of its endogenous binding partners. Thus, the small molecule may be selected as one which binds to one or more of the binding domains of ZNF827 as disclosed herein. Alternatively, the small molecule may be one which binds to one or more of ZNF827's endogenous binding partners such that the binding interaction with ZNF827 is disrupted.

The inhibitor may be a peptide mimicking all or part of the binding motifs of ZNF827. In one example, the peptide is a peptide mimicking all or part of the N-terminal RRK motif of ZNF827. For example, the peptide may be a peptide comprising or consisting of an amino acid sequence that is at least 90% identical to the amino acid sequence MPRRKQPQEK (SEQ ID NO: 14). The peptide may be at least 90%, at least 91%, at least 92%, at least 93%, at least 94%, at least 95%, at least 96%, at least 97%, at least 98%, at least 99%, or 100% identical to the amino acid sequence MPRRKQPQEK (SEQ ID NO: 14).

In another example, the peptide is a peptide mimicking all or part of the ZnF1-3 motif of ZNF827 (SEQ ID NO: 4).

In another example, the peptide is a peptide mimicking all or part of the ZnF4-9 motif of ZNF827 (SEQ ID NO: 5).

In another example, the inhibitor is a protein. The peptide or protein may be any peptide capable of binding to ZNF827 and capable of occluding its binding domains. The occlusion may be such that the normal (endogenous) binding interaction between ZNF827 and its endogenous binding partner is disrupted. The peptide or protein may be capable of binding to the ZNF827 binding domains directly or indirectly. Alternatively, the peptide or protein may be any peptide of its endogenous binding partners, capable of binding to ZNF827.

The protein may be a mutant ZNF827 protein that has reduced binding capability to its endogenous binding partners or a mutant binding partner that has reduced binding capability to ZNF827. Without wishing to be bound by theory, such proteins may act as decoys to the endogenous ZNF827 or its binding proteins, saturating the available binding sites on the endogenous proteins and thereby inhibiting their function.

### Methods of Sensitizing

As used herein, the term “sensitizing” shall be taken to mean that a cancer cell is made more susceptible to the effects of an anti-cancer agent relative to a cancer cell in which ZNF827 has not been inhibited. Thus, the methods of sensitizing disclosed herein may comprise enhancing a cancer cell's response to an anti-cancer agent relative to a cancer cell in which ZNF827 has not been inhibited.

The cell may be sensitized in any measurable amount. Sensitization may be complete or may be partial. Thus, the methods disclosed herein may comprise at least partial sensitization of cancer cells. For example, cell sensitization may increase the cell's response to the anti-cancer agent by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% following inhibition of ZNF827.

### Cancer

As used herein, the term “cancer” refers to a disease characterized by the rapid and uncontrolled growth of cells. Cancer cells can spread locally or through the bloodstream and lymphatic system to other parts of the body. Examples of various cancers include, but are not limited to, thoracic cancer, including non-small cell lung cancer and small cell lung cancer, thymoma, thymic carcinoma, thyroid cancer and mesothelioma; head and neck cancer including of the oropharynx, nasopharynx andhypopharynx; melanoma including cutaneous and uveal; skin cancer including basal cell carcinoma, merkel cell carcinoma and squamous cell carcinoma; neurological cancer including glioma, astrocytoma, oligodendroglioma and rare brain tumours; germ cell cancers of any primary site; sarcoma including all sub-types of soft tissue and bone; hepatobiliary cancer including liver, cholangiocarcinoma and gall bladder cancer; upper gastrointestinal cancers including oesophageal, gastric, pancreas and small bowel; lower gastrointestinal cancers including colon, rectal and anal; breast cancer; CNS cancer; gynaecological cancer including ovarian, primary peritoneal, endometrial and vulval; genitourinary cancer including testicular, penile, prostate, bladder and kidney; neuroendocrine and adrenal cancers including carcinoid; cancer of unknown primary; lymphoma including Hodgkin and non-Hodgkin lymphomas, T-cell and B-cell lymphomas of all sub-types; leukaemia including lymphoid and myeloid leukaemia of all sub-types and plasma cell neoplasms including multiple myeloma.

In order to achieve unlimited proliferation cancer cells must maintain their telomeres. The majority of cancers (85-90%) achieve this by reactivating telomerase (telomerase-positive cells). Telomerase is a reverse transcriptase enzyme involved in synthesizing telomeric DNA from an RNA template. The sequence of the catalytic component of telomerase (hTERT) is publicly available. An exemplary sequence is set forth in SEQ ID NO: 15. The remaining 10-15% of tumour cells must stabilize their chromosome ends by alternative mechanisms to avert cessation of growth. These telomerase independent strategies are collectively known as Alternative Lengthening of Telomeres (ALT). Thus, an ALT cell as defined herein may be a cell exhibiting an active ALT mechanism. The ALT mechanism may be any mechanism of telomere stabilization that does not rely on telomerase. The ALT mechanism is not necessarily limited to any one specific mechanism by which ALT may operate in a cell to maintain telomeres. Thus, “ALT mechanism” does not necessarily refer to only one specific biochemical mechanism or pathway by which an ALT mechanism operates. There may be more than one specific pathway by which ALT operates.

In the methods disclosed herein, the cancer may be a telomerase positive cancer or an ALT cancer. In one example, the cancer a telomerase positive cancer. In another example, the cancer is an ALT cancer. In another example, the cancer is not an ALT cancer.

It will be appreciated by those skilled in the art that an ALT cancer cell may be any cell which does not rely on telomerase activity to maintain its telomere length. Conversely, it will be appreciated by those skilled in the art that a telomerase cancer cell may be any cell which does not rely on the ALT mechanism to stabilize its telomere length. Alternatively or in addition, an ALT cancer cell may be considered to be any cell which does not rely on telomerase activity to maintain its telomere stability. Thus, an ALT cancer cell may be considered to be a cell which is not telomerase positive; or a telomerase negative cell.

Alternatively or in addition, an ALT cancer cell may be considered to be a cell in which telomerase expression and/or activity is reduced compared to a telomerase positive cancer cell. Conversely, a telomerase positive cancer cell may be considered to be a cell in which telomerase expression and/or activity is increased compared to an ALT cancer cell. Expression and/or activity of telomerase can be determined by any means known in the art. For example, telomerase expression levels can be determined by quantifying the level of production of telomerase mRNA by any suitable method of mRNA detection (for example but without limitation: quantitative PCR, real time qPCR; next generation sequencing (NGS) methods; nanopore sequencing methods; northern blotting; and others). Alternatively or in addition, telomerase expression levels can be determined by quantifying the level of production of telomerase protein by any suitable protein detection methods. Telomerase protein levels can be detected, for example but without limitation, by western blotting; antibody detection methods (e.g., ELISA; or detection of a label such as a fluorescent label conjugated to an antibody capable of binding specifically to telomerase); and other methods. Alternatively or in addition, telomerase expression and/or activity levels can be determined through performance of a telomerase functional assay, wherein the level of telomerase activity is indicative of the level of expression and/or activity of telomerase in a cell. Telomerase activity may be detected by the Telomerase Repeat Amplification Protocol (TRAP), quantitative TRAP (qTRAP), or by a direct telomerase activity assay, such as that described in Cohen and Reddel, 2008. It will be appreciated that any of the methods of identifying an ALT cancer cell or identifying an ALT cancer or determining whether a subject is suffering from ALT cancer disclosed herein may comprise determining whether a cell is a telomerase positive cell by any of the methods disclosed herein, wherein the cell is identified as not being an ALT cell if that cell is determined to be a telomerase positive cell. It will also be appreciated that any of the methods of identifying a telomerase positive cancer cell or identifying a telomerase positive cancer or determining whether a subject is suffering from a telomerase positive cancer disclosed herein may comprise determining whether a cell is an ALT cell by any of the methods disclosed herein, wherein the cell is identified as not being a telomerase positive cell if that cell is determined to be an ALT cell.

ALT cancer cells may be characterized by elevated levels of DNA damage compared to mortal or telomerase-positive cells, indicative of heightened telomeric replication stress in ALT cells. This heightened telomeric replication stress is attributed to cumulative inadequacies in telomere structural integrity. Frequent or persistent replication fork stalling causes nicks and breaks in the DNA, and it has been hypothesized that the ALT mechanism emanates from stalled replication forks that deteriorate to form double stranded breaks (DSBs), which then provide the substrate for the engagement of homology-directed repair pathways, culminating in break induced telomere synthesis. ALT cancer cells therefore achieve a fine balance between telomere protection and repair activities and telomere damage, and disruption of this balance can be applied as a means of dysregulating the ALT mechanism.

An ALT cancer cell as defined herein may be identified by detection of one or more phenotypic traits of ALT telomere repair or ALT telomeric replication stress, including any one or more of: replication fork stalling above a level that is typical of non-ALT cancer cells (e.g., above a level that is typical of telomerase positive cancer cells or mortal cells); DSB occurrence above a level that is typical of non-ALT cancer cells (e.g., above a level that is typical of telomerase positive cells or mortal cells); and others. It will be appreciated that levels of telomeric replication fork stalling and/or DSBs that are typical of ALT cancer cells can be established through identification and/or measurement of these traits in a sample of ALT cancer cells and in a sample of non-ALT cancer cells (e.g., telomerase positive cancer cells or mortal cells). Suitable threshold levels can then be determined according to the particular methodology used to identify and/or measure these traits, such that a given cancer cell can then be identified as an ALT cancer cell or a non-ALT cancer cell using the same or similar methodology. It will be appreciated that the precise thresholds will vary depending on the samples used to establish those threshold levels and according to the particular analytical methodology used in each instance.

Any of the methods disclosed herein may comprise a step of establishing a reference level of any one or more ALT cancer cell characteristics and/or non-ALT cancer cell characteristics. Alternatively, any of the methods disclosed herein may comprise a step of comparing a measurement of an ALT cancer cell characteristic to a predetermined reference level.

ALT involves recombination-dependent DNA replication (Dunham et al., 2000) and ALT may generate sudden, large increases in telomere length (Murnane et al., 1994), consistent with either a long, linear telomeric template or a rolling mechanism, such as rolling circle amplification (RCA). Cancer cells with ALT activity also undergo rapid decreases in individual telomere lengths (Jiang et al., 2005 and Perrem et al., 2001) leading to a highly heterogeneous telomere length distribution. ALT cancer cells often contain telomeric chromatin within promyelocytic leukemia (PML) nuclear bodies (ALT-associated promyelocytic leukemia nuclear bodies; APBs) (Yeager et al., 1999). Thus, an ALT cancer cell as defined herein may comprise any one or more of these phenotypic traits. For example, an ALT cancer cell may exhibit recombination-dependent DNA replication, and/or may exhibit sudden, large increases in telomere length (e.g., compared to a non-ALT cell such as a telomerase positive cancer cell or a mortal cell), and/or may exhibit heterogeneous telomere length distribution (e.g., compared to a non-ALT cell such as a telomerase positive cancer cell or a mortal cell), and/or may comprise APBs (e.g., a level of APBs that is greater than in a non-ALT cell, such as a telomerase positive cancer cell or a mortal cell). Alternatively, an ALT cancer cell may be identified by the maintenance of telomere length over one or more cell divisions, in the absence of telomerase activity and/or expression. It will be appreciated that telomeres are repetitive DNA sequences present at or near the termini of linear chromosomes. Telomeres in humans typically comprise multiple repeats of the nucleotide sequence 5′-TTAGGG-3′. Thus, the identification of telomere length may comprise determining the number of repeats of this nucleotide sequence.

ALT has been identified in carcinomas arising from tissue including tissue derived from the bladder, cervix, endometrium, esophagus, gallbladder, kidney, liver, lung, brain, bone and connective tissue. ALT has also been found in medulloblastomas, oligodendrogliomas, meningiomas, schwannomas and pediatric glioblastoma multiformes. Accordingly, the ALT cell disclosed herein may be derived from a subject suffering from, suspected of suffering from, or predisposed to, a disease or condition associated with abnormal cellular proliferation. The ALT cell disclosed herein may be a cancer cell. The cancer may be of any physiological origin. In one example, the cancer may be any one of bladder cancer, cervical cancer, endometrial cancer, esophageal cancer, gallbladder cancer, kidney cancer, liver cancer, lung cancer, brain cancer, bone cancer or connective tissue cancer. The ALT cell may be, for example, a sarcoma, a blastoma, a carcinoma, a mesothelioma or an astrocytoma. The sarcoma may be osteosarcoma, malignant fibrous histiocytoma, liposarcoma, synovial sarcoma, fibrosarcoma, chondrosarcoma, rhabdomyosarcoma or leiomyosarcoma. The blastoma may be neuroblastoma. The carcinoma may be a non-small cell lung carcinoma such as lung adenocarcinoma or a breast carcinoma. The mesothelioma may be peritoneal mesothelioma. The astrocytoma may be low-grade astrocytoma, anaplastic astrocytoma, or glioblastoma multiforme. The ALT cell may be a medulloblastoma, oligodendroglioma, meningioma, schwannoma and/or pediatric glioblastoma multiforme. The cancer may be a primary cancer or a metastatic cancer. The metastatic cancer may be of a known or unknown origin.

The cancer cell may be derived from any vertebrate, such as a mammal, and in particular, a human.

As indicated herein, methods of determining whether a cell is an ALT cancer cell or a telomerase positive cancer cell or whether a cancer is an ALT cancer or a telomerase positive cancer are known in the art. For example, the C-circle biomarker is an ALT specific molecule which can be detected using the C-circle assay (Henson et al., 2009 and WO/2011/035375). The entire content of WO/2011/035375 is incorporated herein by reference. Briefly, the C-circle assay comprises extracting DNA from the specimen and subsequently quantifying it. For example, the C-circle can be amplified by rolling circle amplification and the products can be detected.

Any of the methods disclosed in Henson et al., 2009 and/or WO/2011/035375 can be used to identify a cell as an ALT cancer cell in conjunction with the present disclosure. Thus, for example, the methods disclosed herein may comprise identifying a cell as an ALT cancer cell by determining the presence and/or amount of partially double-stranded telomeric DNA circles in a cell, wherein the presence and/or amount of partially double-stranded telomeric DNA circles identifies that cell as an ALT cancer cell. The partially double stranded telomeric DNA circles may comprise a closed circular strand and a linear strand. The circular strand may comprise a C-rich or G rich telomeric sequence. The linear strand may comprise G-rich or C-rich telomeric DNA sequence. The partially double-stranded telomeric circles may comprise repeats of the sequence (CCCTAA)ₙ on the circular strand and/or repeats of the sequence (TTAGGG)ₙ on the linear strand (wherein n is any integer greater than 1). The partially double-stranded telomeric circles may comprise repeats of the sequence (TTAGGG)ₙ on the circular strand and/or repeats of the sequence (CCCTAA)ₙ on the linear strand (wherein n is any integer greater than 1). In one example, the presence and/or amount of partially double-stranded telomeric DNA circles in a cell may be detected using rolling circle amplification.

The circular and/or linear strand may comprise variant telomeric repeat sequences, mutant telomeric repeat sequences and/or non-telomeric sequences.

The partially double-stranded telomeric circles may be detected directly or indirectly. For example, detection may be indirect following rolling circle amplification. The rolling circle amplification may use the circular strand of the partially double-stranded circles as the template. In one example, the detection comprises:
(a) optionally isolating DNA from the cell;
(b) incubating the DNA in the presence of a DNA polymerase and one or more dNTPs under suitable conditions such that polymerase-mediated extension from the incomplete (linear) strand generates concatemers of single-stranded telomeric DNA; and
(c) detecting the concatemers.

The concatemers may be detected by any suitable means such as, for example, hybridisation, sequencing, PCR, molecular beacons, nucleic acid enzymes such as DNA partzymes, or by incorporating suitably labelled dNTPs in incubation step (b). In one example, the concatamers may be detected using a labelled nucleotide probe. The labelled probe may comprise the nucleotide sequence (CCCTAA)ₙ, wherein n is 1 or any integer greater than 1. The label may be any detectable label. For example, the label may be a fluorescent label.

The DNA polymerase may be, for example, φ29 DNA polymerase. Typically, wherein the partially double-stranded telomeric circles comprise repeats of the sequence (CCCTAA)ₙ on the circular strand, the dNTPs consist of dATP, dGTP and dTTP, and optionally dCTP.

The detection of the partially double-stranded telomeric circles may be detection of said circles present within the cell, or alternatively may comprise the detection of said circles in a biological sample, for example derived from a subject. Additionally or alternatively, telomerase activity may be detected by the Telomerase Repeat Amplification Protocol (TRAP), quantitative TRAP (qTRAP), or by a direct telomerase activity assay, such as that described in Cohen and Reddel, 2008 in a biological sample, for example derived from a subject. The biological sample may comprise, for example, blood, urine, sputum, pleural fluid, peritoneal fluid, bronchial and bronchoalveolar lavage fluid, or a tissue section. The sample may be obtained, for example, by fine needle aspiration biopsy. The blood may be whole blood, blood serum or blood plasma.

The rolling circle amplification may be conducted with or without the provision of an exogenous primer. Advantageously, the rolling circle amplification can be conducted without an exogenous primer. Thus, the methods disclosed herein of identifying an ALT cancer cell by performing a C-circle assay may not comprise the use of an exogenous primer.

Other suitable methods to determine ALT activity include, but are not limited to, telomerase quantitative PCR of telomeric DNA and C-circles (Lau et al., 2012); the absence of telomerase activity; the presence of very long and heterogeneous telomeres; the presence of ALT-associated promyelocytic leukemia (PML) bodies (APBs), which contain telomeric DNA and telomere binding proteins (Yeager et al., 1999); elevated telomeric sister chromatid exchange (T-SCE) events and the presence of extrachromosomal telomeric repeat (ECTR) DNA. Tumour samples may also be assessed by combined telomere-specific fluorescence in situ hybridization and immunofluorescence labelling for PML protein (Heaphy et al., 2011).

ALT cancer cells contain a novel form of promyelocytic leukemia (PML) bodies (ALT-associated PML body, APBs) in which PML protein colocalizes with telomeric DNA and the telomere binding proteins hTRF1 and hTRF2. APBs are not found in mortal cells, strains of telomerase-positive cell lines or telomerase-positive tumours (Yeager et al., 1999). Any method known in the art used to detect APBs may be used in conjunction with the present disclosure. For example, APBs may be detected visually. For example, APBs may be visualized by immunohistochemistry (for example, using anti-hTRF1 and/or anti-PML antibodies).

Significant differences in telomere variant repeat content have been found in tumours that use the ALT mechanism and those that do not (Lee et al., 2018). Thus, any method known in the art to determine telomere variant repeat content may be used in conjunction with the present disclosure. For example, whole genome sequencing may be used to determine telomere variant repeat content.

### Methods of Treating Cancer and Subject Selection

The inventors have surprisingly shown for the first time that inhibition of ZNF827 is selectively toxic to cancer cells and that inhibiting ZNF827 in conjunction with an anti-cancer agent inhibits cell proliferation and triggers a surprisingly enhanced apoptotic response. Based on this finding, the inventors have developed and provide herein (i) methods of inhibiting cancer cell viability and/or growth, (ii) methods of treating cancer, (iii) methods of sensitizing a cancer cell to an anti-cancer agent (iv) methods of selecting a subject for treatment or identifying whether a subject suffering from cancer is suitable for treatment with an inhibitor of ZNF827 and/or an anti-cancer agent.

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, reducing size of the cancer, inhibiting tumour growth, inhibiting cancer progression or metastasis, ameliorating or palliating the disease state, and remission or improved prognosis.

As used herein, the term “subject” refers to any animal, for example, a mammalian animal, including, but not limited to humans, non-human primates, livestock (e.g. sheep, horses, cattle, pigs, donkeys), companion animals (e.g. pets such as dogs and cats), laboratory test animals (e.g. mice, rabbits, rats, guinea pigs), performance animals (e.g. racehorses, camels, greyhounds) or captive wild animals. In one embodiment, the “subject” is a human. Typically, the terms “subject” and “patient” are used interchangeably, particularly in reference to a human subject. The subject may be receiving simultaneous, sequential or separate administration of an anti-cancer agent. Thus, the subject may be one who has been prescribed or recommended for a course of treatment comprising both an anti-cancer agent and a ZNF827 inhibitor. The order of administration of the anti-cancer agent and the ZNF827 inhibitor can be varied. The timing of administration of the anti-cancer agent and the ZNF827 inhibitor can vary, provided that the effect of either inhibitor remains present in the subject so as to enhance the effect of the other inhibitor. The subject may be a subject suffering from, suspected of suffering from, or predisposed to, cancer. The cancer may be any cancer disclosed herein.

In one example, the present disclosure provides a method of treating cancer, comprising inhibiting ZNF827 in a subject in need thereof, wherein the subject is receiving simultaneous treatment with an anti-cancer agent. In another example, the present disclosure provides a method of treating cancer, comprising inhibiting ZNF827 in a subject in need thereof, wherein the subject is receiving separate treatment with an anti-cancer agent. In another example, the present disclosure provides a method of treating cancer, comprising inhibiting ZNF827 in a subject in need thereof, wherein the subject is receiving sequential treatment with an anti-cancer agent.

Alternatively or in addition, the methods of the present disclosure comprise inhibiting cancer cell viability and/or growth.

In one example, the present disclosure provides a method of treating cancer comprising inhibiting ZNF827, wherein the subject is not receiving simultaneous, sequential or separate administration of an anti-cancer agent. Thus, the methods disclosed herein may comprise inhibiting ZNF827 as the sole therapeutic modality, or the sole anti-cancer therapeutic modality in cancer other than ALT cancer.

Any anti-cancer agent approved for the treatment of cancer is suitable for optional use in combination with inhibiting ZNF827 as disclosed herein. The DNA damaging agent may be an alkylating agent, an antimetabolite, an anti-tumour antibiotic, a topoisomerase inhibitor, a mitotic inhibitor, a corticosteroid, a PARP inhibitor or any other chemotherapeutic agent.

The alkylating agent may be any one or more of Altretamine, Busulfan, Carboplatin, Carmustine, Chlorambucil, Cisplatin, Cyclophosphamide, Dacarbazine, Lomustine, Melphalan, Oxaliplatin, Temozolomide Thiotepa, or any other alkylating agent.

The antimetabolite may be any one or more of 5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate, Pemetrexed (Alimta®) or any other antimetabolite.

The anti-tumour antibiotic may be any one or more of Daunorubicin, Doxorubicin (Adriamycin®), Epirubicin, Idarubicin, Actinomycin-D, Bleomycin, Mitomycin-C, Mitoxantrone or any other anti-tumour antibiotic.

The topoisomerase inhibitor may be any one or more of Topotecan, Irinotecan (CPT-11), Etoposide (VP-16), Teniposide, Mitoxantrone or any other topoisomerase inhibitor

The mitotic inhibitor may be any one or more of Docetaxel, Estramustine, Ixabepilone Paclitaxel, Vinblastine, Vincristine, Vinorelbine or any other mitotic inhibitor.

The corticosteroid may be any one or more of Prednisone, Methylprednisolone (Solumedrol®), Dexamethasone (Decadron®) or any other corticosteroid.

The PARP inhibitor may be any one or more of olaparib, rucaparib, niraparib or any other PARP inhibitor.

Thus, the anti-cancer agent may be a DNA-damaging agent. The DNA-damaging agent may be irradiation (or ionizing radiation).

Particular examples of suitable anti-cancer agents include, but are not limited to, topoisomerase I inhibitors, (topotecan, irinotecan, camptothecin), topoisomerase II inhibitors (etoposide, teniposide, doxorubicin), PARP inhibitors (olaparib, rucaparib, niraparib), AKT inhibitor VIII, Axitinib, AZ628, Bexarotene, CI-1040, FMK, FR-180204, GW441756, I-BET-762, Imatinib, KIN001-236, KIN001-244, KIN001-260, Nilotinib, NPK76-II-72-1, NVP-BHG712, OSI-930, PD0325901, Phenformin, SNX-2112, Sunitib, T0901317, TAK-715, Tamoxifen, THZ-2-102-1, THZ-2-49, TL-1-85, TL-2-105, Tretinoin, VNLG-124, Vorinostat and VX-702. In one example, the anti-cancer agent is topotecan.

In one example, the present disclosure provides a method of selecting a subject for treatment with an inhibitor of ZNF827, the method comprising determining the level of expression and/or activity of ZNF827 in the subject wherein if the level of expression and/or activity of ZNF827 in the subject is normal, the subject is selected for treatment with the inhibitor of ZNF827.

In one example, the present disclosure provides a method of selecting a subject for treatment with an anti-cancer agent, the method comprising determining the level of expression and/or activity of ZNF827 in the subject, wherein if the level of expression and/or activity of ZNF827 in the subject is low, the subject is selected for treatment with the anti-cancer agent.

In one example, the present disclosure provides a method of predicting the response of a subject to an anti-cancer agent, the method comprising determining the level of expression and/or activity of ZNF827 in the subject, wherein a low level of expression and/or activity of ZNF827 in the subject is indicative that the subject's response to the anti-cancer agent is likely improved relative to if the subject had a normal level of expression of ZNF827.

In one example, the present disclosure provides a method of identifying whether a subject suffering from cancer is suitable for treatment with an inhibitor of ZNF827, the method comprising determining the level of expression and/or activity of ZNF827 in the subject, wherein if the level of expression and/or activity of ZNF827 in the subject is normal, the subject is identified as being suitable for treatment with the inhibitor of ZNF827.

In one example, the present disclosure provides a method of identifying whether a subject suffering from cancer is suitable for treatment with an anti-cancer agent, the method comprising determining the level of expression and/or activity of ZNF827 in the subject, wherein if the level of expression and/or activity of ZNF827 in the subject is low, the subject is identified as being suitable for treatment with the anti-cancer agent.

It will be understood by the person skilled in the art that one or more mutations in a subject's ZNF827 nucleotide sequence may affect its expression and/or activity. Thus, the methods disclosed herein may comprise determining the sequence of a subject's ZNF827 nucleotide sequence and comparing it to a reference ZNF827 sequence. The presence of one or more genetic alterations relative to a reference ZNF827 sequence may indicate that the subject has, or is likely to have a reduced level of expression and/or activity. The one or more genetic alterations may include one or more mutations, deletions, insertions, inversions, translocations, epigenetic modifications (for example, but not limited to methylation). Thus, the step of determining the level of expression and/or activity of ZNF827 in a subject in the methods disclosed herein may comprise determining the nucleotide sequence encoding ZNF827 in the subject. Alternatively or in addition, the methods disclosed herein may comprise determining the sequence of a subject's ZNF827 amino acid sequence and comparing it to a reference ZNF827 sequence.

The activity and/or expression of ZNF827 may be measured through any means known in the art, for example through qRT-PCR. Alternative methods including Western blotting, mass spectrometry, immunoprecipitation and others, may also be used. The activity and/or expression of ZNF827 may be measured in a biological sample taken from the subject. The biological sample may comprise one or more cells derived from the subject. Thus, the sample may comprise a tissue sample. Alternatively or in addition, the sample may comprise a bodily fluid comprising one or more cells derived from the subject. Thus, the biological sample may comprise, for example, blood, urine, sputum, pleural fluid, peritoneal fluid, bronchial and bronchoalveolar lavage fluid, or a tissue section. The sample may comprise tissue in which a primary tumour is present or from which a primary tumour is derived. The sample may be obtained, for example, by fine needle aspiration biopsy. The blood may be whole blood, blood serum or blood plasma. Any of the methods disclosed herein may comprise a step of taking a biological sample from a subject and determining the level of activity and/or expression of ZNF827 in the sample.

It will be understood by the person skilled in the art that assays which are used to determine whether a cancer cell is an ALT cell or a cancer is an ALT cancer may also be used to determine whether a subject suffering from ALT cancer is responding to treatment with an inhibitor of ZNF827. Alternatively or in addition, it will be understood by the person skilled in the art that assays which are used to determine to whether a cancer cell is a telomerase positive cell or a cancer is a telomerase positive cancer may be used to determine whether a subject suffering from telomerase positive cancer is responding to treatment with an inhibitor of ZNF827.

The methods of the present disclosure may further comprise the step of determining the nucleotide sequence of ZNF827, determining the level of ZNF827 expression and/or activity or the level of ZN827 protein in the subject prior to treating the cancer. Methods of determining ZNF827 nucleotide sequence, ZNF827 protein expression, ZNF827 protein levels and measuring ZNF827 activity are well-known in the art.

Any of the methods disclosed herein may comprise a step of establishing a reference level of ZNF827 expression and/or activity. Alternatively, any of the methods disclosed herein may comprise a step of comparing a measurement of ZNF827 expression and/or activity to a predetermined reference level. Suitable threshold levels can then be determined according to the particular methodology used to identify and/or measure ZNF827 expression and/or activity. It will be appreciated that the precise thresholds will vary depending on the samples used to establish those threshold levels and according to the particular analytical methodology used in each instance. Thus, a “low” level of ZNF827 expression and/or activity is a level of ZNF827 expression and/or activity that is decreased relative to the reference level of ZNF827 expression and/or activity. Conversely, a “normal” level of ZNF827 expression and/or activity is a level of ZNF827 expression and/or activity that is similar to, equal to, or greater than the reference level of ZNF827 expression and/or activity. The “normal” level of ZNF827 expression and/or activity or the reference level of ZNF827 expression and/or activity can be determined by selecting any suitable population of cells from which to derive the level of ZNF827 expression and/or activity. That population of cells may be taken from any tissue in a subject. For example, the population of cells may be taken from a biological sample as described herein. Thus, for example, the population of cells may be taken from a biological sample comprising tissue in which a primary tumour is present or from which a primary tumour is derived.

In one embodiment, a “low” level of ZNF827 expression and/or activity may be defined relative to the level of ZNF827 expression and/or activity in a population of cells. Thus, the level of ZNF827 expression and/or activity in a population of cells may be ranked in increasing order and a “low” level of ZNF827 expression and/or activity may be defined as being in the lowest 1%, 5%, 10%, 15%, 20%, 25%, 30%, 35% or 40% of the ranked order of ZNF827 expression and/or activity exhibited by that population of cells. For example, the “low” level of ZNF827 expression and/or activity may be defined as being in the lowest 1%, 5%, 10%, 15%, 20% or 25% of the ranked order of ZNF827 expression and/or activity exhibited by that population of cells. Thus, a “normal” level of ZNF827 expression and/or activity may be defined as being in the top 99%, 95%, 90%, 85%, 80%, 75%, 70%, 65% or 60% of the ranked order of ZNF827 expression and/or activity exhibited by that population of cells. For example, the “normal” level of ZNF827 expression and/or activity may be defined as being in the top 99%, 95%, 90%, 85%, 80% or 75% of the ranked order of ZNF827 expression and/or activity exhibited by that population of cells. Again, it will be appreciated that the population of cells may be taken from any tissue in a subject. For example, the population of cells may be taken from a biological sample as described herein. The population of cells may comprise cells taken from a single subject or from multiple subjects. Thus, the population of cells may be derived from a population of individuals. Any suitable number of cells and/or individuals may be sampled in order to provide a statistically meaningful average level of ZNF827 expression and/or activity. In one example, the level of expression of ZNF827 is determined by one or more mRNA quantitation methods. For example, the level of expression may be determined by RT-PCR.

### Pharmaceutical Compositions and Kits

The present disclosure also provides a pharmaceutical composition comprising an inhibitor or ZNF827 and an anti-cancer agent. The pharmaceutical composition may be provided for use in treating cancer.

The present disclosure also provides a pharmaceutical composition comprising an inhibitor of ZNF827 for use in treating cancer, wherein the cancer is not an ALT cancer. In one particular example, the pharmaceutical composition consists essentially of an inhibitor of ZNF827.

The present disclosure also provides the use of an inhibitor of ZNF827 and an anti-cancer agent in the manufacture of a medicament for the treatment of cancer.

The present disclosure also provides the use of an inhibitor of ZNF827 in the manufacture of a medicament for the treatment of cancer. In one particular example, the medicament consists essentially of an inhibitor of ZNF827.

The medicament or the composition may also include excipients or agents such as solvents, diluents, dispersion media, coatings, antibacterial and antifungal agents, isotonic and absorption delaying agents and the like that are physiologically compatible and are not deleterious to the inhibitor as described herein or use thereof. The use of such carriers and agents to prepare compositions of pharmaceutically active substances is well known in the art (see, for example Remington: The Science and Practice of Pharmacy, 21^{st} Edition; Lippincott Williams & Wilkins: Philadelphia, Pa., 2005).

The pharmaceutical composition may be diluted prior to use. Suitable diluents may be selected from, for example: Ringer's solution, Hartmann's solution, dextrose solution, saline solution and sterile water for injection.

The term “composition” as used herein is intended to encompass a product comprising the specified ingredients in the specified amounts, as well as any product which results, directly or indirectly, from combination of the specified ingredients in the specified amounts.

The pharmaceutical compositions include those for oral, rectal, nasal, topical (including buccal and sub-lingual), parenteral administration (including intramuscular, intraperitoneal, sub-cutaneous and intravenous), or in a form suitable for administration by inhalation or insufflation. The inhibitor of ZNF827, together with a conventional adjuvant, carrier or diluent, may be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

The pharmaceutical compositions for the administration of the inhibitors of this disclosure may conveniently be presented in dosage unit form and may be prepared by any of the methods well known in the art of pharmacy.

The pharmaceutical compositions and methods disclosed herein may further comprise other therapeutically active compounds which are usually applied in the treatment of the disclosed disorders or conditions. Selection of the appropriate agents for use in combination therapy may be made by one of ordinary skill in the art, according to conventional pharmaceutical principles. The combination of therapeutic agents may act synergistically to effect the treatment or prevention of the various disorders or conditions disclosed herein. Using this approach, one may be able to achieve therapeutic efficacy with lower dosages of each agent, thus reducing the potential for adverse side effects.

When other therapeutic agents are employed in combination with those disclosed herein, they may be used for example in amounts as noted in the Physician Desk Reference (PDR) or as otherwise determined by one of ordinary skill in the art.

It will be understood, however, that the specific dose level and frequency of dosage for any particular subject may be varied and will depend upon a variety of factors including the activity of the specific compound employed, the metabolic stability and length of action of that compound, the age, body weight, general health, gender, diet, mode and time of administration, rate of excretion, drug combination, the severity of the particular condition, and the subject undergoing therapy.

The pharmaceutical compositions disclosed herein may be delivered to a subject by any suitable means. The pharmaceutical compositions may be targeted specifically to the cancer cells. For example, the pharmaceutical compositions disclosed herein may be provided with one or more delivery vehicles capable of specifically targeting the cancer cells.

The present disclosure also provides a kit comprising an inhibitor of ZNF827 and an anti-cancer agent for treating cancer. The kit may contain instructions for use.

### EXAMPLES

### Example 1. Cell Culture and Cell Lines

All cell lines used in this study are immortal, either tumour derived, or immortalised with the simian virus 40 large T antigen (SV40 LgT) as described in Table.1. Cell lines were cultured in Dulbecco's Modified Eagle Medium (DMEM; D5796, Sigma-Aldrich) supplemented with 10% foetal calf serum (FCS) at 37° C. with 10% CO₂. Cells were grown in monolayers in single use tissue culture flasks (BD Falcon) and subcultured at confluency using standard cell culture passaging protocols. Briefly, following media aspiration and one warm PBS wash, cells were dislodged from the flasks with pre-warmed 0.05% trypsin/EDTA (GIBCO) at 37° C. for 5 minutes. At least four times the volume of warm media was added to deactivate trypsin and homogenously resuspend the cells before transferring appropriate fractions into fresh flasks with sufficient fresh DMEM supplemented with 10% FCS. Cells were typically passaged in a 1:4 to 1:16 split ratio depending on the growth rate of the cell line.

TABLE 1
Cell lines

| | | | Telomere | |
|---|---|---|---|---|
| | Cellular | Method of | Maintenance | |
| Cell Line | Origin | immortalisation | Mechanism | p53 status |
| U-2 OS | Osteosarcoma | Tumour | ALT | Wildtype |
| W138-VA13/ | Fetal lung | SV40 LgT | ALT | SV40 LgT |
| 2RA | fibroblast | | | |
| HT1080 | Fibrosarcoma | Tumour | Telomerase | Wildtype |
| HT1080 6TG | Fibrosarcoma | Tumour | Telomemse | Mutated |

For cryopreservation, cells were pelleted by centrifugation at 200×g for 5 minutes, resuspended in freezing media (90% FCS, 10% DMSO (dimethyl sulfoxide)), and aliquoted into cryogenic vials for storage at −80° C. Vials were transferred to liquid nitrogen for long term storage. To thaw cryopreserved cells, the frozen vial was thawed in a 37° C. water bath, promptly transferred to a 15 mL falcon tube containing 5 mL DMEM supplemented with 10% FCS, then centrifuged at 200×g for 5 minutes. The cell pellet was resuspended in fresh DMEM supplemented with 10% FCS, transferred into a new tissue culture flask, and incubated at 37° C. with 10% CO₂. Media was replaced with fresh DMEM supplemented with 10% FCS to remove non-viable cells after 24 hours.

Topotecan and aphidicolin were used in this study. For experiments where they were used to cause replication stress and DNA damage, cells were treated with topotecan 2 μg/mL for either 1 hour or 24 hours, or aphidicolin 1 μM for 20 hours. For replication fork stalling induction in the PCNA colocalisation experiments, cells were treated with aphidicolin 0.4 μM for 6-8 hours. Topotecan and aphidicolin were dissolved in DMSO. DMSO was used as a control.

### Example 2. ZNF827 Constructs

pCMV6 Myc-DDK-tagged ZNF827 full length (RC221405), and pCMV6-Entry (PS100001) empty vector were purchased from OriGene Technologies. pHTN HaloTag® CMV-neo empty vector was purchased from Promega. pHTN HaloTag® CMV-neo ZNF827 was generated by Sgfl-NotI (New England BioLabs Inc.) restriction enzyme digest of the donor pCMV6 Myc-DDK-tagged ZNF827 plasmid to isolate the ZNF827 insert, followed by gel purification of the ZNF827 insert and ligation into the recipient pHTN HaloTag® CMV-neo empty vector using T4 DNA ligase (New England BioLabs Inc.). Likewise, pCMV6 Myc-DDK-tagged ZNF827 SUMOylation mutant and pHTN HaloTag® CMV-neo ZNF827 SUMOylation mutant constructs were generated by ligation of the mutant insert isolated by Sgfl-NotI digest from a pUC57 ZNF827 SUMOylation mutant construct obtained commercially from Genscript with the respective vectors. The pCMV6 Myc-DDK-tagged ZNF827 ARRK mutant was generated using site directed mutagenesis (Agilent Technologies) (Conomos et al., 2014).

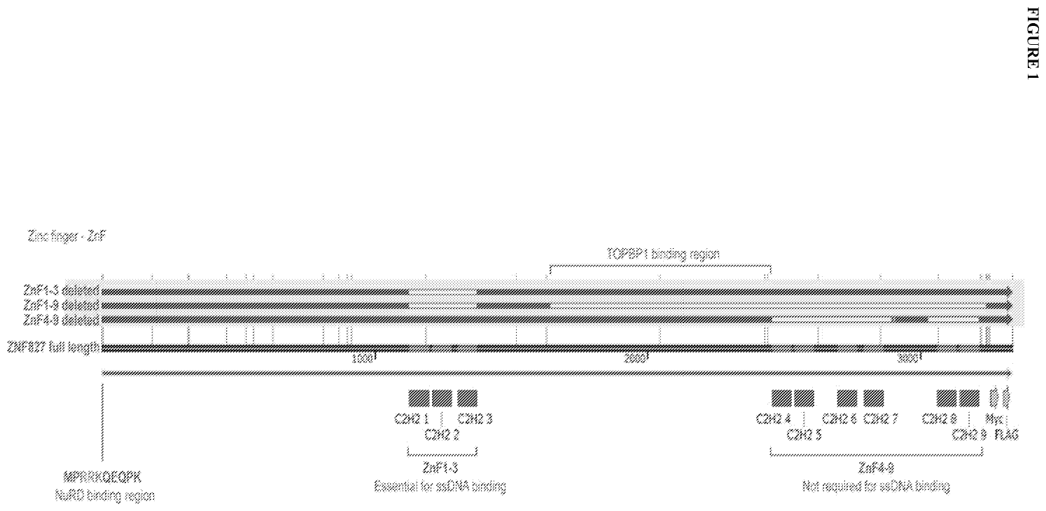

ZNF827 zinc finger mutant plasmid constructs were generated by Genscript, or restriction enzyme digest and ligation. pCMV6 Myc-DDK-tagged ZNF827 ZnF1-3 deleted and pHTN HaloTag® CMV-neo ZNF827 ZnF1-3 deleted mutant constructs were made commercially by Genscript. To generate pCMV6 Myc-DDK-tagged ZNF827 ZnF4-9 deleted mutant construct, ZnF4-9 was excised with BsmI and MluI-HF restriction enzymes (New England BioLabs Inc.) from the full length ZNF827 construct and replaced by a filler fragment, designed with SnapGene and purchased from Integrated DNA Technologies, by ligation using T4 DNA ligase (New England BioLabs Inc.). Similarly, pCMV6 Myc-DDK-tagged ZNF827 ZnF1-9 deleted mutant construct was generated by excision of ZnF 4-9 from the ZNF827 ZnF1-3 deleted construct and ligation with the filler fragment. All three pHTN HaloTag® CMV-neo ZNF827 ZnF mutant constructs were made by isolation of the respective mutant insert from the pCMV6 plasmids and ligation with the pHTN HaloTag® CMV-neo vector, as described above. The ZNF827 constructs are shown in FIG. 1. The nucleotide sequences of the mutants ZNF827 mutant with ZnF1-3 deleted (ZnF1-3 deleted), ZNF827 mutant with ZnF4-9 deleted (ZnF4-9 deleted), ZNF827 mutant with no zinc fingers (ZnF1-9 deleted) and ZNF827 SUMO mutant are set forth in SEQ ID NOs: 16-19, respectively.

All plasmid constructs were verified through Sanger Sequencing. Plasmids were propagated by transformation into One Shot® Stbl3™ chemically competent E. coli, One Shot® TOP10 chemically competent E. coli (ThermoFisher Scientific) or STELLAR chemically competent E. coli (Clontech) according to the manufacturers' instructions. Transformed cells were plated onto agar plates containing the appropriate selection antibiotic, kanamycin 25 μg/mL or ampicillin 100 μg/mL, followed by incubation at 37° C. for 16-18 hours. Aseptically, each single colony was selected with a sterile pipette tip, placed into a mini culture (5 mL LB broth with selection antibiotic), and incubated with vigorous shaking at 37° C. for 8 hours. Turbid mini cultures were transferred into maxi cultures (100 mL LB broth with selection antibiotic), and incubated with vigorous shaking at 37° C. overnight. Plasmid DNA was then extracted from bacteria using the QIAGEN Plasmid Maxi Kit (Qiagen) according to the manufacturer's instructions. All plasmid DNA was stored in 10 mM Tris-Cl, pH 8.0 at −20° C. Glycerol stocks were prepared at a 1:1 ratio of bacteria to glycerol and stored at −80° C.

Overexpression of plasmid constructs was achieved by reverse transfection with FuGENE-6 transfection reagent (Promega). For a 75 cm² transfection, 1 mL reduced serum medium Opti-MEM (Gibco) was combined with 60 μL FuGENE-6 transfection reagent and incubated at room temperature for 5 minutes. 20 μg of plasmid DNA was then added into the mixture (3:1 FuGENE:plasmid ratio), vortexed briefly and incubated at room temperature for 15 minutes. Empty vector plasmid was included as a control. During incubation, cells to be transfected were trypsinised, resuspended in DMEM supplemented with 10% FCS, and counted using a Beckman Coulter cell counter. The prepared transfection mixture was transferred into a 75 cm² flask and swirled gently to cover the flask. 0.8-1×10⁶ cells in 2 mL DMEM supplemented with 10% FCS were directly pipetted onto the transfection mixture in the flask and shaken gently to mix. Additional DMEM supplemented with 10% FCS was added to the flask to a total volume of 15 mL. Cells were incubated at 37° C. for 48 hours prior to harvesting. To harvest, transfected cells were washed once with warm PBS, trypsinised for 5 minutes at 37° C., resuspended in DMEM supplemented with 10% FCS, and counted using a Beckman Coulter cell counter. Known numbers of cells were collected in pellets by centrifugation at 200×g at 4° C., flash frozen in liquid nitrogen and stored at −80° C. for qRT-PCR and Western Blot validation of overexpression, and subsequent experiments.

### Example 3. ZNF827 Knockouts and Knockdowns

### RNA Interference

siRNAs used in this study were purchased from ThermoFisher Scientific and the details are listed in Table 2. Cells were transfected using Lipofectamine RNAiMAX transfection reagent (ThermoFisher Scientific) with an siRNA concentration of 20 μM. For transfection in a 75 cm²flask, 3 mL reduced serum medium Opti-MEM (Gibco) was combined with 15 μL of 20 μM siRNA, mixed gently to cover the surface area of the flask and incubated at room temperature for 5 minutes. 30 μL of Lipofectamine RNAiMAX transfection reagent was then added to the mixture, and mixed and incubated for a further 15 minutes at room temperature. Meanwhile, cells to be transfected were trypsinised, resuspended in DMEM supplemented with 10% FCS, and counted using a Beckman Coulter cell counter. 0.8-1×10⁶ cells in DMEM supplemented with 10% FCS were directly pipetted onto the transfection mixture in the flask, and shaken gently to mix. Additional DMEM supplemented with 10% FCS was added to the flask up to a total volume of 15 mL. Cells were harvested 72 hours post-transfection for knockdown validation and subsequent experiments.

TABLE 2
siRNAs used in this study

| siRNA | Details and Catalogue Number |
|---|---|
| siScrambled | Stealth RNAi ™ Negative Control Med GC |
| | Duplex #2 (12935-112, Invitrogen) |
| siZNF827 | Stealth RNAi ™ siRNA ZNF827HSS135819 |
| | Duplex Oligoribonucleotides (Invitrogen) |
| | 5′-GGG CAG UCU UCU GGC UGA GAA AUC A-3′ |
| | (SEQ ID NO: 12) |
| | 5′-UGA UUU CUC AGC CAG AAG ACU GCC C-3′ |
| | (SEQ ID NO: 13) |

### ZNF827 Knockout Using Clustered Regularly Interspaced Short Palindromic Repeats/Caspase 9 (CRISPR/Cas9)

CRISPR/Cas9 genome editing was performed by the Vector and Genome Engineering Facility (VGEF) at Children's Medical Research Institute to knockout the ZNF827 gene in U-2 OS, HT1080 and HEK 293T cells. 70-90% confluent cells were provided in 6-well plates to the VGEF facility for the CRISPR/Cas9 experiments. The complete CRISPR procedure, including single guide RNA design, plasmid preparation and transfection, clone selection by PCR, expansion and confirmation of knockout clones by pGEM sequencing, was carried out by VGEF. The guide RNA sequence (SEQ ID NO: 20) was GTCTCTGGAGGACCGGATCCAGG (the last three nucleotides are the PAM sequence) which targets exon 2 of the ZNF827 gene. One complete ZNF827 knockout clone was obtained from U-2 OS while no viable clones were obtained from HT1080 and HEK 293T.

### Example 4. ZNF827 is a Novel ssDNA Binding Protein

To investigate whether ZNF827 binds ssDNA, electrophoretic mobility shift assays (EMSAs) were performed with ZNF827 recombinant proteins purified using the HaloTag® protein purification system and radiolabelled double-stranded (ds) and ss telomeric and non-telomeric (pentaprobe) DNA substrates.

Protein purification was performed in HEK 293T cells overexpressing HaloTag® ZNF827 full length (SEQ ID NO: 6), ZNF827 ZnF1-3 deleted (ZnF1-3 del) (SEQ ID NO: 16), ZNF827 ZnF4-9 deleted (ZnF4-9 del) (SEQ ID NO: 17), ZNF827 ZnF1-9 deleted (ZnF1-9 del) (SEQ ID NO: 18), ZNF827 SUMOylation mutant (SUMO Δ) (SEQ ID NO: 19) or empty vector control using the HaloTag® Protein Detection and Purification System (Promega) based on the manufacturer's protocol with minor modifications. Briefly, cell pellets collected in overexpression experiments, were gradually thawed on ice, resuspended homogenously in Mammalian Lysis Buffer (Promega) supplemented with 1× Protease Inhibitor Cocktail (Promega) to a concentration of 2-6×10⁷ cells/mL, and incubated at 4° C. for 1 hour on a rotator. Cell lysates were centrifuged at 10,000×g for 30 minutes at 4° C. to collect the supernatant, which was then diluted 1:3 by adding HaloTag® Protein Purification Buffer. 180 μL/mL lysis buffer of HaloLink™ Resin slurry was equilibrated by five 5-minute washes in 5 mL of HaloTag® Purification Buffer followed by removal of supernatant after centrifugation at 1500×g for 5 minutes between washes. Cell lysates were added to the equilibrated resin, mixed well by inverting the tubes, and incubated on a rotator at 4° C. overnight. The following day, samples were centrifuged at 1500×g for 5 minutes. Supernatants were transferred to another tube as flowthrough fraction for binding efficiency analysis as required. The resin bound with proteins was washed with 5 mL HaloTag® Protein Purification Buffer on a rotator for 3×10 minutes followed by removal of supernatant after centrifugation at 1500×g for 5 minutes between washes. For protein elution, HaloTEV Protease cleavage solution (9 μL of HaloTEV Protease in 291 μL HaloTag® Protein Purification Buffer per 900 μL resin slurry) was added to the settled resin, mixed well and incubated on a rotator at 4° C. overnight. The following day, the supernatant (eluate) was collected by centrifugation at 1500×g for 5 minutes. To remove residual resin, the eluate was transferred to a spin column in a 1.5 mL low bind microcentrifuge tube and collected by centrifugation at 10,000×g for 15 seconds. Eluates were mixed with 10% glycerol to maintain protein stability, aliquoted and stored at −80° C. until use.

Purified proteins were quantified using a BCA assay using the Pierce™ BCA Protein Assay Kit according to the manufacturer's instructions. Equal amounts of purified proteins were incubated in binding buffer (20 mM HEPES-KOH, pH7.9, 100 mM KCl, 0.8 mM ZnCl₂, 0.2 mM EDTA, 5% glycerol, 0.5 mM DTT and 0.5 mM PMSF) with 6.25 μg/mL poly (dI:dC) and 50 ng/μL BSA on ice for 20 minutes. ˜0.12 nM (˜2.5 fmol) γ-³²P labelled DNA oligonucleotide (see Table 3) probes were added to samples and incubated on ice for another 30 minutes. Binding reactions were loaded onto an 8% native acrylamide/bisacrylamide (19:1, Biorad) gel pre-run at 100V in 0.5× TB buffer (89 mM Tris, 89 mM boric acid) for 20 minutes. Gels were electrophoresed at 150 V for 150 minutes at 4° C., dried at 65° C. for 45 minutes, and then exposed to a Phosphor screen overnight. Gel images were obtained by scanning the screens with a Typhoon FLA9500 Imager (GE Life Sciences).

TABLE 3
EMSA probes

| # | Probe | Sequence (5′-3′) | SEQ ID NO: |
|---|---|---|---|
| 1 | ss telomeric G rich oligo | TTA GGG TTA GGG TTA GGG | 21 |
| 2 | ss telomeric C rich oligo | CCC TAA CCC TAA CCC TAA | 22 |
| 4 | ss pentaprobe 3 | CGC TCT ATT CTA CTG TCC TGT GCA TTC | 23 |
| | | AAT CGT TGA GTT CGA TCT AGT CTC GTC | |
| | | TAA CCC TCC CCT GCT CCG CTG GTC TGG | |
| | | CCT CGC CTA TCC TAC CCA T | |
| 5 | ss pentaprobe 9 | ATG GGT AGG ATA GGC GAG GCC AGA CCA | 24 |
| | | GCG GAG CAG GGG AGG GTT AGA CGA GAC | |
| | | TAG ATC GAA CTC AAC GAT TGA ATG CAC | |
| | | AGG ACA GTA GAA TAG AGC G | |
| 6 | ds telomeric oligo | Annealed probe 1 and 2 | |
| 7 | ds pentaprobe 3-9 | Annealed probe 5 and 6 | |

To identify which zinc fingers are required for DNA binding activity, zinc finger mutants with either one or both zinc finger clusters deleted were also generated and purified (FIG. 1). Purified proteins were validated by HaloTag® TMR ligand detection and western blot analysis. Mass spectrometry (MS) analysis also confirmed the specificity of the HaloTag protein purification method. HaloTag® empty vector and the ZNF827 SUMOylation mutant (SUMO Δ) were included in the binding assays as negative and positive controls, respectively, as SUMOylation should not affect DNA binding activity, given that SUMO Δ can associate with telomeres. Double-stranded (ds) telomeric EMSA probes were obtained by radiolabelling annealed complementary 18-mer G-rich and C-rich oligonucleotides digested by Exo VII to remove any residual single-stranded (ss) regions.

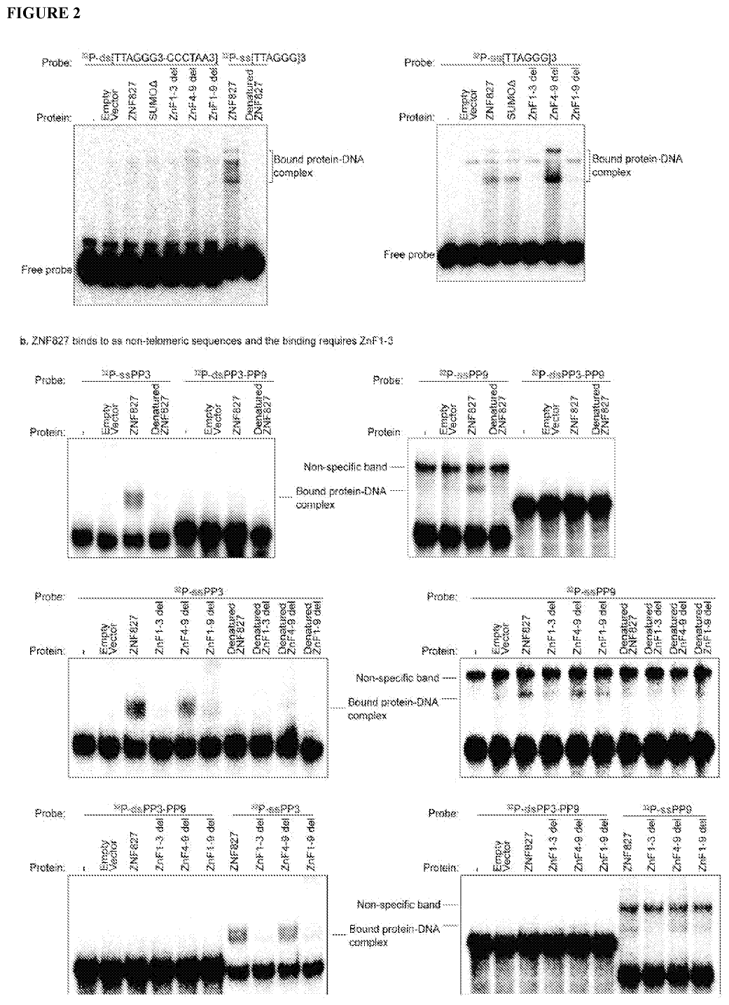

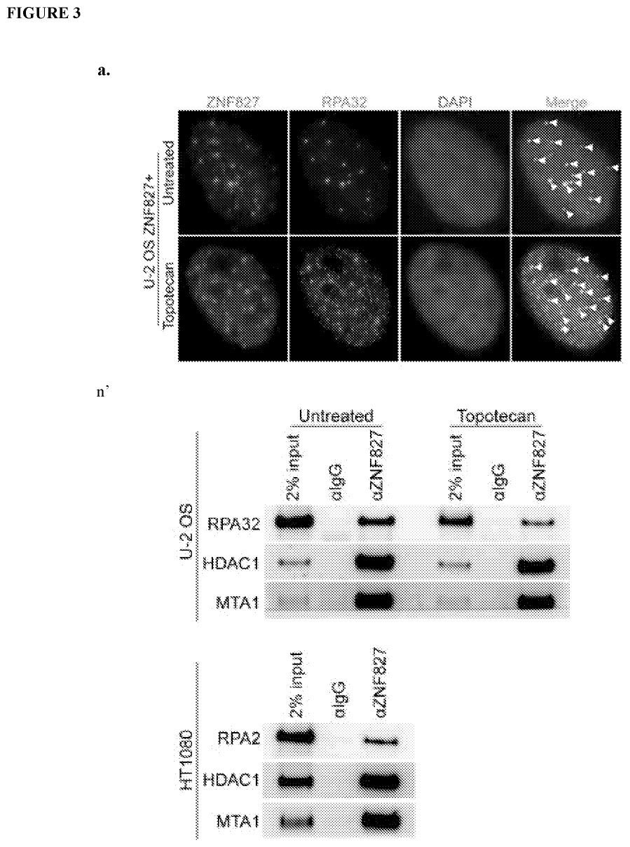

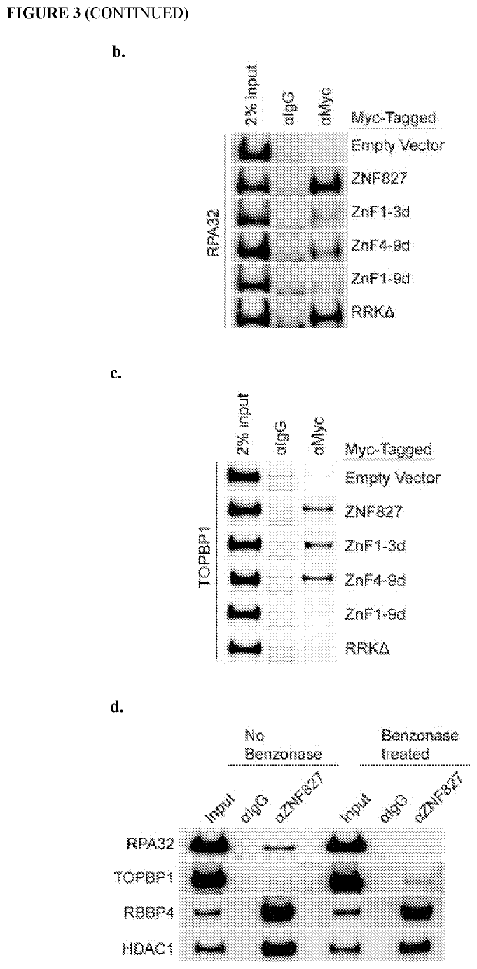

ZNF827 and the mutant proteins were allowed to bind to radiolabelled ds and ss telomeric DNA substrates as shown in FIG. 2a. Unexpectedly, the results demonstrated that neither ZNF827 or any of the mutant proteins bound to ds telomeric probes, but that ZNF827 clearly interacted with the ss G-rich telomeric DNA, as indicated by the shifted bands (FIG. 2a). Further binding experiments between ss telomeric G-rich oligos and ZNF827 (SEQ ID NO: 7) or the mutant proteins (SEQ ID NOs: 16-18), along with empty vector and the SUMO Δ mutant (SEQ ID NO: 19), confirmed binding of ZNF827 to ss G-rich telomeric DNA (FIG. 2a). ZNF827, the SUMO Δ protein and ZnF4-9 del mutant protein exhibited binding activities to the ss G-rich telomeric 18-mer oligos, while the interactions were abolished with the ZnF1-3 del (SEQ ID NO: 16) and ZnF1-9 del (SEQ ID NO: 18) mutant proteins (FIG. 2a). These data demonstrate that zinc fingers 1-3 (ZnF1-3) are required for binding to ss G-rich telomeric DNA. These results indicate that ZNF827 directly interacts with telomeres by preferentially binding to G-rich ss regions that are prevalent in telomeric repeats, and that the binding is dependent on zinc fingers 1-3.

Pentaprobe was employed as a ssDNA substrate to determine whether ZNF827 binds to a wide range of G-rich ssDNA sequence. Pentaprobe is a DNA sequence of minimum length designed to contain all possible 5 base pair sequence motifs for testing the DNA binding activity of proteins (Kwan et al., 2003). This pentaprobe sequence was made into six pairs of overlapping complementary 100-mer ss oligonucleotides. PP3 (SEQ ID NO: 23) and PP9 (SEQ ID NO: 24) were used in this study. ZNF827 as well as the ZnF4-9 del mutant were able to bind to both ssPP3 and ssPP9, but not the ds counterpart (FIG. 2b). The binding was consistently abrogated when zinc fingers 1-3 were deleted in the ZnF1-3 del mutant, corroborating that zinc fingers 1-3 are essential for the binding of ZNF827 to ss DNA (FIG. 2b). Taken together, the data from these experiments demonstrate that ZNF827 binds via zinc fingers 1-3 to diverse ssDNA sequences with no stringent sequence specificity.

### Example 5. ZNF827 Interacts Directly with RPA and TOPBP1

Replication protein A (RPA) is the most well-studied ssDNA binding protein with no sequence specificity, known for its established roles in DNA replication, recombination and repair (Zou et al., 2006). The inventors have demonstrated that ZNF827 is a novel ssDNA binding protein. Considering the similarities between ZNF827 and RPA, the inventors explored whether there is a direct association between them using immunofluorescence (IF) and co-immunoprecipitation (co-IP).

### Indirect Immunofluorescence (IF)

Cells cultured on coverslips were fixed in 2% paraformaldehyde in PBS at room temperature for 10 minutes. Cells fixed on coverslips were rinsed with deionised water, and then permeabilised using KCM buffer (120 mM potassium chloride, 20 mM NaCl, 10 mM Tris, pH 7.5, 0.1% Triton X-100) at room temperature for 10 minutes. Coverslips were blocked with antibody diluent buffer (ABDIL—20 mM Tris-Cl, pH 7.5, 2% BSA, 0.2% fish gelatine, 150 mM NaCl, 0.1% Triton X-100, 0.1% sodium azide) containing 100 μg/mL RNase A at room temperature for one hour. Primary antibodies diluted in ABDIL were added to the coverslips, and incubated at room temperature with gentle rocking for 1 hour or at 4° C. overnight (refer to Table 4 for the list of antibodies). Coverslips were washed in PBST three times for 10 minutes with shaking, and then incubated with appropriate fluorophore conjugated secondary antibodies diluted 1:500 in ABDIL at room temperature with gentle rocking in the dark for 1 hour (refer to Table 5 for the list of secondary antibodies). Following three 10-minute PBST washes with shaking, coverslips were incubated with 50 ng/mL DAPI in PBS for 15 minutes followed by 2×5 minutes washes in PBST and a quick rinse in deionised water. After airdrying, slides were mounted in Prolong Gold Antifade (Invitrogen) and stored at 4° C. until microscope analysis. Colocalisations in this study were defined by at least 50% overlap of two protein immunofluorescence signals. Quantitated analysis was conducted by automation with CellProfiler (Broad Institute).

TABLE 4
Primary antibodies used in this study

| Antibody | Source | Catalog No. | Dilution | Applications | Host |
|---|---|---|---|---|---|
| ZNF827 | Santa Cruz | sc-249818, | 1 in 200 | WB, IF, IP, | Goat |
| | | T20 | (WB/IF); | PLA, ChIP | |
| | | | 1 μg/100 μL | | |
| | | | lysate (IP); 15 | | |
| | | | μL/110 μL | | |
| | | | (ChIP) | | |
| Myc-Tag | Cell Signaling | 2276, 9B11 | 1 in 1000 | WB, IF, ChIP | Mouse |
| | Technologies | | (WB); 1 in | | |
| | | | 4000 (IF)s | | |
| HDAC1 | Cell Signaling | 5356, 10E2 | 1 in 1000 | WB | Mouse |
| | Technologies | | | | |
| MTA1 | Cell Signaling | 5646, | 1 in 1000 | WB | Rabbit |
| | Technologies | D17G10 | | | |
| pCHK1 | Cell Signaling | 2348, 133D3 | 1 in 1000 | WB | Rabbit |
| (S345) | Technologies | | | | |
| CHK1 | Cell Signaling | 2360, 2G1D5 | 1 in 1000 | WB | Mouse |
| | Technologies | | | | |
| pRPA2 (S33) | Bethyl lab | A300-246A | 1 in 2000 | WB | Rabbit |
| RPA2 | Abcam | ab2175 | 1 in 500 | WB, IF, IP | Mouse |
| | | | (WB/IF); 1 | | |
| | | | μg/100 μL | | |
| | | | lysate (IP) | | |
| ATR | Abcam | ab2905 | 1 in 500 | IF, PLA | Rabbit |
| ATR | Cell Signaling | 2790 | 1 in 1000 | WB | Rabbit |
| | Technologies | | | | |
| p21 | Cell Signaling | 2947, 12D1 | 1 in 1000 | WB | Rabbit |
| | Technologies | | | | |
| p53 | Santa Cruz | D01 | 1 in 1000 | WB | Mouse |
| p-γH2AX | Millipore | 05-636, | 1 in 1000 | WB | Mouse |
| (S139) | | JW301 | | | |
| Normal IgG | Cell Signaling | 2729 | 1 μg/100 μL | IP, ChIP | Rabbit |
| | Technologies | | lysate (IP); 5 | | |
| | | | μL/110 μL | | |
| | | | (ChIP) | | |
| Normal IgG | R&D Systems | AB108C | 1 μg/100 μL | IP, ChIP | Goat |
| | | | lysate (IP); 5 | | |
| | | | μL/110 μL | | |
| | | | (ChIP) | | |

TABLE 5
Secondary antibodies used in this study

| | | | | Appli- | |
|---|---|---|---|---|---|
| Antibody | Source | Catalog No. | Dilution | cations | Host |
| HRP anti- | Dako | P0447 | 1:5000 | WB | Polyclonal |
| Mouse IgG | | | | | Goat |
| HRP anti- | Dako | P0448 | 1:5000 | WB | Polyclonal |
| Rabbit IgG | | | | | Goat |
| HRP anti- | Dako | P0449 | 1:5000 | WB | Polyclonal |
| Goat | | | | | Rabbit |
| IgG | | | | | |
| Alexa Fluor | Invitrogen | A-11055 | 1:500 | IF | Donkey anti- |
| 488 | | | | | goat |
| | Invitrogen | A-21202 | 1:500 | IF | Donkey anti- |
| | | | | | mouse |
| | Invitrogen | A-21206 | 1:500 | IF | Donkey anti- |
| | | | | | rabbit |
| Alexa Fluor | Invitrogen | A-11058 | 1:500 | IF | Donkey anti- |
| 594 | | | | | goat |
| | Invitrogen | A-21203 | 1:500 | IF | Donkey anti- |
| | | | | | mouse |
| | Invitrogen | A-21207 | 1:500 | IF | Donkey anti- |
| | | | | | rabbit |
| Alexa Fluor | Invitrogen | A-31571 | 1:500 | IF | Donkey anti- |
| 647 | | | | | mouse |
| | Invitrogen | A-21447 | 1:500 | IF | Donkey anti- |
| | | | | | goat |

### Co-Immunoprecipitation

Cell pellets of 15-20×10⁶ cells, collected in overexpression experiments, were thawed on ice, and resuspended homogenously in 1 mL lysis buffer (20 mM HEPES-KOH pH 7.9, 200 mM NaCl, 2 mM MgCl2, 10% glycerol, 0.1% Triton X-100, 1 mM dithiothreitol (DTT), 1× complete protease inhibitor (CPI), and 1 mM phenylmethylsulphonyl fluoride (PMSF)). Resuspended cells were lysed by incubation with rotation for 1 hour at 4° C. For Benzonase treatment to remove DNA, samples were incubated in the lysis buffer described above supplemented with 25 U Benzonase nuclease (Novagen, Merck Millipore) for 2 hours at 4° C. Lysates were subjected to centrifugation at 13,000 rpm for 40 minutes at 4° C. to remove cell debris. The supernatant was then transferred to a fresh cold low bind 1.5 mL tube for subsequent immunoprecipitation. For immunoprecipitation, protein lysates were incubated with antibody for the protein of interest (10 μg per mL lysate) or IgG control (Refer to Tables 4 and 5 for the list of antibodies used in this study), preincubated with Dynabeads Protein G, as per manufacturer's instructions (Life Technologies), with rotation overnight at 4° C. The following day, beads were washed three times in cold lysis buffer and separated on a magnetic rack. Proteins were then eluted from the beads by resuspension in 50 mM Glycine pH 2.8 and sodium dodecyl sulphate (SDS) sample buffer (0.2 M Tris-Cl, pH 6.8, 28% glycerol, 13.5% β-mercaptoethanol, 6% SDS, 6 mM EGTA, and 0.07% bromophenol blue) for 10 minutes at 70° C. and subjected to Western blot analysis, as described below, to detect various protein interactions.

### Western Blot Analysis

Protein was extracted from 10⁶ cells per sample by vigorous pipetting and incubation at room temperature for 30 minutes in 100 μL of EDTA-free 4× LDS buffer (106 mM Tris-Cl, 141 mM Tris-Base, 40% Glycerol, 2% LDS, 0.075% SERVA Blue G50) supplemented with 2.5% Benzonase® nuclease 25 U/μL (Novagen, Merck Millipore) and 2% beta-mercaptoethanol (Sigma). Following protein extraction, all samples were denatured at 90° C. for 15 minutes before resolving on NuPAGE® Novex® 4-12% Bis-Tris Mini Gels (Invitrogen) with 1×10⁵ cells (10 μL) loaded per lane in NuPAGE® MES SDS running buffer (Invitrogen) at 200 V for 50 minutes. Gels were transferred onto a PVDF membrane (Immobilon-P, Millipore) in transfer buffer (190 mM glycine, 25 mM Tris and 10% methanol) at 100 V for 60 minutes. Membranes were stained with Ponceau-S for 30 minutes, de-stained then blocked for 1 hour at room temperature in 5% skim milk dissolved in PBS with 0.1% Tween-20 (PBST) or TBS with 0.1% Tween-20 (TBST) for phosphorylated proteins. Membranes were cut into appropriate segments based on the size markers, with each segment incubated with antibodies for proteins of interest diluted in 0.5% skim milk in PBST/TBST or 5% BSA overnight at 4° C. (Refer to Table 4 for the list of antibodies). Blots were then washed for 3×5 minutes in PBST/TBST followed by incubation for 1 hour at room temperature with appropriate secondary antibodies (See Table 5) diluted 1:5000 in 0.5% skim milk or 5% BSA in PBST/TBST. Blots were washed for 3×5 minutes in PBST/TBST before incubation with SuperSignal West Pico Chemiluminescent Substrate (ThermoFisher Scientific) for 5 minutes at room temperature. Blots were then exposed to a Luminescent Image Analyser (LAS-4000, Fujifilm).

### Results

Discrete RPA foci were observed in unchallenged U-2 OS cells, consistent with a constant level of DNA damage and homologous recombination (HR) mediated repair events in U-2 OS cells (FIG. 3a). The topoisomerase I inhibitor topotecan greatly induced RPA foci as a result of increased replication associated DNA damage, i.e. stalled or collapsed replication forks. ZNF827 strikingly associated with RPA, in both unchallenged and topotecan-treated U-2 OS cells (FIG. 3a).

Direct interaction between ZNF827 and RPA was further supported by co-immunoprecipitation (co-IP) experiments. RPA was immunoprecipitated with ZNF827 in the U-2 OS ALT cell line and in the HT1080 telomerase-positive cell line, indicative of a direct interaction between the two proteins irrespective of telomeres (FIG. 3a). Co-IP with an RPA32 antibody also pulled down ZNF827, further strengthening the interaction. The ZNF827 and RPA interaction was also detected in topotecan-treated cells at similar levels. Co-IP of ZNF827 and the ZNF827 mutants with Myc antibody demonstrated that deletion of either ZnF1-3 or ZnF4-9 was detrimental to the ZNF827-RPA interaction, with ZnF1-3 deletion exerting a greater effect, while loss of both zinc finger clusters led to complete abolishment of the interaction (FIG. 3b). These results have established that the ZNF827-RPA interaction depends on ZnF1-3 with ssDNA binding capability, and partly depends on ZnF4-9. To delineate whether DNA is required for the ZNF827-RPA interaction, co-IP with Benzonase nuclease to remove DNA was performed and showed that ZNF827-RPA interaction required the presence of DNA (FIG. 3d). HDAC1 and MTA1 are NuRD components known to interact with ZNF827, and were therefore used as positive controls in the co-IP.

All experiments supported a direct interaction between ZNF827 and RPA. A similar direct interaction between ZNF827 and the ATR kinase activator TOPBP1 was identified (FIG. 3c). Interestingly, deletion of either zinc finger cluster had no effect on the interaction, which however, was abolished in the ZnF1-9d mutant. While zinc fingers were not involved in the ZNF827-TOPBP1 interaction, a region between the two zinc finger clusters was identified as being responsible for this interaction (FIG. 1).

### Example 6. ZNF827 Depletion Exacerbates RPA Induction Following Replication-Associated DNA Damage

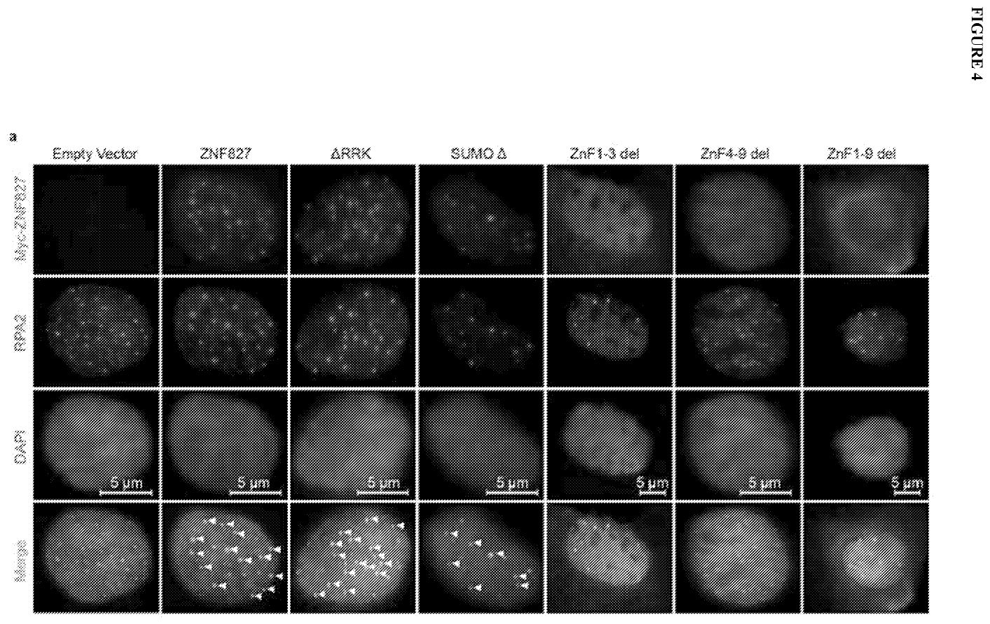

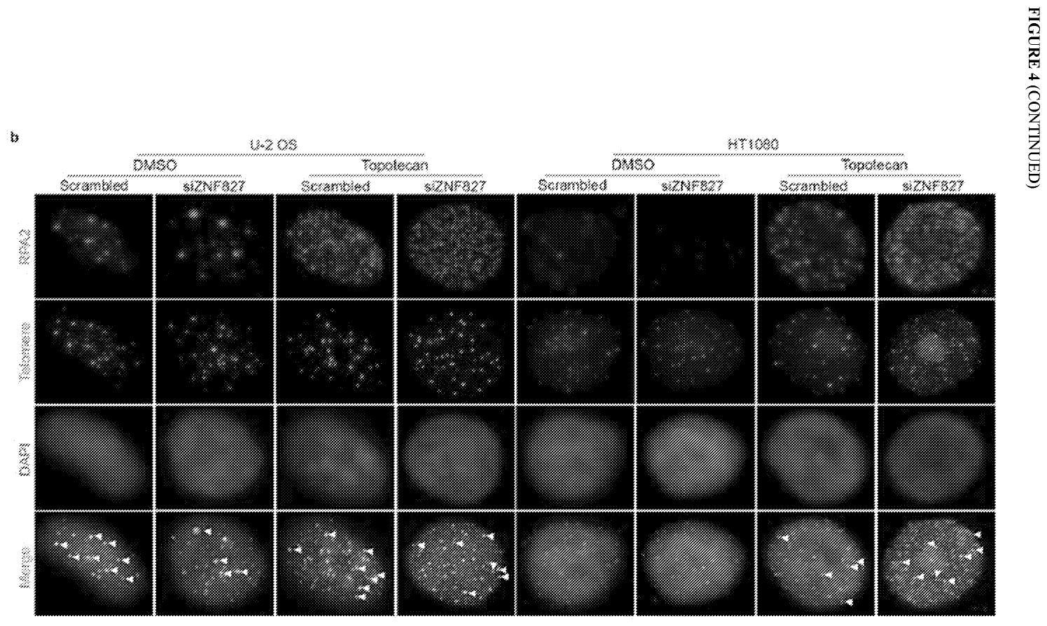

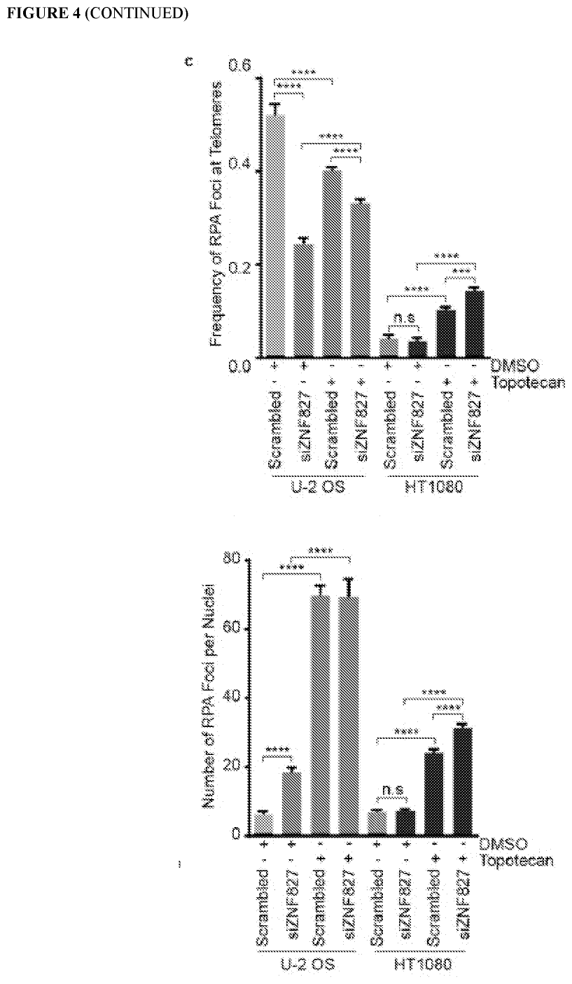

To begin to understand the mechanistic details of the interaction between ZNF827 and RPA, IF was performed (as per Example 5 above) on U-2 OS ZNF827 CRISPR KO cells following exogenous expression of ZNF827 and all five ZNF827 mutants. Neither the RRK or SUMO mutant had an impact on the colocalisation of ZNF827 and RPA, suggesting that their interaction is independent of both the NuRD complex and SUMOylation (FIG. 4a). Amongst the zinc finger mutants, deletion of zinc fingers 1-3 or all zinc fingers consistently showed dispersed cytoplasmic and nuclear localisation with an absence of discrete foci, indicative of an inability for ZNF827 to be retained in the nucleus. Deletion of zinc fingers 4-9 displayed mainly nuclear localisation with widespread smaller indistinct foci (FIG. 4a). Following exogenous expression of the zinc finger mutants, RPA retained its staining pattern (in characteristic nuclear foci), and these foci appeared to be unaffected by the dispersed and aberrant ZNF827 localisation. Large RPA foci were present in the U-2 OS ALT cell line in unchallenged conditions, in contrast to the HT1080 telomerase-positive cell line in which RPA foci only became visible upon topotecan treatment (FIG. 4a and FIG. 4b). There was also a remarkable induction of RPA foci, indicative of robust induction of stalled or collapsed replication forks by topotecan. In U-2 OS, ZNF827 depletion substantially increased the total number of nuclear RPA foci (FIG. 4c). The overall increase in RPA foci suggests that ZNF827 depletion triggers accumulation of ssDNA due to unresolved DNA damage, most likely stalled or collapsed replication forks. In topotecan-treated U-2 OS cells, ZNF827 depletion did not cause an observable increase in overall RPA foci, perhaps due to the massive induction of RPA by topotecan alone (FIG. 4c).

In unchallenged HT1080 cells, depletion of ZNF827 did not change the total number of nuclear RPA foci, possibly because of the very low number of RPA foci observed under unchallenged conditions, which are consistent with the very low spontaneous DNA damage burden in telomerase-positive cells (FIG. 4c). Following DNA damage induction by topotecan, ZNF827 depletion caused a significantly greater increase in overall nuclear RPA foci. These results suggest that ZNF827 plays a role in the repair of DNA damage specifically involving the generation of ssDNA, such as stalled or collapsed replication forks and end resection during HR-mediated repair.

### Example 7. ZNF827 is Involved in the DNA Damage and Repair Pathway

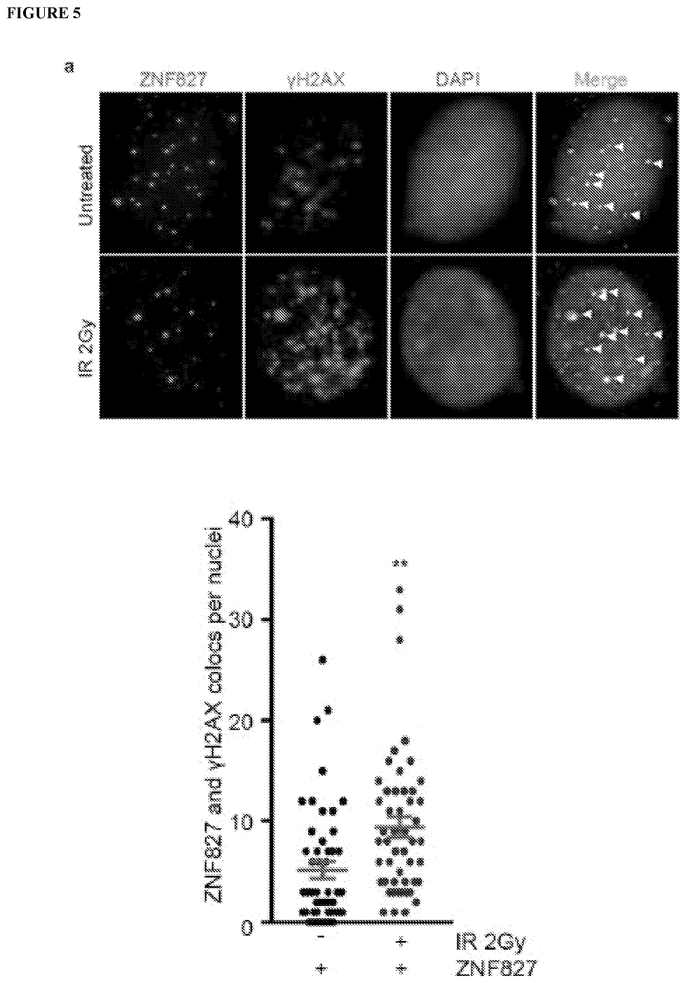

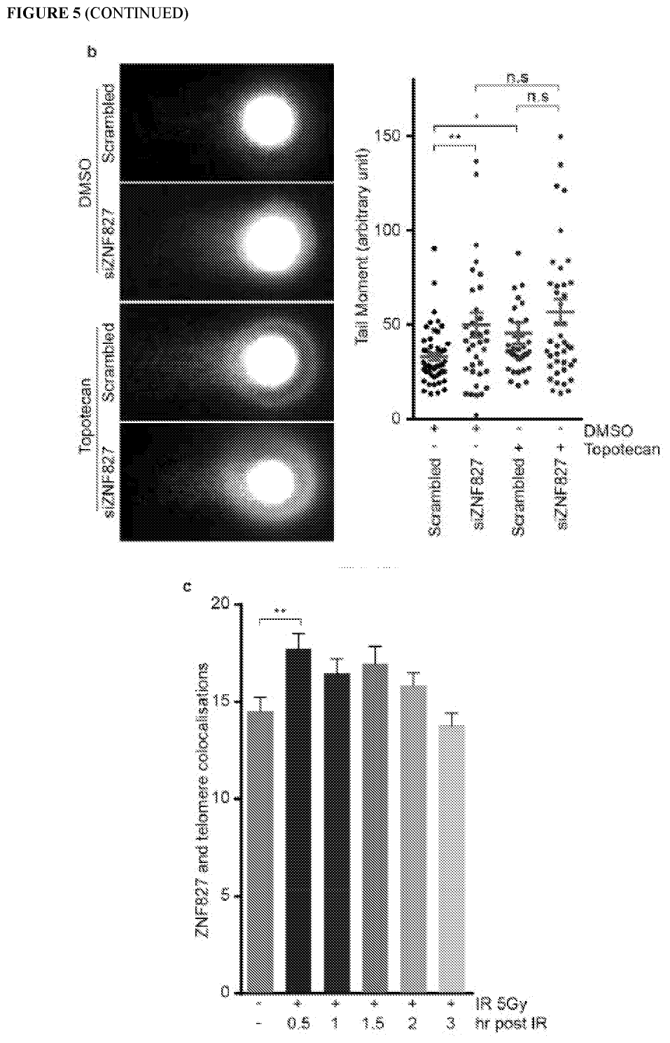

The data in this study so far, in combination with the previous findings that ZNF827 recruits HR proteins BRIT1 and BRCA1 to telomeres and suppresses spontaneous telomeric DNA damage, have implicated ZNF827 in the DNA damage and repair pathway. The functions of ZNF827 with respect to the DNA damage and repair pathway at telomeres and the whole genome were further investigated. DNA DSBs were induced by irradiation (IR) which causes DSBs globally, or by topotecan that results in DSBs from stalled or collapsed replication forks. Colocalisations of ZNF827 and the DNA damage marker γH2AX were clearly observed and increased significantly after IR, supporting the involvement of ZNF827 in the DNA damage response (FIG. 5a). The effect of ZNF827 depletion on DNA damage at a genomic level was explored using the Comet assay.

Comet assays were performed using the CometAssay® kit (Trevigen) according to the manufacturer's protocol with minor modifications. Briefly, cells were counted and resuspended in PBS. 20 μl of cells at 1×10⁵/mL were combined with molten LMAgarose (Trevigen) at 37° C. The cell mixture was pipetted onto the sample area of a prewarmed slide (CometSlide™, Trevigen). Slides were cooled at 4° C. in the dark for 25 minutes, and then immersed in ice-cold lysis solution (CometAssay® kit, Trevigen) at 4° C. overnight. On the following day, slides were removed from lysis solution and gently immersed in ice-cold 1× neutral electrophoresis buffer (100 μM Tris base and 300 μM sodium acetate) for 30 minutes. Slides were then electrophoresed in 1× neutral electrophoresis buffer (100 μM Tris base and 300 μM sodium acetate) for 45 minutes at 1 V/cm. Following electrophoresis, slides were immersed in DNA precipitation solution (1 M ammonium acetate in 95% ethanol) for 30 minutes at room temperature. Slides were then transferred and immersed in 70% ethanol for 30 minutes at room temperature. Slides were then dried for 15 minutes at 37° C., incubated with 1 μM YOYO stain (Invitrogen) for 5 minutes, and Prolong Gold Antifade (Invitrogen) and stored at 4° C. until microscope analysis.

Remarkably, ZNF827 depletion caused DNA damage measured as tail moment to a similar extent as topotecan treatment, while ZNF827 depletion in conjunction with topotecan further increased the amount of DNA damage, suggestive of a role of ZNF827 in suppressing DNA damage (FIG. 5b). Taken together, these data support ZNF827 as a novel player in the DNA damage and repair pathway.

DSBs are predominantly repaired by the two major DNA repair pathways—non-homologous end joining (NHEJ) and homologous recombination (HR). Thus far the inventors' data on ZNF827 including its binding to ssDNA, interaction with RPA, association with replication associated DNA damage all suggest that ZNF827 is involved in HR-mediated DNA repair.

### Example 8. ZNF827 Interacts With ATR

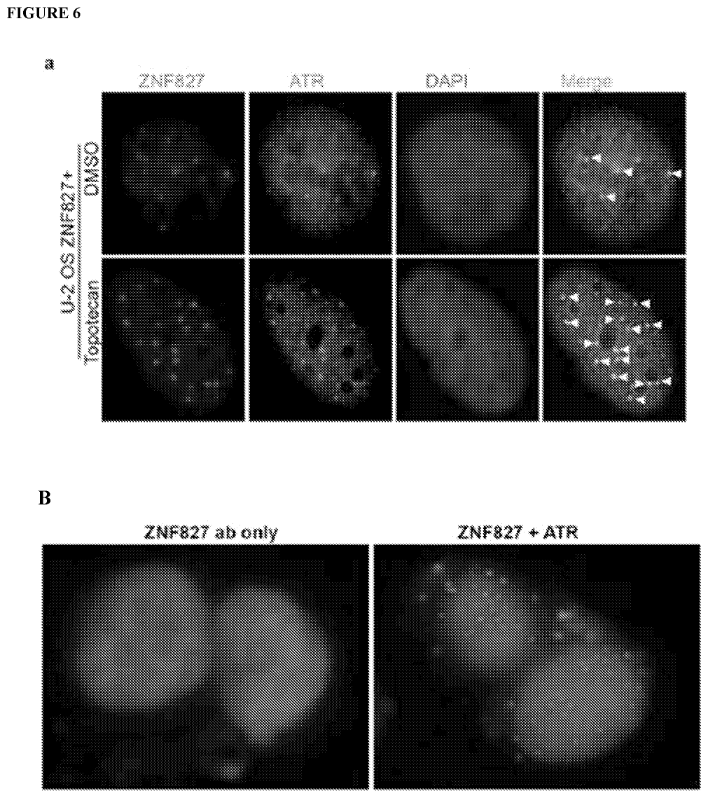

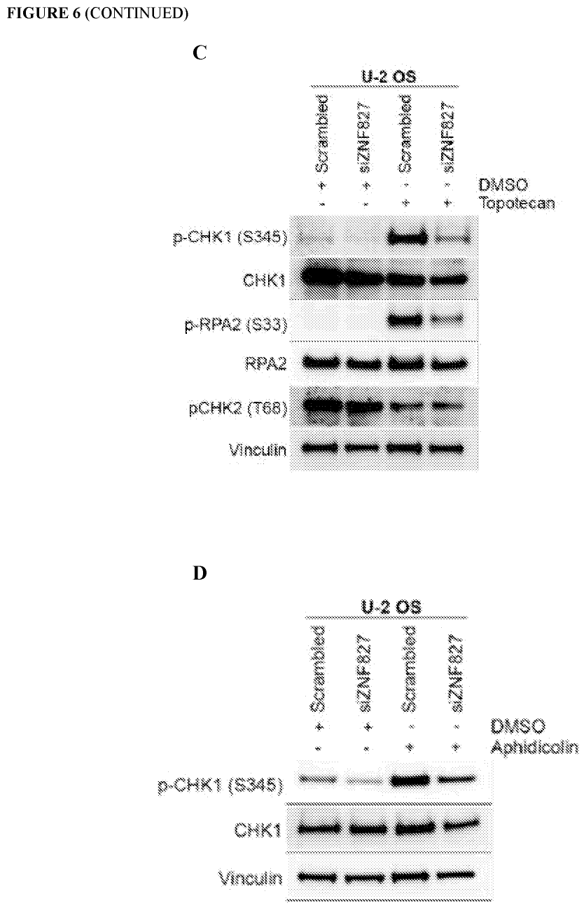

The role of ZNF827 in HR-mediated DNA repair was further investigated. The ATR-mediated DDR signalling pathway plays a critical role in HR-mediated DNA repair, especially at stalled or collapsed replication forks (Cimprich and Cortez, 2008, Flynn and Zou, 2011). The ATR kinase is activated by ssDNA structures coated by RPA, and is the master regulator of cellular responses to replication stress (Flynn and Zou, 2011). Based on the inventors' finding that ZNF827 binds to ssDNA and interacts with RPA, the role of ZNF827 in the ATR DNA damage signalling pathway was explored. ZNF827 visibly colocalised with ATR in unchallenged U-2 OS cells (FIG. 6a). Upon replication associated DNA damage induced by topotecan, the majority of ZNF827 foci colocalised with ATR (FIG. 6a). Details for these experiments may be found in the Indirect Immunofluorescence section in Example 5 and the antibodies used may be found in Tables 4 and 5. This indicates a physical association between ZNF827 and ATR. The interaction between ZNF827 and ATR was further examined using the PLA assay, which permits the identification of weak or transient individual interactions between two proteins in their native form.

### Proximity Ligation Assay (PLA)

Cells at ˜70%-80% confluency were pre-extracted with 0.2% Triton X-100 in PBS for 5 minutes on ice followed by fixation at −20° C. in 70% methanol and 30% ethanol for 30 minutes. Cells were further permeabilised in 1% Triton X-100 in PBS on ice for 10 minutes followed by blocking in antibody dilution buffer (ABDIL—20 mM Tris- Cl, pH 7.5, 2% BSA, 0.2% fish gelatine, 150 mM NaCl, 0.1% Triton X-100 and 0.1% sodium azide) for 1 hour at room temperature. Cells were then incubated with primary antibodies for ZNF827, ataxia-telangiectasia-mutated-and-Rad3-related kinase (ATR) and RPA (Refer to Tables 4 and 5). Following primary antibody incubations PLA was performed using the Duolink® PLA kit as per manufacturer's instructions.

The PLA assay demonstrated a substantial number of PLA signals between ZNF827 and ATR, indicative of a direct interaction (FIG. 6c). Thus, collectively these results suggest that ZNF827 transiently interacts with the ATR kinase.

To investigate the precise function of ZNF827 in the ATR pathway, the effect of ZNF827 depletion on the downstream effectors of ATR was examined. To activate ATR, U-2 OS cells were treated with DNA damage and replication stress inducing agents topotecan 2 μg/mL for 24 hours or aphidicolin 1 μM for 20 hours. As expected, CHK1, the major downstream effector of ATR, became phosphorylated at S345 upon topotecan or aphidicolin treatment, indicative of ATR activation (FIG. 6d and e). RPA, which is also a downstream target of ATR, was phosphorylated at S33 after topotecan treatment (FIG. 6d). Strikingly, both phosphorylation of CHK1 S345 and RPA S33 were markedly suppressed by ZNF827 depletion (FIG. 6d and e). Interestingly even in unchallenged U-2 OS cells, ZNF827 depletion reduced baseline phosphorylation of CHK1 S345. These results suggest that ZNF827 is required for the activation of ATR. Given that ATR plays an essential role in DNA repair involving ssDNA, these data support the findings of the inventors' earlier experiments in which ZNF827 depletion triggered accumulation of ssDNA and unresolved DNA damage (FIG. 4). Phosphorylation of CHK2 was not affected by ZNF827 depletion, further supporting that the role of ZNF827 in DNA damage and repair is independent of ATM (FIG. 6d). Collectively, the data suggest that ZNF827 plays an important role in the activation of the ATR-CHK1 pathway and the repair of DNA damage involving ssDNA, likely stalled or collapsed replication forks.

### Example 9. ZNF827 Localises to Replication Forks and Plays a Role in the Cellular Response to Replication Stress

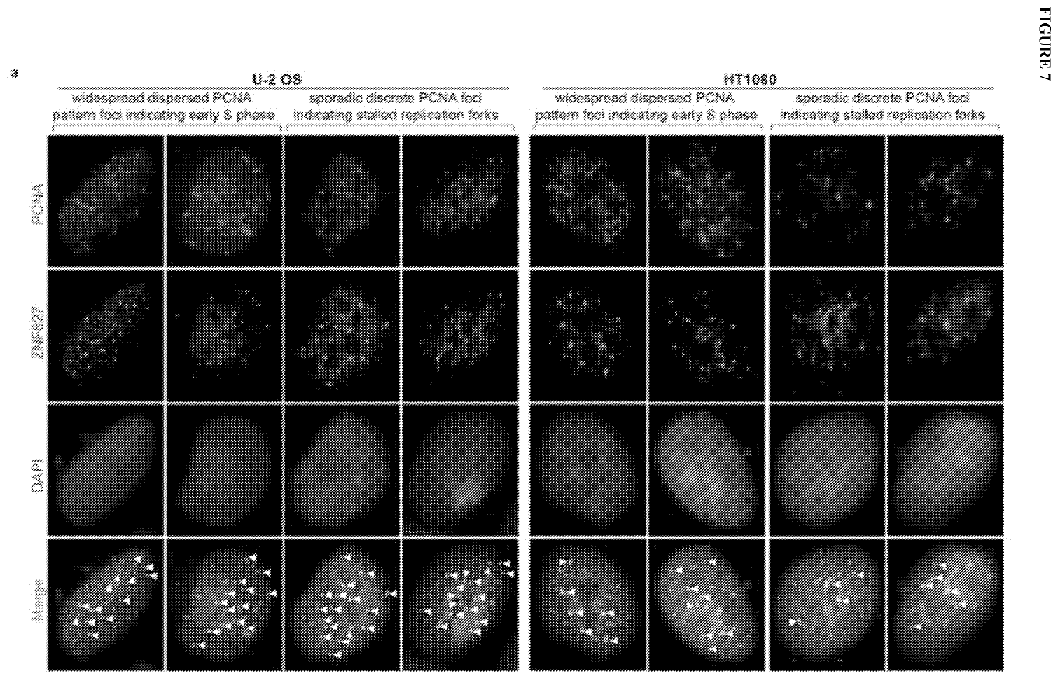

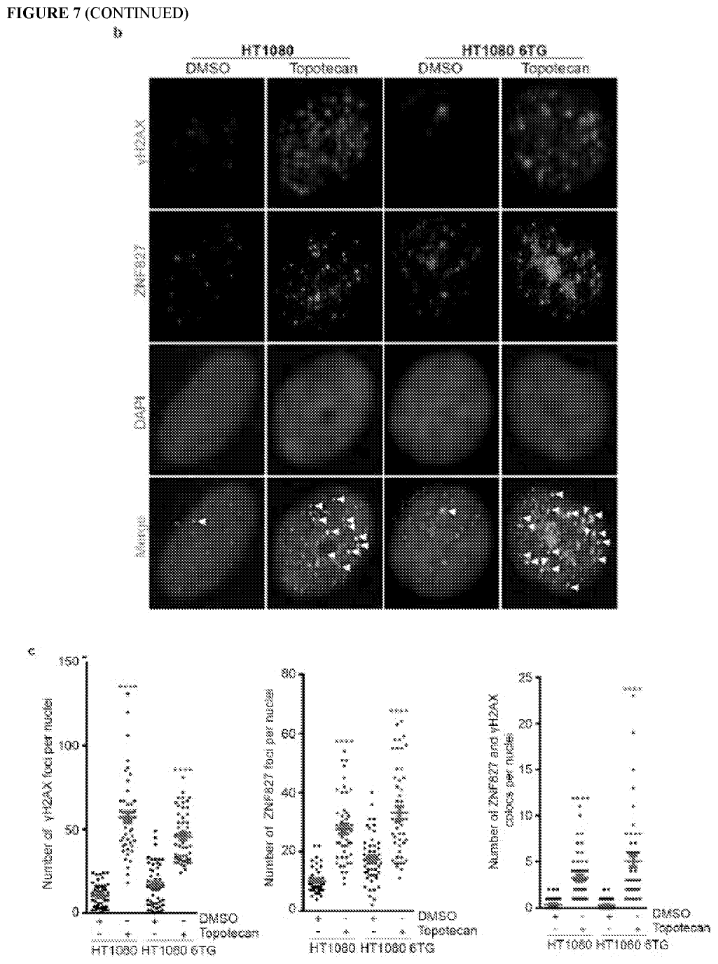

Considering the clear role identified for ZNF827 in the ATR-CHK1 pathway and ATR's fundamental functions in responding to replication stress, the inventors were prompted to investigate the role of ZNF827 in replication stress genomically. ZNF827 colocalised with PCNA, a processivity factor for DNA polymerases that marks replication forks, demonstrating that ZNF827 associates with replication. The nuclear dynamics of PCNA denote the different stages of S phase, with widespread dispersed foci indicating early S phase, and sporadic discrete foci likely to mark stalled replication forks in late S phase (Essers et al., 2005). In FIG. 7a, ZNF827 was shown to colocalise with PCNA in two different nuclear localisation patterns in both U-2 OS and HT1080 cells treated with 0.4 μM aphidicolin for 20 hours to induce stalled replication forks. Particularly in U-2 OS cells, the colocalisations between ZNF827 and PCNA became more pronounced in cells with sporadic discrete PCNA foci, which is indicative of stalled replication forks that form after aphidicolin treatment (FIG. 7a). This implicates the involvement of ZNF827 in processing stalled replication forks.

To further determine whether ZNF827 is present at stalled replication forks, the dynamics of ZNF287 at DNA damage sites specifically resulting from stalled or collapsed replication forks induced by topotecan were examined. Compared to unchallenged HT1080 and HT1080 6TG cells, topotecan efficiently induced γH2AX foci, marking either stalled or collapsed replication forks (FIGS. 7b and c). This coincided with a striking induction of ZNF827, suggesting a rapid response of ZNF827 recruitment to replication-associated DNA damage (FIGS. 7b and c). Additionally, ZNF827 colocalised with topotecan-induced γH2AX foci, indicating that ZNF827 is present at stalled or collapsed replication forks (FIG. 7b and c). Collectively, these data demonstrate that ZNF827 is a novel player in DNA damage and repair, with a specific role in replication-associated DNA damage.

### Example 10. ZNF827 Depletion Induces Replication Defects and Impedes DNA Repair by HR

### Telomere-FISH

To prepare metaphase spreads, sub-confluent cells were treated with 100 ng/mL colcemid (Gibco) in DMEM supplemented with 10% FCS for 4 hours to arrest cells in metaphase prior to harvest. For overexpression experiments, colcemid was added at 44 hours post transfection, and at 68 hours post transfection for siRNA knockdown experiments. As mitotic cells are less adherent, media was collected along with the adherent cells harvested routinely by trypsinisation and centrifugation. Pelleted cells were then resuspended in hypotonic solution (0.2% potassium chloride and 0.2% tri-sodium citrate) for 10 minutes at 37° C. Swollen cells were fixed by gradually adding 1 mL of fresh ice-cold fixative (methanol/acetic acid 3:1), mixed by inversion and incubated on ice for 5 minutes. Cells were then collected by centrifugation at 1200 rpm for 8 minutes. 10 mL ice cold fixative was added to resuspend the cells, followed by 5 minutes incubation on ice, and centrifugation at 1200 rpm for 8 minutes. This fixing step was repeated another two times. Fixed cells were then resuspended in 500-1000 μL of ice-cold fixative, and dropped onto clean, dry microscope slides (HDS Surefrost) with 50-100 μL cell solution per slide. To drop chromosomes, a clean dry slide was held over a 75° C. water bath, and the cell solution was dropped from a pipette onto the slide, which was quickly flipped and held close to the surface of the water bath for 5 seconds. After leaving to dry for 2-3 days in the dark, slides were then blocked with ABDIL containing 100 μg/mL DNase-free RNase A (Sigma-Aldrich) for 30 minutes at 37° C., rinsed in PBS and then fixed in 4% formaldehyde in PBS at room temperature for 10 minutes. Following a quick rinse in deionised water, slides were dehydrated by a graded ethanol series (70% for 3 minutes, 90% for 3 minutes and 100% for 3 minutes), and then air dried. Dehydrated slides were then overlaid with a fluorophore conjugated centromere-telomere PNA probe containing 0.3 μg/mL CENT-Cy3-OO-(AAACTAGACAGAAGCAT (SEQ ID NO: 25)) and 0.3 μg/mL AF488-OO-(CCCTAA)3 (Panagene), denatured for 5 minutes at 80° C. and left to hybridise overnight at room temperature in the dark in a humidified chamber. The following day, slides were washed in PNA wash A (70% formamide and 10 mM Tris-Cl, pH7.5) for 3×5 minutes, and in PNA wash B (50 mM Tris-Cl, pH 7.5, 150 mM NaCl and 0.08% Tween-20) for 3×5 minutes. Slides were then incubated with 50 ng/mL DAPI in PBS for 15 minutes followed by 2×5 minutes washes in PBST and a quick rinse in deionised water. After airdrying, slides were mounted in Prolong Gold Antifade (Invitrogen) and stored at 4° C. until microscope analysis.

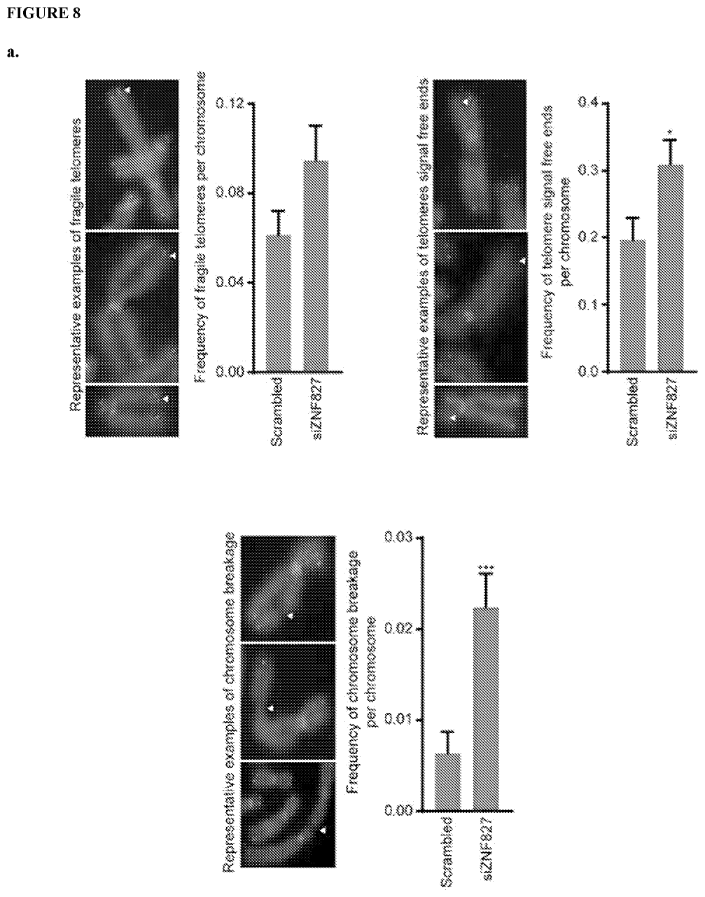

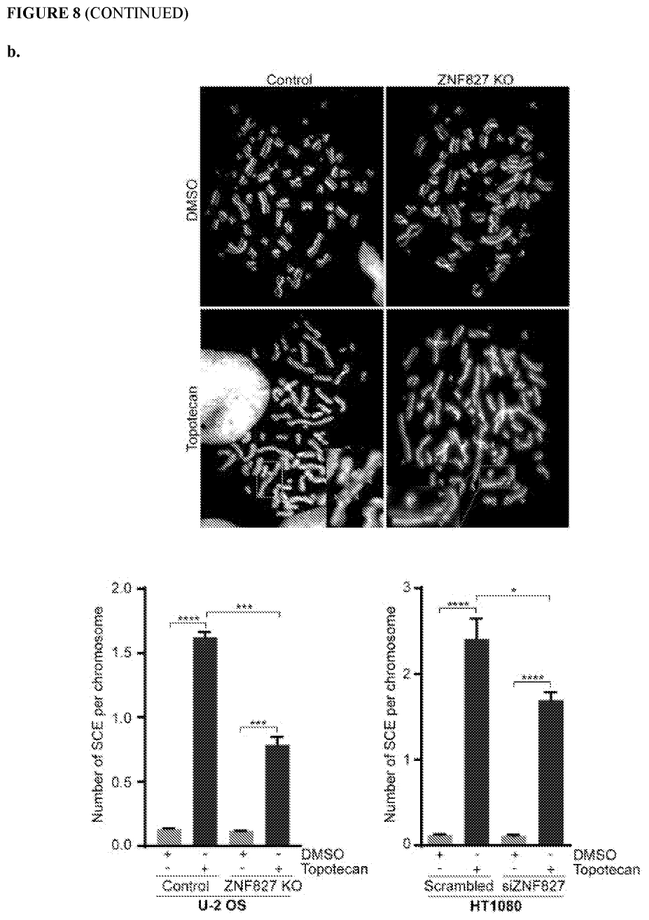

To ascertain the role of ZNF827 in DNA replication dynamics, the effects of ZNF827 depletion on replication were examined by measuring phenotypic markers of replication defects at telomeres as well as genomically. This included quantitation of fragile telomeres, telomere signal free ends, chromosome breakage events and micronuclei. Telomeres are a good model for studying factors that modulate replication stress due to their inherent vulnerability. ALT telomeres, in particular, have elevated levels of replication stress due to structural aberrations. ZNF827 depletion caused clear replication defects at telomeres, demonstrated by an observable increase in the frequency of fragile telomeres, although the difference did not attain statistical significance, and a significant increase in telomeres with signal free ends compared to control (FIG. 8a). Genomically, ZNF827 depletion induced a much greater occurrence of chromosome breakage events (FIG. 8a). Taken together, these results illustrate that ZNF827 exerts a vital function in DNA replication both at telomeres and genome-wide.

Given that ZNF827 is both associated with and induced by replication-associated DNA damage, and that ZNF827 plays an important role in ensuring successful DNA replication, the effects of ZNF827 depletion on the repair of replication-associated DNA damage by HR were explored next. HR is an error free DNA repair pathway predominantly active during replication that uses sister chromatids as templates to ensure faithful DNA repair. The sister chromatid exchange (SCE assay) was used to quantitatively measure HR-mediated repair after induction of DNA damage resulting from collapsed replication forks by topotecan.

For the SCE assay, 20-25% confluent cells were cultured in fresh media supplemented with 7.5 μM 5-bromo-2′-deoxycytidine (BrdU) and 2.5 μM BrdC (BrdU:BrdC 3:1 ratio; Sigma-Aldrich) for 32-48 hours depending on the mitotic index of the cell line. Cell cultures were treated with 100 ng/mL colcemid for the last 4 hours of incubation to accumulate mitotic cells. Cells were harvested by trypsinisation and centrifugation, and then incubated in hypotonic buffer for 10 minutes at 37° C. Swollen cells were fixed by gradually adding 1 mL of fresh ice-cold fixative (methanol/acetic acid 3:1), mixing by inversion and incubating on ice for 5 minutes. Cells were then collected by centrifugation at 1200 rpm for 8 minutes. 10 mL ice cold fixative was added to resuspend the cells followed by 5 minutes incubation on ice and centrifugation at 1200 rpm for 8 minutes. This fixing step was repeated another two times. Fixed cells were then resuspended in 500-1000 μL of ice-cold fixative, and dropped onto clean, dry microscope slides (HDS Surefrost, 50-100 μL cell solution per slide). To drop chromosomes, a clean dry slide was held over a 75° C. water bath, and the cell solution was dropped from a pipette onto the slide, which was quickly flipped and held close to the surface of the water bath for 5 seconds. Slides were left to dry for 2 to 3 days and then treated with 100 μg/ml DNase free RNase A (Sigma) in 2×SSC for 30 minutes at 37° C., rinsed in PBS, and postfixed in 4% formaldehyde in PBS at room temperature for 10 minutes. Following a quick rinse in deionised water, slides were dehydrated in a graded ethanol series (70% for 3 minutes, 90% for 3 minutes, and 100% for 3 minutes) and allowed to airdry. Slides were then stained in 0.5 μg/mL Hoechst 33258 (Sigma-Aldrich) in 2×SSC for 15 minutes at room temperature, rinsed in dH₂O, and air-dried. Slides were then flooded with 200 μL 2×SSC and exposed to long-wave (˜365 nm) UV light (Stratalinker 1800 UV irradiator; Agilent Technologies) for 45 minutes. The BrdU/BrdC-substituted DNA strands were then digested in 10 U/μL Exonuclease III solution (New England Biolabs) in the supplied buffer at 37° C. for 30 minutes. After a quick rinse in deionised water, slides were incubated with 50 ng/mL DAPI in PBS for 15 minutes, washed twice in PBST for 5 minutes, rinsed in deionised water, and airdried. Airdried slides were mounted in Prolong Gold Antifade (Invitrogen) and stored at 4° C. until microscope analysis.

Topotecan caused a drastic induction of SCEs, which was significantly stunted in ZNF827 CRISPR KO U-2 OS cells (FIG. 8b). A similar result was observed in HT1080 cells after ZNF827 knockdown by siRNA. The suppression of SCEs in ZNF827 depleted cells suggests that ZNF827 promotes HR-mediated repair of replication-associated DNA damage, i.e. repair of DSBs from stalled or collapsed replication forks. Given the essential roles of the ATR-CHK1 pathway in safeguarding DNA replication and HR-mediated DNA repair (Syljuasen et al., 2005, Flynn and Zou, 2011, Sorensen et al., 2005), these findings may be explained by inhibition of the ATR-CHK1 pathway caused by ZNF827 depletion, as demonstrated in FIG. 6.

### Example 11. ZNF827 Affects Cell Cycle Progression and its Depletion Causes G1 to Early S Arrest

The findings in this study implicate ZNF827 in mediating the ATR-CHK1 pathway as well as DNA replication, leading the inventors to further explore whether ZNF827 depletion influences cell cycle progression. Using flow cytometry, the effects of ZNF827 depletion on cell cycle progression in unchallenged and topotecan-treated HT1080 cells were examined.

For cell cycle analysis, cells were trypsinised, counted and collected by centrifugation at 1000 rpm for 5 minutes. 1×10⁶ cells were resuspended in 0.5 mL PBS, then fixed in 5 mL of ice-cold 70% ethanol drop by drop whilst being gently vortexed. The cells were incubated on ice for 30 minutes then stored at −20° C. for 2-7 days until propidium iodide staining. Prior to propidium iodide (Sigma) staining, the ethanol fixed cells were centrifuged at 300×g for 6 minutes to remove ethanol, washed once with PBS, and resuspended in 0.5 mL PBS per 1×10⁶ cells. The fixed cells were treated with 0.5 mg of RNase (Sigma) and 25 μg of propidium iodide at 37° C. for 30 minutes, and then allowed to return to room temperature in the dark for 10 minutes. Labelled cells were analysed using the BD FACSCanto Flow Cytometer (BD Biosciences) containing a blue air cooled 488 nm argon laser to excite propidium iodide. Between 10 000-15 000 events were collected at an approximate flow rate of 200 events/second. The forward scatter (FSC, size) and side scatter (SSC, internal granularity) of each cell was recorded. To discriminate and eliminate cell debris and doublets, the pulse area (FL2-A) was plotted against the pulse width (FL2-W). Doublets identified as cells with 4N DNA content and increasing pulse width were eliminated. A histogram displaying the cell counts against the FL2-A was used to calculate the percentage of cells in each cell cycle phase, as well as the percentage of non-viable cells. Data analysis was conducted using BD FACS Diva software (BD Bioscience).

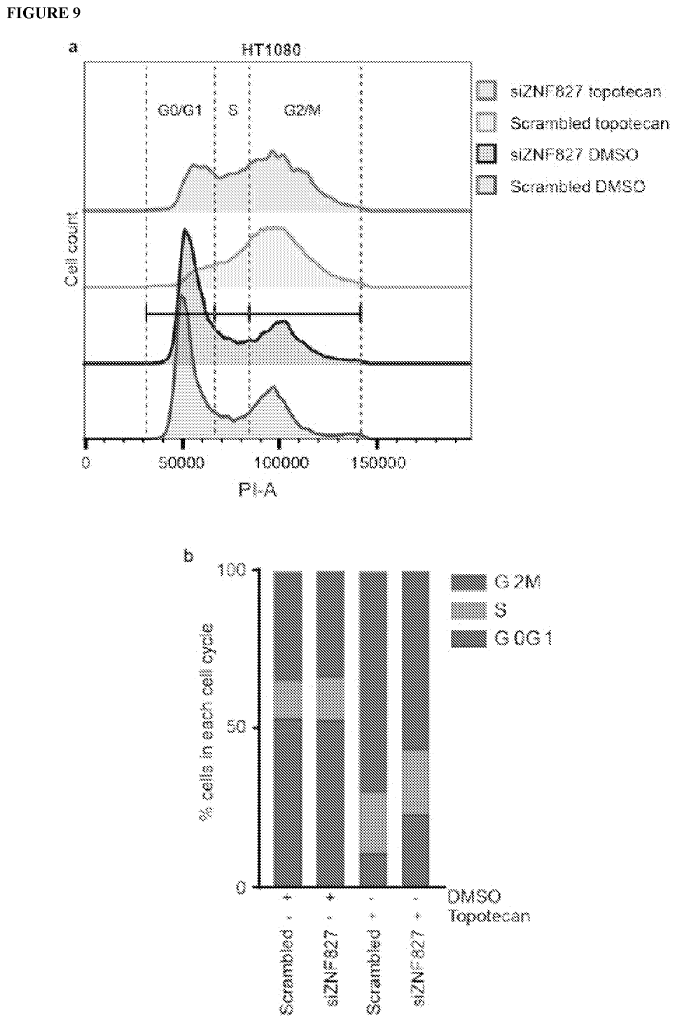

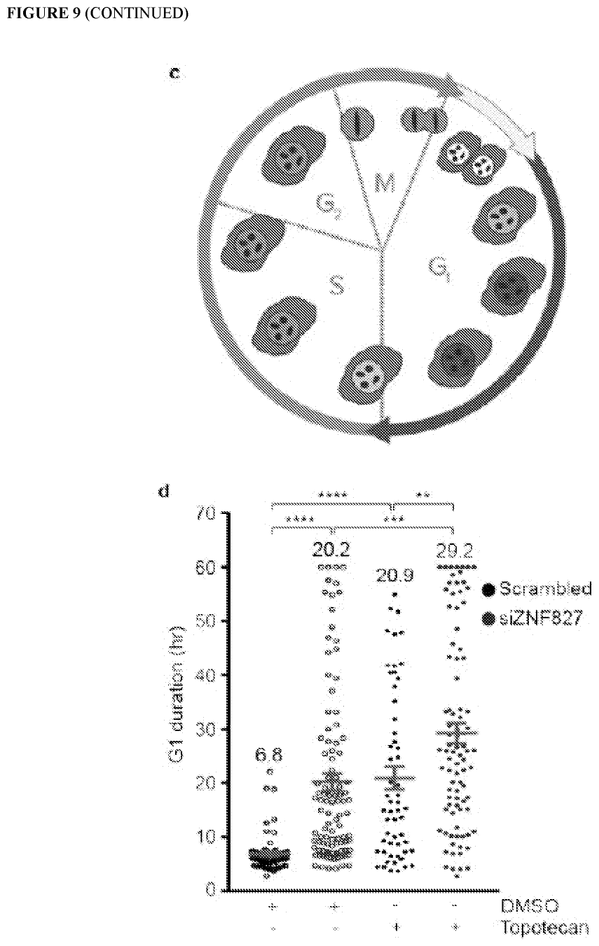

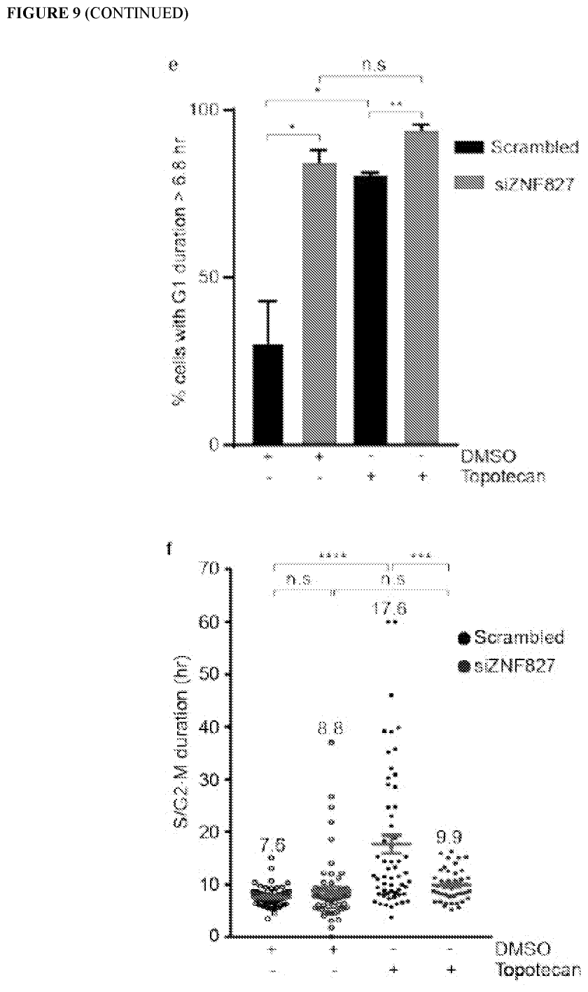

Interestingly in unchallenged cells, depletion of ZNF827 caused a subtle horizontal shift that was most noticeable at the G1/S border. This was reflected in the quantitation as a subtle increase in S phase cells accompanied by a reduction in G2/M cells (FIG. 9 a and b). Topotecan treatment triggered a distinct G2/M arrest, in line with previous studies (Ohneseit et al., 2005, Feeney et al., 2003). ZNF827 depletion in conjunction with topotecan treatment caused a conspicuous G1 arrest while reducing the G2/M arrest (FIGS. 9a and b). These data demonstrate that ZNF827 has an impact on cell cycle progression, most likely by promoting G1/S arrest.

To further characterise the impact of ZNF827 on the cell cycle, live cell imaging was conducted with HT1080 FUCCI cells, to monitor cell progression through the cell cycle in real-time following ZNF827 depletion. The FUCCI (fluorescence ubiquitination cell cycle indicator) cells were stably transfected with fluorescence-labelled cell cycle-regulated proteins, geminin (labelled by GFP) and Cdt1 (labelled by RFP), which are only expressed during specific phases of the cell cycle (FIG. 9c). Their expressions are used to classify cells by cell cycle phase in a live cell setting.

HT1080 FUCCI cells were seeded in glass bottom 12-well plates (MakTek Corporation) the day before imaging at 30% confluency, to ensure that cells were actively cycling for the duration of the experiment. Media was replaced with phenol red free DMEM (Gibco) supplemented with 10% FCS and drug treatment immediately before transferring the plate to the microscope chamber. Imaging was performed on the Cell Observer Widefield Microscope (Zeiss) using a 20× objective at 37° C. with 10% CO2 in a XLmulti S1 full enclosure chamber. Cells were incubated in the XLmulti S1 full enclosure chamber for 2 hours prior to imaging. Three to four positions per well were captured with AxioCam (Zeiss) 506 Mono using ZEN software (Carl Zeiss) with images taken every 6 minutes for 60 hours. More than 15 cells were analysed at each position for each condition to confirm reproducibility.

Noticeably ZNF827 depletion led to a strong G1 arrest comparable to topotecan treatment, while ZNF827 depletion in conjunction with topotecan further exacerbated G1 arrest (FIGS. 9d and e). These results suggest that ZNF827 acts predominantly during the G1/S phase of the cell cycle.

In line with the flow cytometry results, ZNF827 depletion had no impact on G2/M by itself, but resulted in a significant decrease in topotecan induced G2/M arrest, which coincided with the increased G1 arrest (FIG. 9f). These results suggest that ZNF827 acts predominantly during the G1/Sphase of the cell cycle.

### Example 12. Depletion of ZNF827 Suppresses ATR-CHK1 Activation to a Greater Extent than ATR Inhibitors, and Upregulates p21

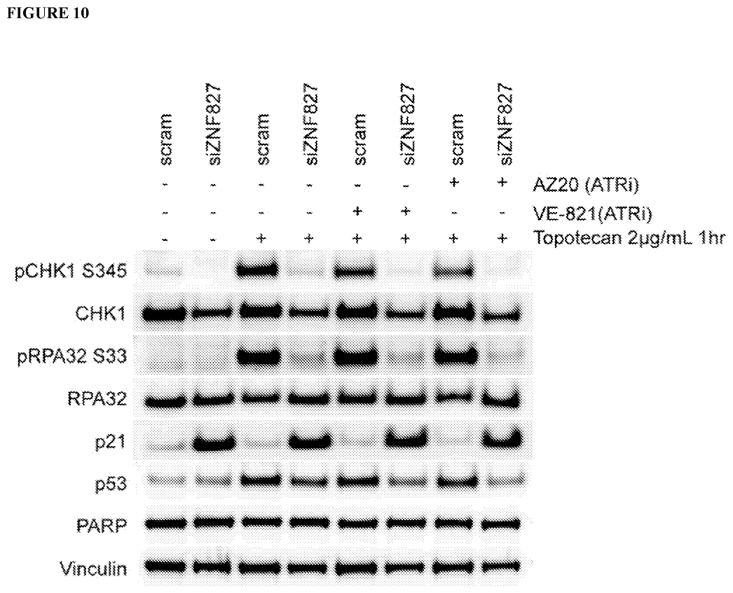

The G1 and early S arrest resulting from ZNF827 depletion coincided with an upregulation of p21 (FIG. 10a), which has been long established to play a crucial role in mediating G1 and S phase cell cycle arrest in response to DNA damage (Niculescu et al., 1998, Ogryzko et al., 1997, Radhakrishnan et al., 2004, Gartel and Radhakrishnan, 2005, Waga et al., 1994). Strikingly, p21 became upregulated even in unchallenged ZNF827-depleted cells to a similar extent as topotecan-treated cells, further reinforcing that ZNF827 depletion induces DNA damage (FIG. 10). Phosphorylation of CHK1 S345 and RPA S33, both of which were downstream targets of the ATR-CHK1 pathway were substantially suppressed by ZNF827 depletion. The drastic abolishment of CHK1 and RPA activation again indicates that ZNF827 depletion suppresses ATR-CHK1 activation. Furthermore, ZNF827 depletion caused a greater reduction in phosphorylation of CHK1 S345 and RPA32 S33 than ATR inhibitors, VE-821 and AZ20.

### Example 13. ZNF827 Inhibition Sensitises Cancer Cells to Anti-Cancer Agents

Using EdU flow cytometry (5-ethynyl-2′-deoxyuridine) is a thymidine analogue which is incorporated into DNA during active DNA synthesis), the effects of ZNF827 depletion on cell cycle progression, especially on DNA replication, were examined.

EdU flow cytometry was performed with the Click-iT EdU flow cytometry kit (ThermoFisher Scientific) according to manufacturer's instructions. Briefly cells were pulsed with EdU for 1 hour at 37° C. and harvested by trypsinisation and centrifugation at 1000 rpm for 5 minutes. Cells were then fixed and labelled with Alex Fluor 647 with the flow cytometry kit following the manufacturer's protocol. For DNA content labelling, the fixed cells were treated with 0.5 mg of RNase (Sigma) and 25 μg of propidium iodide at 37° C. for 30 minutes. Labelled cells were analysed using the BD FACSCanto Flow Cytometer (BD Biosciences) containing lasers to excite Alexa Fluor 647 labelled EdU and propidium iodide. Between 10 000-15 000 events were collected at an approximate flow rate of 200 events/second. Data analysis was conducted using FlowJo 7.

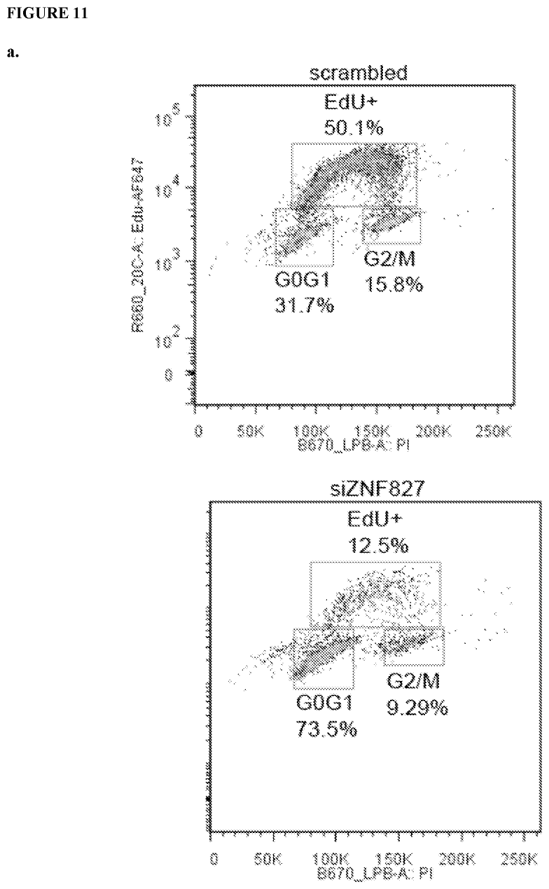

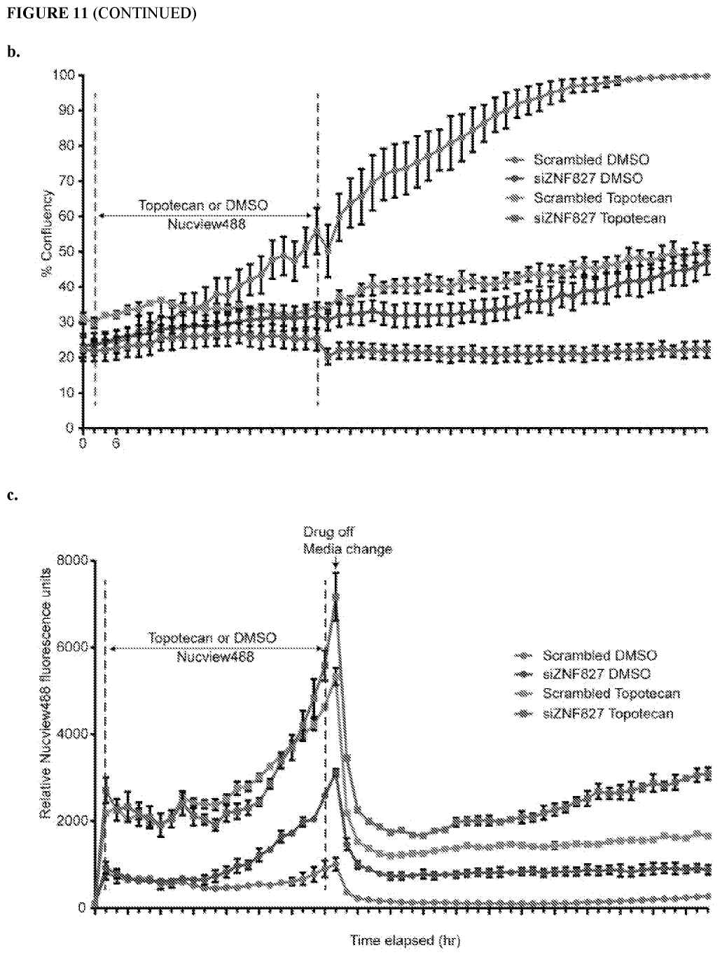

Depletion of ZNF827 caused a conspicuous G1 arrest and a significant reduction of S phase cells (FIG. 11a).

Considering the effects of ZNF827 depletion on DNA replication and the cell cycle, the impact of ZNF827 on cell proliferation and cell viability was examined. Live cell assays were performed on U-2 OS cells in the IncuCyte® to monitor real-time cell proliferation by confluency and cell viability using Nucview488, a fluorescent reporting dye for caspase 3 activity that allows quantitative measurement of apoptotic activity. Cells were treated with topotecan or DMSO control for the first half of the assay and treatments were removed for the remaining time.

Cells were seeded at 20-30% in a 96-well black plate with clear bottom (3603, Corning) and left to adhere at 37° C. with 10% CO2 for at least 3 hours. The media was aspirated and replaced with phenol red free media supplemented 2 μM Nucview® 488 Caspase-3 substrate (Biotium) and placed into the IncuCyte® for baseline imaging without drug treatment. Topotecan 2 μg/mL or DMSO control was added to the wells, and incubated at 37° C. with 10% CO₂ for 1 hour. The media was then aspirated, cells were washed carefully once with warm PBS, and the media replaced with phenol red free media supplemented 2 μM Nucview® 488 Caspase-3 substrate (Biotium). The plate was then placed back into IncuCyte®, imaged immediately post drug treatment, and then every 4 hours for 72 to 84 hours in the phase and green channels. Image analysis was performed with the IncuCyte® Zoom software. Cell growth was measured as % confluency over time, and apoptotic activity as green object confluence/area over time.

Topotecan effectively inhibited cell growth and its inhibitory effects lasted until the end of the experiment, even after drug removal (FIG. 11b). Consistent with G1 and early S phase arrest, ZNF827 depletion reduced cell proliferation, surprisingly to a similar efficacy as topotecan. ZNF827 depletion in conjunction with topotecan completely abrogated cell proliferation, indicative of a synergistic effect (FIG. 11b). Furthermore, ZNF827 depletion alone caused significantly higher apoptotic activity compared to scrambled control (FIG. 11c). Topotecan triggered much greater apoptotic activity as expected, which was exceeded markedly by the combination of ZNF287 depletion and topotecan towards the end of the drug-on period until the end of the experiment (FIG. 11c). This suggests that ZNF827 inhibition confers sensitisation to topotecan treatment, perhaps by muting ATR-CHK1 activation that promotes DNA repair and cell survival. The antiproliferative and apoptotic effects of ZNF827 depletion coincided with p21 induction, suggesting p21 dependent growth arrest and apoptosis. Together, these data suggest that ZNF827 loss is detrimental to cell proliferation and sensitises cancer cells to anti-cancer agents such as topotecan. These findings can be exploited for synthetic lethality.

### Example 14. Decreased ZNF827 Expression Levels Increase Drug-Sensitivity to Anti-Cancer Agents

Gene expression data (CCLE_expression.csv; DepMap Public 19Q2, Cancer Cell Line Encyclopedia Consortium, and Genomics of Drug Sensitivity in Cancer Consortium, 2015; Barretina et al., 2012) and drug sensitivity data (Sanger_GDSC_IC50.csv, Sanger GDSC v17.3, Iorio et al., 2016) were downloaded from depmap (https://depmap.org/portal/download/). Both datasets were intersected and filtered in order to identify 660 cell lines with ZNF827 gene expression data that contained drug sensitivity data from 266 compounds. ZNF827 gene expression for the 660 cell lines was plotted on a histogram and grouped by gene expression level, represented as log₂(TPM), into “low” (<1), “normal” (>1, <5) and “high” (>5). As only 2 cell lines were identified as “high” ZNF827 expression so were not included in statistical analysis. The Nemenyi statistical test was used to test for significant differences between “low” and “normal” ZNF827 expression groups for drug sensitivity for each compound. P-values were then corrected for multiple testing using FDR correction. The non-parametric Nemenyi test was chosen as tests for equal variance between the groups (Levene's test) and normality (Shapiro-Wilk test) showed significant differences in variance and significant departure from normality, respectively. All statistical analysis was performed in R (version 3.6.0), using the “stats” (version 3.6.0) and “PMCMR” (version 4.3) libraries.

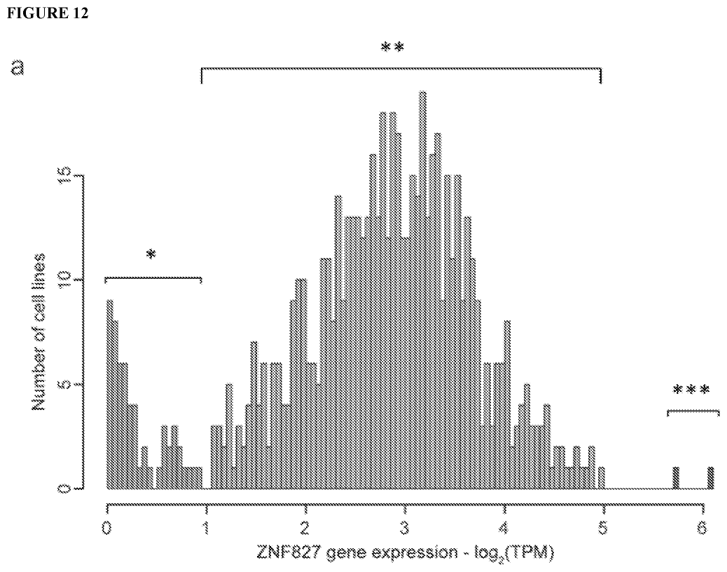

Drug-sensitivity comparisons between cell lines expressing low levels of ZNF827 and “normal” expression levels of ZNF827 (FIG. 12). The data was sourced from Sanger GDSC v17.3 (Iorio et al., 2016). Increased sensitivity was demonstrated in cell lines with low ZNF827 compared to cell lines with normal ZNF expression for AKT inhibitor VIII (CAS no. 612847-09-3), Axitinib (CAS no. 319460-85-0), AZ628 (CAS no. 878739-06-1), Bexarotene (CAS no.153559-49-0), CI-1040 (CAS no. 212631-79-3), FMK (CAS no. 821794-92-7), FR-180204 (CAS no. 865362-74-9), GW441756 (PubChem CID 16219401), I-BET-762 (CAS no. 1260907-17-2), Imatinib (CAS no. 152459-95-5), KIN001-236, KIN001-244 (CAS no. 1001409-50-2), KIN001-260 (PubChem CID 10451420), Nilotinib (CAS no. 641571-10-0), NPK76-II-72-1 (PubChem CID 46843648), NVP-BHG712 (CAS no. 940310-85-0), OSI-930 (CAS no. 728033-96-3), PD0325901 (CAS no. 391210-10-9), Phenformin (CAS no. 114-86-3), SNX-2112 (CAS no. 908112-43-6), Sunitib (CAS no. 341031-54-7), T0901317 (CAS no. 293754-55-9), TAK-715 (CAS no. 303162-79-0), Tamoxifen (CAS no. 10540-29-1), THZ-2-102-1, THZ-2-49, TL-1-85, TL-2-105, Tretinoin (CAS no. 302-79-4), VNLG-124, Vorinostat (CAS no. 149647-78-9) and (CAS no. VX-702 745833-23-2).

### Example 15. ZNF827 Depletion Increases Drug-Sensitivity to Anti-Cancer Agents

U-2 OS cells were plated in a 96-well plate at 4000 cells per well. 24 hours following plating, cells were transfected with either scrambled or ZNF827 siRNAs (Table 2) using Lipofectamine RNAiMAX transfection reagent (ThermoFisher Scientific). 24 hours post siRNA transfection, cells were treated with topotecan at a range of concentrations including 0.0039, 0.0078, 0.0156, 0.03125, 0.0625, 0.125, 0.25, 0.5 and 1 μg/mL in triplicates. DMSO was used as untreated control. Immediately after treatment addition, cells were imaged using the IncuCyte® live cell imaging system at 2-hour intervals for 180 hours. Cell proliferation was measured by phase object confluence calculated by the IncuCyte® analysis software. For IC50s, cell proliferation data were analysed in GraphPad Prism 8 to obtain area under curve (AUC), followed by normalisation and non-linear regression using the log(inhibitor) vs. normalised response -variable slope' function.

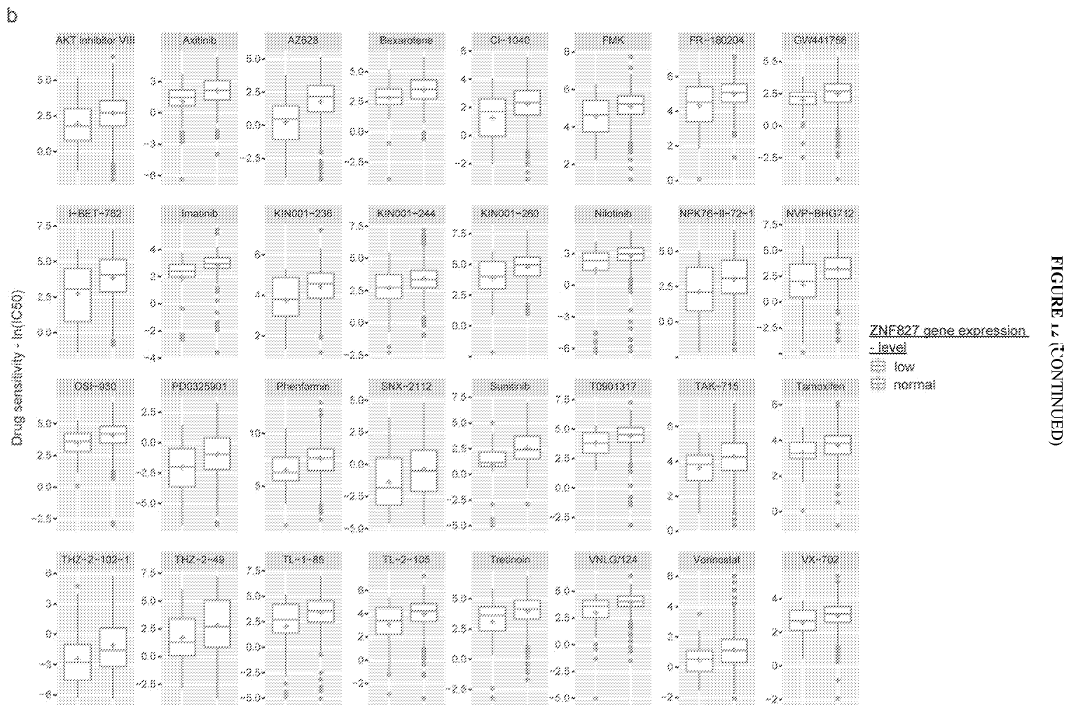

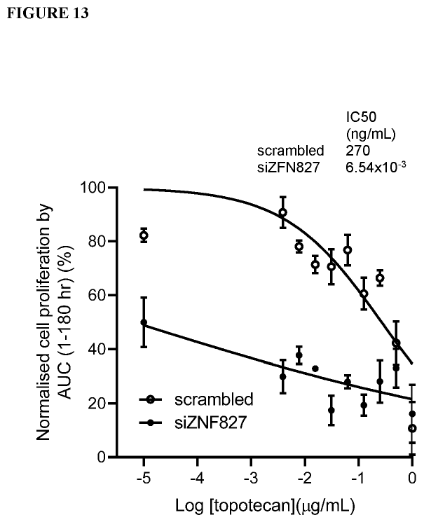

As shown in FIG. 13, cells transfected with ZNF827 siRNAs show markedly increased sensitivity to topotecan treatment compared to cells transfected with the scrambled control.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### REFERENCES

Barretina J et al., Nature. 483:603-7 (2012)
Cancer Cell Line Encyclopedia Consortium, and Genomics of Drug Sensitivity in Cancer Consortium. Nature. 528:84-87 (2015).
Cimprich K A and Cortez D Nat. Rev. Mol. Cell Biol. 9, 616-627 (2008).
Cohen S B and Reddel R R, Nat. Methods. 5, 355-360.
Conomos D et al., Nat. Struct. Mol. Biol. 21, 760-770 (2014).
Dejardin J and Kingston R E Cell. 136, 175-186 (2009).
Dunham M A, et al., Nat. Genet. 26, 447-450 (2000).
Essers, J, et al., Mol. Cell. Biol. 25, 9350-9359 (2005).
Flynn R L and Zou L Trends Biochem Sci. 36, 133-40 (2011).
Flynn R L, et al., Science. 347, 273-277 (2015).
Gao X D, et al., Nucleic Acids Res. 31, e124 (2003).
Garcia-Exposito L et al., Cell Reports. 17, 1858-1871 (2016).
Gartel A L, and Radhakrishnan S K Cancer Res. 65, 3980-5 (2005).
Heaphy C M et al., Am J Pathol. 179(4):1608-1615 (2011).
Henson J D, et al., Nat. Biotechnol. 27, 1181-1185 (2009).
Jiang, W Q, et al., Mol. Cell. Biol. 25, 2708-2721 (2005).
Lau M S et al., Nucleic Acids Res. 41, e34 (2013).
Lee M et al., Nucleic Acids Res. 146(10):4903-4918. (2018).
Murnane J P, et al., EMBO J. 13, 4953-4962 (1994).
Niculescu A B R et al., Mol. Cell. Biol. 18, 629-643 (1998).
Ogryzko V V et al., Mol. Cell. Biol. 17, 4877-4882 (1997)
Perrem, K, et al., Mol. Cell. Biol. 21, 3862-3875 (2001).
Radhakrishnan S K et al., Oncogene. 23, 4173-4176 (2004).
Sorensen C S et al., Nat. Cell Biol. 7, 195-201 (2005).
Syljuasen R G et al., Radiother Oncol. 75, 237-45 (2005)
Voter A F, et al., J. Biomol. Screen. 21, 626-633 (2016).
Waga S et al., Nature. 369, 574-578 (1994).
Yeager, T R, et al., Cancer Res. 59, 4175-4179 (1999).
Zou Y et al., J. Cell. Physiol. 208, 267-273 (2006).

1. A method of treating cancer, comprising inhibiting ZNF827 in a subject in need thereof, wherein the subject is receiving simultaneous, separate or sequential treatment with an anti-cancer agent.

2. A method of sensitizing a cancer cell to an anti-cancer agent, comprising inhibiting ZNF827.

3. The method according to claim 1 or claim 2, wherein the anti-cancer agent is a DNA-damaging agent.

4. The method according to any one of claims 1-3, wherein the anti-cancer agent is an alkylating agent, an antimetabolite, an anti-tumour antibiotic, a topoisomerase inhibitor, a mitotic inhibitor, a corticosteroid or a PARP inhibitor.

5. The method according to any one of claims 1-4, wherein the anti-cancer agent is selected from the group consisting of: irinotecan, camptothecin, etoposide, teniposide, doxorubicin, olaparib, rucaparib, niraparib, AKT inhibitor VIII, Axitinib, AZ628, Bexarotene, CI-1040, FMK, FR-180204, GW441756, I-BET-762, Imatinib, KIN001-236, KIN001-244, KIN001-260, Nilotinib, NPK76-II-72-1, NVP-BHG712, OSI-930, PD0325901, Phenformin, SNX-2112, Sunitib, T0901317, TAK-715, Tamoxifen, THZ-2-102-1, THZ-2-49, TL-1-85, TL-2-105, Tretinoin, VNLG-124, Vorinostat and VX-702 and topotecan.

6. The method according to any one of claims 1-5, wherein the anti-cancer agent is topotecan.

7. A method of treating cancer, comprising inhibiting ZNF827 in a subject in need thereof, wherein the cancer is not an ALT cancer.

8. The method according to any one of claims 1-7, wherein inhibiting ZNF827 comprises administering an inhibitor of ZNF827.

9. The method according to claim 8, wherein the inhibitor is any one or more of a genetic inhibitor, a small molecule, a peptide and a protein.

10. The method according to claim 9, wherein the genetic inhibitor is siRNA.

11. The method according to any one of claims 1-10, wherein inhibiting ZNF827 comprises disrupting the binding of ZNF827 to an endogenous binding partner at the N-terminal RRK motif of ZNF827.

12. The method according to any one of claims 1-10, wherein inhibiting ZNF827 comprises disrupting the binding of ZNF827 to an endogenous binding partner at any one or more zinc finger domain of ZNF827.

13. The method according to any one of claims 1-10, wherein inhibiting ZNF827 comprises disrupting the binding of ZNF827 to an endogenous binding partner at any one or more sumoylation site of ZNF827.

14. A method of selecting a subject for treatment with an inhibitor of ZNF827, the method comprising determining the level of expression and/or activity of ZNF827 in the subject, wherein if the level of expression and/or activity of ZNF827 in the subject is normal, the subject is selected for treatment with the inhibitor of ZNF827.

15. A method of selecting a subject for treatment with an anti-cancer agent, the method comprising determining the level of expression and/or activity of ZNF827 in the subject, wherein if the level of expression and/or activity of ZNF827 in the subject is low, the subject is selected for treatment with the anti-cancer agent.

16. A method of predicting the response of a subject to an anti-cancer agent, the method comprising determining the level of expression and/or activity of ZNF827 in the subject, wherein a low level of expression and/or activity of ZNF827 in the subject is indicative that the subject's response to the anti-cancer agent is likely improved relative to if the subject had a normal level of expression of ZNF827.

17. A method of identifying whether a subject suffering from cancer is suitable for treatment with an inhibitor of ZNF827, the method comprising determining the level of expression and/or activity of ZNF827 in the subject, wherein if the level of expression and/or activity of ZNF827 in the subject is normal, the subject is identified as being suitable for treatment with the inhibitor of ZNF827.

18. A method of identifying whether a subject suffering from cancer is suitable for treatment with an anti-cancer agent, the method comprising determining the level of expression and/or activity of ZNF827 in the subject, wherein if the level of expression and/or activity of ZNF827 in the subject is low, the subject is identified as being suitable for treatment with the anti-cancer agent.

19. A pharmaceutical composition comprising an inhibitor of ZNF827 and an anti-cancer agent.

20. A pharmaceutical composition comprising an inhibitor of ZNF827 and an anti-cancer agent for use in treating cancer.

21. A pharmaceutical composition comprising an inhibitor of ZNF827 for use in treating cancer, wherein the cancer is not an ALT cancer.

22. A method of preparing the pharmaceutical composition according to claim 20, comprising combining an inhibitor of ZNF827 and an anti-cancer agent.

23. Use of an inhibitor of ZNF827 and an anti-cancer agent in the manufacture of a medicament for the treatment of cancer.

24. Use of an inhibitor of ZNF827 in the manufacture of a medicament for the treatment of cancer, wherein the cancer is not an ALT cancer.

25. The pharmaceutical composition according to claim 21, wherein the pharmaceutical composition consists essentially of an inhibitor of ZNF827; or the use according to claim 24, wherein the medicament consists essentially of an inhibitor of ZNF827.

26. The pharmaceutical composition according to claim 19, or the use according to claim 23, or the method according to claim 22, wherein the anti-cancer agent is a DNA-damaging agent.

27. The pharmaceutical composition according to claim 19 or claim 26, or the use according to claim 23 or claim 26, or the method according to claim 22 or claim 26, wherein the anti-cancer agent is an alkylating agent, an antimetabolite, an anti-tumour antibiotic, a topoisomerase inhibitor, a mitotic inhibitor, a corticosteroid or a PARP inhibitor.

28. The pharmaceutical composition according to any one of claim 19, 26 or 27, or the use according any one of claim 23, 26 or 27, or the method according to any one of claim 22, 26 or 27, wherein the anti-cancer agent is selected from the group consisting of: irinotecan, camptothecin, etoposide, teniposide, doxorubicin, olaparib, rucaparib, niraparib, AKT inhibitor VIII, Axitinib, AZ628, Bexarotene, CI-1040, FMK, FR-180204, GW441756, I-BET-762, Imatinib, KIN001-236, KIN001-244, KIN001-260, Nilotinib, NPK76-II-72-1, NVP-BHG712, OSI-930, PD0325901, Phenformin, SNX-2112, Sunitib, T0901317, TAK-715, Tamoxifen, THZ-2-102-1, THZ-2-49, TL-1-85, TL-2-105, Tretinoin, VNLG-124, Vorinostat and VX-702 and topotecan.

29. The pharmaceutical composition according to any one of claim 19-21 or 25-28, or the use according to any one of claims 23-28, or the method according to any one of claim 22 or 26-28, wherein the inhibitor of ZNF827 is any one or more of a genetic inhibitor, a small molecule, a peptide and a protein.
